(19) United States
(12) Patent Application Publication
(10) Pub. No.: US 2023/0212634 A1
(43) Pub. Date: Jul. 6, 2023

(54) Expression of Ovalbumin and its Natural Variants

(71) Applicant: The Protein Brewery B.V., Breda (NL)

(72) Inventors: Joyce Josephine Elisabeth Arnouts, Achtmaal (NL); María Vázquez Vitali, Gent (BE); Lucie Parenicová, The Hague (NL); Niels Lauret, Prinsenbeek (NL); Maloe Kleine Haar, Nijmegen (NL); Jos Reijngoud, Alphen aan den Rijn (NL); Stefan Robert Marsden, The Hague (NL); Wilhelmus Theodorus Antonius Maria De Laat, Breda (NL)

(73) Assignee: The Protein Brewery B.V., Breda (NL)

(21) Appl. No.: 17/791,940

(22) PCT Filed: Jan. 14, 2021
(86) PCT No.: PCT/EP2021/050650

§ 371 Date: Jul. 11, 2022

(30) Foreign Application Priority Data
Jan. 14, 2020 (EP) 20151815.6

(51) Int. Cl. C12P 21/02; C12N 9/34

(52) U.S. Cl. CPC C12P 21/02; C12N 9/2428; C12Y 302/01003

(57) ABSTRACT

The present invention relates to the animal-free production of animal-derived proteins for human consumption by expression of such proteins, e.g. ovalbumin, in fungal cells. The invention relates to fungal cells modified for the production of animal-derived proteins, such as ovalbumin and to methods wherein these cells are used for the production of animal-derived proteins.

### FIELD OF THE INVENTION

The present invention relates to the field of molecular microbiology, food technology and fermentation technology. In particular, the invention relates to the animal-free production of animal-derived proteins for human consumption by expression of such proteins, e.g. ovalbumin in fungal cells. The invention relates to fungal cells modified for the production of animal-derived proteins, such as ovalbumin and to methods wherein they are used to produce such proteins.

### BACKGROUND OF THE INVENTION

Egg white is used in many food applications for binding, foaming and gelling purposes. With the global population anticipated to rise from 7 billion towards 9 billion in 2050 and 11 billion in 2100, global food production needs to grow drastically. Concurrent to this, due to global warming, there is a reduction in available arable land surface and sweet irrigation water. The process of egg production requires significant amounts of water, via the cultivation of soybean and grains, which are fed to chickens. Additional considerations of this process are the outputs which include nitrogen emissions via manure, the threat to human health by Salmonella and Listeria in egg products, and the increase of antibiotic resistance caused by the use of antibiotics in chicken farms. The health of humans is further threatened by the use of large amounts of pesticides like Fipronil, which is used to fight mites in chicken farms and may accumulate in the eggs themselves. With these concerns in mind, there is an increased demand for plant based food products with good nutritional value and functional properties, especially from vegan consumers (eating no animal derived products).

The concept of expressing egg white proteins like ovalbumin in microbes, and cultivating them in sterile fermenters can be considered as an alternative for obtaining functional ingredients like egg white. In order to produce egg white proteins by fermentation, preferably the production should be more land- and water-efficient, with less contribution to global warming factors. The production of ovalbumin per hectare was calculated by assuming an average of 7 tonnes of chicken feed per hectare (10 tonnes of corn and 4 tonnes of soybean per HA), then assuming 2 kg of chicken feed per kg egg and 6% egg white protein of the egg (the rest is shell, egg yolk and water). Assuming 54% of ovalbumin in egg white protein, the total production of ovalbumin by the animal value chain system is 7 * 0.06 * 0.54/2 = 113 kg of ovalbumin obtained per year.

The production of proteins from sugar, using ammonia as a nitrogen source in a fungal system can be as high as 20% on weight basis. The proteins produced by fungi can be secreted from the cell into the culture medium and then the proteins of interest can be harvest from the fermenter by separating the cells from the secreted proteins by microfiltration, filtration, centrifugation or a combination thereof. The cell free protein solution may then be concentrated by ultrafiltration or vacuum evaporation if necessary, towards the desired protein concentration. Subsequently, the protein may be further purified from fungal background proteins that may be present, such as cellulases (Trichoderma) or amylases (Aspergillus). The proteins can then be dried to powders by methods known in the art, such as freeze drying or spray drying.

An alternative method may use water efficient, high yielding sugar crops, such as sugar cane or sugar beet, or corn. Therefore, more than 15-20 tonnes of sugar can be harvested per hectare per year. Assuming a 20% sugar yield and 17.5 tonnes of sugar per hectare, 3500 kg of ovalbumin can theoretically be harvested per HA. This is 30 times more than the current egg production process described earlier.

As fungi can produce up to 100 g/L protein and yeasts only 10 g/L at the same sugar input, there it may be considered that using yeasts will provide only 3 times improvement of the animal system, while with fungi we can reach 30 times higher land use reduction. Many fungi and yeasts have been exploited on an industrial scale for production of enzymes and other proteins. The most utilized yeasts are Saccharomyces cerevisiae, Pichia pastori, Kluyveromyces lactis and Hansenula polymorpha. While for K. lactis the highest reported protein expression is around few g/L of fermentation broth (van Ooyen et al., 2006), for P. pastoris it is about a factor two higher (Werten M.W.T. et al., 2019). However, a production process, that will be based on working with methylotrophic yeasts (P. pastoris), requires installation of explosion-proof fermentation equipment due to the use of promoters, which rely on methanol as the inducer. Most industrial enzymes are produced by filamentous fungi such as Aspergillus spp., Trichoderma reesei, Myceliophthora thermophila etc. Filamentous fungi are known for their extremely high protein secretion capacity, which is several times higher than that of yeasts. For instance, for M. thermophila, previously known as Chrysosporium lucknowense C1, which was developed by Dyadic International Inc. (US) for production of industrial and pharma proteins and enzymes, the production titres of secreted proteins higher than 100 g/L have been reported (Visser H. et al., 2011). For A. niger and T. reesei the production titres are believed to be in the range of several dozens of g/L, possibly reaching more than 100 g/L of protein (Owen ward, 2012).

Hen ovalbumin has been expressed in yeast S. cerevisiae (Mercereau-Puijalon O. et al., 1980) and P. pastoris (Ito K. et al., 2005). The total secreted amount of ovalbumin reported by Ito et al. was about 10 mg/L, while ovalbumin was found in the cell-free extract in the amount between 5-20 ng/mg of protein in S. cerevisiae. The P. pastoris secreted ovalbumin was found to be mono-or di-glycosylated and no N-terminal acetylation and no phosphorylation were found when compared to hen ovalbumin (Ito K. et al., 2005, supra). The hen ovalbumin molecule is mainly mono-glycosylated, N-terminal acetylated and phosphorylated at zero, one or two serine residues (Nisbet A. D. et al., 1981). Clara Foods CO., San Francisco, CA (US) describes in their patent application (US 2018355020 A1) cloning and overexpression of several proteins present in a hen egg in P. pastoris, including ovalbumin. The amount of ovalbumin produced is however not mentioned.

There are reports in the literature of heterologous expression of ovalbumin originating from different birds. Křížková et al. (1992) isolated cDNA from Japanese quail and expressed it in S. cerevisiae. The protein was detected in cell-free extract. Yang et al. (2009) described the cloning of a Chinese quail ovalbumin gene and overexpression in P. pastoris, with reported yields up to 5.45 g/L of secreted ovalbumin.

There is however, still a need in the art for more efficient microbial production of animal-derived proteins for human consumption, such as meat, dairy and poultry proteins, including egg white proteins such as ovalbumin. The present invention provides means and methods for the expression of such animal-derived proteins in filamentous fungi.

### SUMMARY OF THE INVENTION

In one aspect, the invention relates to a fungal host cell comprising an expression cassette comprising a nucleotide sequence coding for an animal-derived food-protein of interest, which nucleotide sequence is operably linked to at least one regulatory sequence that is capable of effecting expression of the encoded protein of interest by the fungal host cell.

In one embodiment, there is provided, the fungal host cell according to the invention, wherein the regulatory sequence is the promoter of a highly expressed fungal protein, wherein preferably the highly expressed fungal protein is selected from acid α-amylase, α-amylase, TAKA-amylase, glucoamylase, xylanase, cellobiohydrolase, pyruvate kinase, glyceraldehyde-phosphate dehydrogenase, alcohol dehydrogenase, aldehyde dehydrogenase, sucrase, acetamidase, superoxide dismutase, more preferably wherein the promoter is an A. niger glucoamylase promoter.

In one embodiment, there is provided, the fungal host cell according to the invention, wherein the nucleotide sequence coding for the animal-derived food-protein of interest is codon optimized with reference to the native codon usage of the highly expressed fungal protein from which the promoter is derived, wherein preferably the highly expressed fungal protein is an A. niger glucoamylase.

In one embodiment, there is provided, the fungal host cell according to the invention, wherein the expression cassette is integrated in a locus of a gene coding for a highly expressed fungal protein and wherein preferably, the locus is an amplified locus, wherein more preferably, the locus is an A. niger glaA locus. In some embodiment, the expression cassette is integrated in the locus by gene replacement, wherein preferably, the expression cassette replaces more than one copy of the gene coding for the highly expressed fungal protein in the fungal genome, wherein more preferably, the locus is the an A. niger glaA locus.

In one embodiment, there is provided, the fungal host cell according to the invention, wherein the expression cassette comprises a nucleotide sequence encoding a signal sequence from a highly expressed secreted fungal protein, and optionally a pro-sequence, operably linked in frame to the nucleotide sequence coding forthe animal-derived food-protein of interest, wherein preferably the signal sequence and optional a pro-sequence are the A. niger glucoamylase prepro-sequences.

In one embodiment, there is provided, the fungal host cell according to the invention, wherein the expression cassette encodes a fusion protein comprising in a N- to C-terminal direction: an A. niger glucoamylase pre-pro sequence; optionally, 10, 20, 30, 40, 50, 60, 70, 80, 90 or 100% of the mature A. niger glucoamylase amino acid sequence; optionally, a cleavable linker polypeptide; fused to the N-terminus of the protein of interest.

In one embodiment, there is provided, the fungal host cell according to the invention, wherein the host cell is a yeast or a filamentous fungus, preferably, a filamentous fungal host cell belong to a species selected from Alternaria altemata, Apophysomyces variabilis, Aspergillus spp., Aspergillus fumigatus, Aspergillus flavus, Aspergillus oryzae, Aspergillus niger, Aspergillus awamori, Aspergillus sojae, Aspergillus nidulans, Aspergillus terreus, Cladosphialophora spp., Fonsecaea pedrosoi, Fusarium spp., Fusarium oxysporum, Fusarium solani, Lichtheimia spp., Lichtheimia corymbifera, Lichtheimia ramosa, Myceliophthora spp., Myceliophthora thermophila, Rhizopus spp., Rhizopus microsporus, Rhizomucor spp., Rhizomucor pusillus, Rhizomucor miehei, Trichoderma spp., Trichoderma reesei Trhichophyton spp., Trichophyton interdigitale, and Trichophyton rubrum, of which a strain of the species Aspergillus niger is most preferred.

In one embodiment, there is provided, the fungal host cell according to the invention, wherein the host cell is a strain of a filamentous fungus that has the ability to grow in a yeast-like morphology, wherein preferably the strain is Aspergillus niger strain CICC2462, or a strain that is a single colony isolate and/or a derivative of strain CICC2462.

In one embodiment, there is provided, the fungal host cell according to the invention, wherein animal-derived food-protein of interest is a hemeprotein, a milk protein or an egg protein, preferably an egg white protein and most preferably an ovalbumin.

In one embodiment, there is provided, the fungal host cell according to the invention, wherein the ovalbumin comprises an amino acid sequence with at least 50% identity to the amino acid sequence of an ovalbumin from a bird selected from the group consisting of chicken, pelican, quail, pigeon, ostrich, plover, turkey, duck, goose, gull, guinea fowl, jungle fowl, peafowl, partridge, pheasant, emu, rhea and kiwi.

In a further aspect, there is provided a process for producing an animal-derived food-protein of interest, the process comprising the steps of:
a) culturing the fungal host cell as defined in any one of claims 1 to 10 in a medium in a fermenter under condition conducive to the expression of the protein of interest; and,
b) optionally, recovery of the protein of interest, wherein, preferably, the protein of interest is an ovalbumin.

In one embodiment, there is provided, the process according to the invention, wherein in step a) the fungal host cell is cultured at a pH that is equal to or higher than pH 5.0, 5.5, 6.0, 6.5, 7.0, 7.5, 8.0 or 8.5 and/or that is equal to or lower than 10, 9.5. 9.0, 8.5, 8.0, 7.5, or 7.0.

In one embodiment, there is provided, the process according to the invention, wherein in step a) the input of mechanical power into the medium in the fermenter is no more than 2.5, 2.0, 1.8, 1.6, 1.4, 1.0, 0.5, 0.2 or 0.1 kW/m³.

In one embodiment, there is provided, the process according to the invention, wherein in step a) the fungal host cell is cultured in a fermenter equipped with a bubble column and wherein preferably the host cell is cultured without the input of mechanical power into the medium in the fermenter.

In a further aspect, there is provided the use of the fungal host cell according to the invention or the process according to the invention, for the production of an animal-derived food-protein of interest, wherein, preferably, the protein of interest is an ovalbumin.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

For purposes of the present invention, the following terms are defined below.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, ... ,etc.

The word “about” or “approximately” when used in association with a numerical value (e.g. about 10) preferably means that the value may be the given value (of 10) more or less 0.1% of the value.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty = 50 (polynucleotides) / 8 (proteins) and gap extension penalty = 3 (nucleotides) / 2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score = 100, wordlength = 12 to obtain nucleotide sequences homologous to the nucleic acid molecules of the invention. BLAST protein searches can be performed with the BLASTx program, score = 50, wordlength = 3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | GIn (Q) |
| Aliphatic Uncharged Residues | Gly (G), Ala (A), Val (V), Leu (L), and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes.

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative Physical and Functional Classifications of Amino Acid Residues.

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P and T |
| Flexible residues | Q, T, K, S, G, P, D, E, and R |

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in sodium chloride/sodium citrate (SSC) at about 45° C., followed by one or more washes in 1 X SSC, 0.1% SDS at about 50° C., preferably at about 55° C., preferably at about 60° C. and even more preferably at about 65° C.

Highly stringent conditions include, for example, hybridization at about 68° C. in 5x SSC/5x Denhardt’s solution / 1.0% SDS and washing in 0.2x SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or “expression construct” refer to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. An inducible promoter may also be present but not induced.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region, exons, introns and a 3′-nontranslated sequence (3′-end) e.g. comprising a polyadenylation- and/or transcription termination site.

“Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO’s) (self-cloning is defined herein as in European Directive 98/81/EC Annex ll). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed earlier herein.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which it is introduced, but have been obtained from another cell or synthetically or recombinantly produced. Generally, though not necessarily, such nucleic acids encode proteins, i.e. exogenous proteins, that are not normally produced by the cell in which the DNA is transcribed or expressed. Similarly exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other. The terms heterologous and exogenous specifically also apply to non-naturally occurring modified versions of otherwise endogenous nucleic acids or proteins.

The “specific activity” of an enzyme is herein understood to mean the amount of activity of a particular enzyme per amount of total host cell protein, usually expressed in units of enzyme activity per mg total host cell protein. In the context of the present invention, the specific activity of a particular enzyme may be increased or decreased as compared to the specific activity of that enzyme in an (otherwise identical) wild type host cell.

The term “fermentation” or “fermentation process” is herein broadly defined in accordance with its common definition as used in industry as any (large-scale) microbial process occurring in the presence or absence of oxygen, comprising the cultivation of at least one microorganism whereby preferably the microorganism produces a useful product at the expense of consuming one or more organic substrates. The term “fermentation” is herein thus has a much broader definition than the more strict scientific definition wherein it is defined as being limited a microbial process wherein the microorganism extracts energy from carbohydrates in the absence of oxygen. Likewise, the term “fermentation product” is herein broadly defined as any useful product produced in a (large-scale) microbial process occurring in the presence or absence of oxygen.

### DETAILED DESCRIPTION OF THE INVENTION

The present inventors have surprisingly found that it is possible to efficiently produce bird’s egg proteins such as ovalbumins having the desired functional properties by expressing the ovalbumins in specifically designed fungal host cells that are used in optimized fermentation and recovery process.

### Fungal Host Cell

In a first aspect the invention therefore pertains to the genetic modification of a fungal host cell so as to enable the host cell to produce a protein of interest. A fungal host cell of the invention thus preferably comprises an expression cassette comprising a nucleotide sequence coding for a protein of interest (e.g. an ovalbumin), which nucleotide sequence is operably linked to at least one regulatory sequence that is capable of effecting expression of the encoded protein of interest by the fungal host cell.

A fungal host is a host cell that belongs to the “fungi”, which are herein defined as eukaryotic microorganisms, which include all species of the subdivision Eumycotina (Alexopoulos, C. J., 1962, In: Introductory Mycology, John Wiley & Sons, Inc., New York). The term fungus thus includes both filamentous fungi and yeast. “Filamentous fungi” are herein defined as eukaryotic microorganisms that include all filamentous forms of the subdivision Eumycotina and Oomycota (as defined by Hawksworth et al., In, Ainsworth and Bisby’s Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. A preferred filamentous fungal host cell is a host cell the belongs to a genus selected from the genera including, but are not limited to, Alternaria, Apophysomyces, Aspergillus, Cladosphialophora, Fonsecaea, Fusarium, Lichtheimia, Myceliophthora, Rhizopus, Rhizomucor, Trichoderma and Trichophyton. More preferably, a filamentous fungal host cell belong to a species selected from Alternaria altemata, Apophysomyces variabilis, Aspergillus spp., Aspergillus fumigatus, Aspergillus flavus, Aspergillus oryzae, Aspergillus niger, Aspergillus awamori, Aspergillus nidulans, Aspergillus terreus, Cladosphialophora spp., Fonsecaea pedrosoi, Fusarium spp., Fusarium oxysporum, Fusarium solani, Lichtheimia spp., Lichtheimia corymbifera, Lichtheimia ramosa, Myceliophthora spp., Myceliophthora thermophila, Rhizopus spp., Rhizopus microsporus, Rhizomucor spp., Rhizomucor pusillus, Rhizomucor miehei, Trichoderma spp., Trichoderma reesei Trhichophyton spp., Trichophyton interdigitale, and Trichophyton rubrum. The most preferred filamentous fungal host cell is a strain of the species Aspergillus niger.

Several strains of filamentous fungi are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), Chinese Centrum for Industrial Culture Collection (CICC) and Agricultural Research Service Patent Culture Collection, Northern Regional Research Center (NRRL), including e.g. the strains Aspergillus niger CBS 513.88, CBS124.903, and CICC2462; Aspergillus oryzae ATCC 20423, IFO 4177, ATCC 1011, CBS205.89, ATCC 9576, ATCC14488-14491, ATCC 11601 and ATCC12892; P. chrysogenum CBS 455.95, Penicillium citrinum ATCC 38065, Penicillium chrysogenum P2, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906 and Chrysosporium lucknowense ATCC44006 and derivatives thereof.

“Yeasts” are herein defined as eukaryotic microorganisms and include all species of the subdivision Eumycotina that predominantly grow in unicellular form. Yeasts may either grow by budding of a unicellular thallus or may grow by fission of the organism. Alternatively, a fungal host cell is a yeast host cell the belongs to a genus selected from the genera including Candida, Hansenula, Kluyveromyces, Pichia, Saccharomyces, Schizosaccharomyces, or Yarrowia, more preferably a species selected from the species Kluyveromyces lactis, Saccharomyces cerevisiae, Hansenula polymorpha, Yarrowia lipolytica and Pichia pastoris.

The term “fungal”, when referring to a protein or nucleic acid molecule thus means a protein or nucleic acid whose amino acid or nucleotide sequence, respectively, naturally occurs in a fungus.

In one particularly preferred embodiment, the filamentous fungal host cell is of a strain that grows, or has the ability to grow with a “yeast like morphology”. The term “yeast like morphology” as used herein for a filamentous fungal host cell, indicates that the filamentous fungus grows with short hyphae and little of branching of the hyphae. A reference strain of Aspergillus niger that grows with a yeast like morphology is strain CICC2462, as obtainable from the China Center of Industrial Culture Collection (CICC, Building 6, No. 24 Yard, Jiuxianqiao Middle Road, Chaoyang District, Beijing, China; www.china-cicc.org). Aspergillus niger CICC2462 is used in the industrial production of glucoamylase and is a morphological mutant strain of A. niger that does not produce spores, has short mycelia, thick hyphae, which results in a low-viscosity fermentation broth, is a strong enzyme producer, has low protease activity, is osmotolerant, and is suitable for high-density submerged liquid fermentation (Zhang et al., Microb Cell Fact. 2016; 15: 68).

A filamentous fungal strain that grows with a yeast like morphology is herein defined as a filamentous fungal strain with at least one of the characteristics of: a) the hyphae of the filamentous fungal strain are on average not more than 5, 10, 20, 50 or 100% longer than the hyphae of the reference strain CICC2462; and b) the hyphae of the filamentous fungal strain show on average not more than 5, 10, 20, 50 or 100% branching than the hyphae of the reference strain CICC2462, whereby preferably the filamentous fungal strain and the reference strain CICC2462 are grown under identical conditions. A preferred filamentous fungal host cell is Aspergillus niger strain CICC2462, or a strain that is a single colony isolate and/or a derivative of strain CICC2462.

The advantage of using as host cell a filamentous fungal strain that grows with a “yeast like morphology” is that the strain can be cultured under condition requiring a minimum input of mechanical power into the medium in the fermenter for aeration, which provides savings on costs for energy. In addition the use of less mechanical power reduces shear forces, thereby reducing destruction of proteins and the cells, which increases yield and facilitates filtration by reducing clogging of filters by cellular debris etc.

In one embodiment, a fungal host cell of the invention comprises a genetic modification that reduces or eliminates the specific activity or amount of at least one enzyme or protein selected from a glucoamylase (e.g. glaA), an amylase, such an acid stable alpha-amylase (e.g. amyA) or a neutral alpha-amylase (e.g. amyBI and amyBII), an oxalic acid hydrolase (e.g. oahA), a protein involved in mycotoxin biosynthesis and a protease, and a protein encoded by KU70, KU80, hdfA and hdfB or homologues thereof. Preferably in the host the genetic modification reduces the specific activity or amount of the enzyme or protein to no more than 90, 75, 50, 20, 10, 5, 2 or 1% of the specific activity or amount of the enzyme or protein compared to a corresponding host cell lacking the genetic modification, when cultivated under identical conditions. More preferably, the genetic modification completely eliminates the specific activity or amount of the enzyme or protein in the host cell.

The advantage of reducing or eliminating the expression in the host cell of one or more of glucoamylase (glaA), acid stable alpha-amylase (amyA) and neutral alpha- amylase (amyBl and amyBII) is not only that the cell’s energy and resources are not utilized for these byproducts and/or that downstream processing of the ovalbumin product of interest is simplified since there are fewer by-products present, but most importantly, in many of the food applications of ovalbumin the action of these enzymes on starch and starch-derived substrates is preferably avoided.

The advantage of reducing or eliminating the expression in the host cell of oxalic acid hydrolase (oahA) is that the host cell will produce less or no oxalic acid. Oxalic acid is an unwanted by-product in many applications such as food-applications. Furthermore, reducing or eliminating the production of oxalic by the host cell reduces the undesirable lowering of the pH of the culture medium by this acid. Avoiding such low pH requires less buffering, improves product yield and avoids aggregation/precipitation of the ovalbumin produced.

The advantage of reducing or eliminating the expression in the host cell of a protein involved in mycotoxin biosynthesis is that it reduces or avoids the formation of such toxins during the fermentation of product of interest, which is highly undesirable as these toxins present a health hazard to operators, customers and the environment.

The advantage of reducing or eliminating the expression in the host cell of a protease is reduces the negative impact of host cell proteases on the yield and quality of the ovalbumin product of interest. Preferred genetic modifications or reducing or eliminating the expression in the host cell of one or more proteases include e.g. prtT, which is a transcriptional activator of proteases in eukaryotic cells. Several fungal transcriptional activators of proteases have been recently described in WO 00/20596, WO 01/68864, WO 2006/040312 and WO 2007/062936. These transcriptional activators were isolated from A. niger, A. fumigatus, P. chrysogenum and A. oryzae. These transcriptional activators of protease genes can be used to improve a method for producing a polypeptide in a fungal cell, wherein the polypeptide is sensitive for protease degradation. When the host cell is deficient in prtT, the host cell will produce less proteases that are under transcriptional control of prtT. It is therefore advantageous when the host cell according to the invention is deficient in prtT. A host cell of the invention may further be deficient a major protease such as pepA.

For means and method for effecting a genetic modification that reducing or eliminating in the host cell the specific activity or amount of at least one enzyme or protein selected from a glucoamylase (e.g. glaA), an amylase, such an acid stable alpha-amylase (e.g. amyA) or a neutral alpha- amylase (e.g. amyBl and amyBII), an oxalic acid hydrolase (e.g. oahA), a protein involved in mycotoxin biosynthesis and a protease, reference is made to WO2011/009700, which is incorporated herein by reference.

A further genetic modification that can be applied in a host cell of the invention for reducing the background amount of (endogenous) secreted proteins is an inactivation or deletion of the amyR as described by Zhang et al. (2016, supra), which is incorporated herein by reference.

### Expression Cassette

In one aspect, there is provided an expression cassette for expression of a protein of interest in a fungal host cell according to the invention. Preferably, the expression cassette comprises a nucleotide sequence coding for an animal-derived food-protein of interest. Preferably the coding nucleotide sequence is operably linked to at least one regulatory sequence that is effects or controls expression of the encoded protein of interest by the fungal host cell. The expression regulatory sequence preferably at least includes a transcription regulatory sequence or promoter operably linked to the coding sequence. The expression cassette further preferably includes regulatory sequences such as translation initiation sequences, secretion signal sequences, transcription termination sequences, polyadenylation signals. Additional factors necessary or helpful in effecting expression may also be present, such as expression enhancer elements. In one embodiment the regulatory sequences are the regulatory sequences of a highly expressed fungal protein.

The promoter that is operably linked to the coding sequence in the expression cassette according to the invention can be a constitutive promoter, an inducible promoter or a hybrid promoter. Examples of preferred inducible promoters that can be used are a starch-, copper-, oleic acid- inducible promoters.

A preferred promoter is a promoter of a highly expressed fungal protein. Preferred promoters from highly expressed fungal genes include the promoters obtained from the genes encoding fungal or filamentous fungal acid α-amylase, α-amylase, TAKA-amylase, glucoamylase, xylanase, cellobiohydrolase, pyruvate kinase, glyceraldehyde-phosphate dehydrogenase, alcohol dehydrogenase, aldehyde dehydrogenase, sucrase, acetamidase and superoxide dismutase genes. Specific examples thereof include the promoters from genes encoding the A. oryzae TAKA amylase, the A. niger neutral alpha-amylase, the A. niger acid stable alpha-amylase, the A. niger or A. awamori glucoamylases (glaA). Particularly preferred promoters for use in filamentous fungal cells are the A. niger and A. awamori glucoamylase (glaA) promoters, or a functional part thereof. In a preferred embodiment, not only the promoter but also the further regulatory sequences such as translation initiation sequences, secretion signal sequences, transcription termination sequences, polyadenylation signals are from a highly expressed fungal protein as indicated, of which the A. niger and A. awamori glucoamylase (glaA) regulatory sequences are most preferred.

The expression cassette is preferably part of an expression vector, which in addition to the expression cassette can comprise additional sequence elements such as selectable marker genes, sequences for targeting integration at specific loci in the host cell’s genome and/or for autonomous replication.

The expression vector may be any vector (e.g., a plasmid or virus), which can be conveniently subjected to recombinant DNA procedures and can bring about the expression of the polynucleotide of interest. The choice of the vector will typically depend on the compatibility of the vector with host cell into which the vector is to be introduced. The vectors may be linear or closed circular plasmids. The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. An autonomously maintained cloning vector may comprise the AMA1 -sequence (see e.g. Aleksenko and Clutterbuck (1997), Fungal Genet. Biol. 21: 373-397). Alternatively, the vector may be one which, when introduced into the host cell, is integrated into the genome and replicated together with the chromosome(s) into which it has been integrated. Preferably, the integrative cloning vector comprises a DNA fragment, which is homologous to a DNA sequence in a predetermined target locus in the genome of the host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least 30 bp, 50 bp, 0.1 kb, 0.2 kb, 0.5 kb, 1 kb or 1.5 kb, preferably at least 2.0 kb, more preferably at least 2.5 kb or most preferably at least 3.0 kb.

Preferred homologous sequences for targeting the expression vector/cassette are sequences from highly expressed fungal genes such as sequences obtained from the genes encoding fungal or filamentous fungal acid α-amylase, α-amylase, TAKA-amylase, glucoamylase, xylanase, cellobiohydrolase, pyruvate kinase, glyceraldehyde-phosphate dehydrogenase, alcohol dehydrogenase, aldehyde dehydrogenase and sucrase. More preferably, the homologous sequences for targeting the expression vector/cassette are sequences that target to loci comprising highly expressed fungal genes, which loci are amplified in the fungal genome, such as the TAKA amylase genes in Aspergillus oryzae (e.g. in strain IF04177) or the amplified glaA locus of Aspergillus niger (see US6432672B1 and US8734782B2), e.g. in strains CBS 513.88 and CICC2462. In a preferred embodiment, the expression cassette is integrated in a locus of a gene coding for a highly expressed fungal protein. Preferably, said locus is a locus that is amplified in the fungal genome, wherein more preferably, the locus is an A. niger glaA locus.

In one embodiment, the expression cassette is integrated by (homologous) recombination via a single cross-over. Preferably, said locus is a locus that is amplified in the fungal genome, wherein more preferably, the locus is an A. niger glaA locus.

In another embodiment, the expression cassette is integrated by (homologous) gene replacement in a locus of a gene coding for a highly expressed fungal protein (i.e. double cross-over). Preferably, said locus is a locus that is amplified in the fungal genome and the expression cassette replaces several copies or each copy of the gene coding for the highly expressed fungal protein in the fungal host cell’s genome, wherein more preferably, the locus is an A. niger glaA locus.

In one embodiment, the fungal host cell comprises multiple copies of the expression cassette, preferably integrated into the genome of the fungal host cell. Preferably, the fungal host cell comprises at least 2, 3, 4, 5, 6, 7, 8, 9, 10, 12, 15, 20, 25 or 30 copies of the expression cassette, preferably integrated into the genome of the fungal host cell, more preferably at a predefined location, such as a locus comprising a highly expressed endogenous fungal gene.

The vectors preferably contain one or more selectable markers, which permit easy selection of transformed cells. Using the method of co-transformation, one vector may contain the selectable marker whereas another vector may contain the polynucleotide of interest or the nucleic acid construct of interest; the vectors are simultaneously used for transformation of the host cell. A selectable marker is a gene the product of which provides for biocide or viral resistance, resistance to heavy metals, prototrophy to auxotrophs, and the like. A selectable marker for use in a filamentous fungal host cell may be selected from the group including, but not limited to, amdS (acetamidase), argB (ornithine carbamoyltransferase), bar (phosphinothricinacetyltransferase), bleA (phleomycin binding), hygB (hygromycinphosphotransferase), niaD (nitrate reductase), pyrG (orotidine-5′-phosphate decarboxylase), sC (sulfate adenyltransferase), and trpC (anthranilate synthase), as well as equivalents from other species. Preferred for use in an Aspergillus host cell are the amdS (US5876988, US6548285B1) and pyrG genes of A. nidulans or A. oryzae and the bar gene of Streptomyces hygroscopicus. More preferably an amdS gene is used, even more preferably an amdS gene from A. nidulans or A. niger. A most preferred selection marker gene is the A. nidulans amdS coding sequence fused to the A. nidulans gpdA promoter (see EP 0635574 B1). AmdS genes from other filamentous fungi may also be used (US 6548285 B1).

Means and methods for constructing the expression vectors and cassettes of the present invention are well known to one skilled in the art (see, e.g., Sambrook and Russell, supra; and Ausubel et al., Current Protocols in Molecular Biology, Wiley InterScience, NY, 1995).

In one embodiment, the nucleotide sequence encoding the protein of interest in the expression cassette of the invention preferably is adapted to optimize its codon usage to that of the host cell in question. The adaptiveness of a nucleotide sequence encoding an enzyme to the general codon usage of a host cell may be expressed as codon adaptation index (CAI). The codon adaptation index is herein defined as a measurement of the relative adaptiveness of the codon usage of a gene towards the codon usage of highly expressed genes in a particular host cell or organism. The relative adaptiveness (w) of each codon is the ratio of the usage of each codon, to that of the most abundant codon forthe same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al, 2003, Nucleic Acids Res. 3J_(8):2242-51). An adapted nucleotide sequence preferably has a CAI of at least 0.2, 0.3, 0.4, 0.5, 0.6, 0.7, 0.8 or 0.9.

In a preferred embodiment the nucleotide sequence encoding the protein of interest in the expression cassette of the invention preferably is adapted to optimize its codon usage to that of a highly expressed protein in the host cell in question, preferably to the codon usage of a highly expressed protein that is endogenous to the host cell in question. More preferably, the nucleotide sequence encoding the protein of interest in the expression cassette of the invention is adapted to optimize its codon usage to that of a highly expressed fungal protein selected from acid α-amylase, TAKA-amylase, glucoamylase, xylanase, cellobiohydrolase, pyruvate kinase, glyceraldehyde-phosphate dehydrogenase, alcohol dehydrogenase, aldehyde dehydrogenase and sucrase. The nucleotide sequence encoding the protein of interest can be adapted to the codon usage of a highly expressed fungal protein e.g. by using an on-line available DNA optimizing tool such as OPTIMISER (http://qenomes.urv.es/OPTIM ISER/; Puigbo P. et al., 2007; Nucl Acids Res 35; W126-W131) or commercial service providers of codon optimized synthetic genes (e.g. GenScript, USA).

In a further preferred embodiment, the expression cassette comprises a nucleotide sequence encoding the protein of interest that operably linked to a promoter, wherein the coding sequence is codon optimized with reference to the coding sequence that is native to the promoter, whereby preferably, the promoter is a promoter that is native to a gene for a highly expressed fungal protein, whereby, more preferably the highly expressed fungal protein preferably is a highly expressed fungal protein as indicated above, of which glucoamylase is the most preferred.

In yet a further preferred embodiment, the expression cassette further comprises a DNA fragment, which is homologous to a DNA sequence in a predetermined target locus in the genome of the host cell for targeting the integration of the cloning vector to this predetermined locus, whereby preferably the target locus is the locus of the highly expressed fungal protein native to the promoter in the expression cassette and to which the codon usage of the nucleotide sequence encoding the protein of interest is optimized whereby, preferably the highly expressed fungal protein preferably is a highly expressed fungal protein as indicated above, of which A.niger glucoamylase is most preferred.

In one embodiment, the expression cassette comprises an N-terminal secretion signal sequence that is operably linked to the coding sequence of the protein of interest for directing secretion of the protein of interest from the fungal host cell. A “signal sequence” is an amino acid sequence which when operably linked to the amino-terminus of a protein of interest permits the secretion of such protein from the host fungus. Such signal sequences may be the signal sequence normally associated with the protein of interest (i.e., a native signal sequence) or may be derived from other sources (i.e., a signal sequence foreign or heterologous to the protein of interest). Signal sequences are operably linked to a heterologous polypeptide either by utilizing a native signal sequence or by joining a DNA sequence encoding a foreign signal sequence to a DNA sequence encoding the protein of interest in the proper reading frame to permit translation of the signal sequence and protein of interest. Preferred signal sequences for use in the present invention include signals derived from highly expressed secreted fungal proteins such as acid α-amylase, α-amylase, TAKA-amylase, glucoamylase, xylanase, cellobiohydrolase and sucrase, of which A.niger glucoamylase is most preferred.

In a further preferred embodiment, the expression cassette encodes the protein of interest as part of a fusion protein. Preferably, the expression cassette encodes a fusion protein wherein the protein of interest is fused at its N-terminus with at least a part of highly expressed secreted fungal protein. More preferably, the expression cassette encodes a fusion protein wherein the protein of interest is fused at its N-terminus with at least an N-terminal part of the highly expressed secreted fungal protein, which preferably includes at least the signal sequence of the highly expressed secreted fungal protein. The fusion protein can further also comprise the pro-sequence of the highly expressed secreted fungal protein and/or or further parts of the mature highly expressed secreted fungal protein. Alternatively, the signal sequence may contain the pre- prosequences. If the protein of interest is a secreted protein, the N-terminal part of the highly expressed secreted fungal protein can be fused to the N-terminus of the mature secreted protein of interest. Alternatively, the N-terminal may be fused to the start of the mature protein (e.g. afterthe processing to avoid possible misprocessing in the fungal cell). If the protein of interest is not a secreted protein, the N-terminal part of the highly expressed secreted fungal protein can replace the N-terminal methionine of the protein of interest.

It is further preferred that the expression cassette encodes a fusion protein wherein the protein of interest is fused to its N-terminal fusion partner through of cleavable linker polypeptide such as the prosequence of glucoamylase, the prosequence of bovine chymosin, the prosequence of subtilisin, prosequences of retroviral proteases including HIV protease and DNA sequences encoding amino acid sequences recognized and cleaved by trypsin, factor Xa, collagenase, clostripin, subtilisin, chymosin, yeast KEX2 protease and Aspergillus KEXB. Particularly preferred cleavable linkers are the KEX2 protease recognition site (Lys-Arg), which can be cleaved by a native Aspergillus KEX2-like (KEXB) protease.

In a preferred embodiment, the expression cassette encodes a fusion protein comprising in a N- to C-terminal direction: an A. niger glucoamylase pre-pro sequence; optionally, 10, 20, 30, 40, 50, 60, 70, 80, 90 or 100% of the mature A. niger glucoamylase amino acid sequence; optionally, a cleavable linker polypeptide and/or KEX2 (Lys-Arg) cleavage site; fused to the protein of interest. Examples thereof are described in the Examples herein: the first 502 amino acids of A. niger glucoamylase including the pre-pro sequence and a synthetic peptide of 8 amino acids which includes the KEX2 (Lys-Arg) cleavage site fused to a protein of interest, or further truncations of the A. niger glucoamylase with only 54 or 100 amino acids including the pre-pro sequences and before the KEX2 (Lys-Arg) cleavage site fused to the protein of interest.

### Animal-Derived Food-Proteins of Interest

The present invention concerns the animal-free production of proteins that are normally derived from animals and that are commonly used in the preparation of food for human consumption. In principle any protein that is normally obtained from or produced by an animal or part of an animal and that can be used in the preparation of food for human consumption is suitable for being produced in an animal-free manner in accordance with the invention. However, more particularly the invention is concerned with the animal-free production of dairy and poultry proteins, more specifically milk proteins and egg proteins.

In one embodiment, the animal-derived food-protein of interest is a milk protein, preferably a protein present in the milk of cattle (i.e. bovine or Bos taurus), buffalo (including water buffalo), goats, sheep or camel, or in the milk of other less common milk animals such as yak, horse, reindeer and donkeys. The protein of interest can be a casein or can be a whey protein such as β-lactoglobulin, α-lactalbumin, bovine serum albumin or an immunoglobulin.

In one embodiment, the animal-derived food-protein of interest is a hemeprotein, preferably a hemeprotein from a non-human animal, more preferably a mammal such as a cow, pig, horse, goat or sheep. Preferred animal-derived hemeproteins include hemoglobin and myoglobin. The animal-derived hemeproteins produced in accordance with the invention can be applied as red heme-bound iron protein in meat substitutes.

In one embodiment, the animal-derived food-protein of interest is an egg protein, i.e. a protein that is present in a bird’s egg. The term “bird” as used herein includes both domesticated birds and non-domesticated birds such as wild life birds. Birds e.g. include poultry, fowl, waterfowl, game bird, ratite (e.g., flightless bird), chicken ( Gallus gallus domesticus), quail, turkey, duck, ostrich ( Struthio camelus), Somali ostrich ( Struthio molybdophanes), goose, gull, guinea fowl, pheasant, emu ( Dromaius novaehollandiae), American rhea ( Rhea americana), Darwin’s rhea ( Rhea pennata) and kiwi. Preferably, the animal-derived food-protein of interest is an egg white protein. The egg white protein can be an egg white protein selected from the group consisting of ovalbumin, ovotransferrin, ovomucoid, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X and ovalbumin related protein Y (see e.g. US2018/0355020). A particularly preferred egg white protein is ovalbumin.

### Ovalbumin

A particularly preferred animal-derived food-protein of interest to be produced in an animal-free manner in accordance with the invention is the egg white protein ovalbumin.

In one embodiment, the ovalbumin that is encoded in the expression cassette of the invention comprises an amino acid sequence with at least 50, 55, 60, 65, 70, 75, 80, 85, 90, 95, 96, 97, 98, 99 or 100% identity to the amino acid sequence of an ovalbumin from a bird selected from the group consisting of chicken, pelican, quail, pigeon, ostrich, plover, turkey, duck, goose, gull, guinea fowl, jungle fowl, peafowl, partridge, pheasant, emu, rhea and kiwi, of which chicken, pigeon, pelican and quail are preferred.

In one embodiment, the ovalbumin that is encoded in the expression cassette of the invention comprises an amino acid sequence with at least 50, 55, 60, 65, 70, 75, 80, 85, 90, 95, 96, 97, 98, 99 or 100% identity to at least one of SEQ ID NO.′s: 1 - 6.

Alternative ovalbumin sequences from edible birds include: Genbank accession number AAC16664.1 and POI27989.1, for turkey and partridge, respectively. The examples of duck, goose, guinea fowl, pheasant, emu and kiwi ovalbumin sequences can be found under the NCBI reference number NP_001298098.1, XP_013056574.1, XP_021241976.1, XP_031445133.1, XP_025956522.1 and XP_025932497.1, respectively. In one embodiment, the ovalbumin may comprise amino acid sequences from more than one species.

In one embodiment, the amino acid sequence of the ovalbumin is modified so as to reduce or eliminate allergenicity. The allergenicity of an ovalbumin, e.g. chicken ovalbumin, can be reduced by replacing one or more amino acids in allergenic epitopes in the allergenic ovalbumin with different amino acid that are present in corresponding positions in the sequences of one or more ovalbumins from other bird species that are not allergenic. Methods used to make an assessment of allergenicity include but are not limited to initial bioinformatics techniques to later challenge testing.

### Fermentation

A further aspect of the invention relates to a process for producing an animal-derived food-protein of interest. The process preferably comprising the steps of: a) culturing the fungal host cell as herein defined in a medium in a fermenter under condition conducive to the expression of the protein of interest; and, b) optionally, recovery of the protein of interest.

The fermentation of fungal cells of the invention may be carried out in a stirred tank reactor. The reason for this choice by most of the industrial submerged fermentations of fungi lies on the rheology of (filamentous) fungal biomass in submerged cultures, which is affected, amongst others, by the morphology of the growing fungal mycelium. Consequently, in one embodiment the fermentation process of the invention is carried out in a stirred tank reactor. In a preferred embodiment however, the mechanical power into the medium in the fermenter is limited in order to prevent or reduce precipitation and/or aggregation in the medium of the protein of interest, in particular of an ovalbumin. In one embodiment therefore, the input of mechanical power (during the culturing of the fungal host) into the medium in the fermenter is no more than 1.4, 1.0, 0.5, 0.2 or 0.1 kW/m³.

Alternatively, or in addition to the use of (limited) mechanical power, a bubble column may be employed for the process of the invention. In general, the growth of fungi, in particular the mycelial growth of filamentous fungi, can lead to a highly viscous solution, which can negatively affect the aeration of the medium in the fermenter and thereby the growth and product formation. In order to obtain good oxygen and nutrient transfer of the viscous medium within the reactor, a high-power input for stirring is needed, which can give rise to precipitation and/or aggregation in the medium of the protein of interest to be produced, in particular in the case of an ovalbumin. However, strains of filamentous fungi, which are altered in their morphology, for instance those, which mycelial growth could be described as “yeast like morphology”, e.g. short hyphae and little of branching, could be also fermented in a reactor equipped with a bubble column (see for instance van ‘t Riet and Tramper, 1991 for description of type of bioreactors). Consequently, in one embodiment the fermentation is carried out in a bubble column, preferably without the input of mechanical power into the medium in the fermenter (e.g. for stirring). In this instance, the cultured medium is “stirred” or mixed by the gas (e.g. air or oxygen) bubbles moving upwards through the medium. In one embodiment the fermentation is carried out in a combination of a stirred tank reactor and a bubble column. In a further embodiment, the bubble column makes use of a batch mode, a fed batch mode or a repeated fed batch mode. In one embodiment, a high cell density fermentation is applied, where the operation may be carried out at packed cell volume of more than 10% (cells/volume) and even more then 25%, 30%, 40%, or sometimes even at 45% packed cell volume. In one embodiment, the bubble column is executed with a gas volume of > 0.5 vvm (broth volume replacement per minute), even higher than 0.7 vvm and most preferably at 1.0 vvm. The air provides good mixing of the fermenter and aeration. Therefore, in one embodiment the bubbles are sparged through holes in a pipe of 4 to 6 mm.

Media for growth of fungal host cells of the invention are generally known in the art. In a preferred embodiment, the medium for culturing a fungal host cell of the invention is a chemically defined medium. Typical composition of the chemically defined media for growth of (filamentous) fungi are e.g. described in US 20140342396 A1, incorporated by reference herein. The pH in the fermenter may be controlled using ammonia as titrant.

The process preferably uses a carbon source comprising at least one of glucose, sucrose, isomaltose and maltodextrines. The carbon source may be delivered in the form of thick juice (an intermediate of sugar beet processing) or a syrup comprising isomaltose and maltodextrines, such as a 95 DE syrup. One example with glucose includes a 30-95 DE syrup comprising, the maltose isomaltose as inducing compound of the glucoamylase promoter.

In one embodiment, the pH of the fermentation may be maintained between 3 to 8 pH, most likely between 4 to 7 pH. Depending on the type of the expressed protein, however, this range may be further fine-tuned. For example, the optimal pH forthe production of homologous enzymes using A. niger is pH 3.5 - 5.5. In the case of heterologous expression of animal proteins a higher pH range is preferred. E.g. for ovalbumin, having the pl value around 4.8 - 5, a pH of 5 to 7 pH is preferred. Furthermore, by monitoring the pH during the fermentation the induction spectrum of proteases can be altered. Consequently, in one embodiment, the fungal host cell is cultured at a pH that is equal to or higher than pH 5.0, 5.5, 6.0, 6.5 or 7.

### Processing

In one embodiment, the protein of interest is produced as a secreted protein, which, by virtue of the presence of the secretory signal sequence in the expression cassette, is secreted from the fungal host cell into the fermentation medium. Such secreted proteins of interest can be recovered from the fermentation broth in different ways. Therefore, in one embodiment, the method optionally includes step b) recovery of the albumen. In one embodiment, the recovery is during fermentation. In one embodiment, the recovery is post fermentation. In one embodiment, the recovery is both during and post fermentation. The recovery of the protein of interest preferably at least includes separation of the fungal biomass from the medium comprising the (dissolved) protein of interest. One of the possibilities to separate the microbial biomass is by centrifugation. Even more preferred the fermented broth may be set for release of the protein at the end of the fermentation, which may be following the separation of the released protein directly by centrifugation of the biomass. Therefore, in one embodiment, the recovery is by centrifugation. After the separation by centrifugation the supernatant usually contains most of the secreted protein. Consequently, in a further embodiment, the supernatant may require further processing. In one embodiment, the supernatant may be micro-filtrated using a 0.2 µm filter. Such practice will be known to one skilled in the art to remove remaining fragments of fungal biomass. In addition the supernatant can be ultra-filtrated through a membrane with variable molecular weight cut-off values, typically 10 kDa or 20 kDa, to further separate small molecular weight components from the ultrafiltrate. In one embodiment, such further processing includes ultrafiltration, wherein the supernatant is also concentrated. However, depending on the nature of the protein, hydrophobic proteins usually have the tendency to stick to the biomass. Such an example may be for instance found in US 8703463 B2, which describes expression of a lipase in a yeast Kluyveromyces lactis and a filamentous fungus Aspergillus niger. In this example, the protein was released from the biomass by changing the pH of the solution.

Generally, in order to release the secreted protein from the harvested biomass, several approaches may be tested. In one embodiment, the harvested biomass may be re-suspended. In one embodiment, the re-suspension is in the tested solution, for instance, at ratios of 1:1, 1:2, 1:3, 1:4 (biomass:solution). In one embodiment, the harvested biomass is incubated at set temperature for a certain period of time. Optionally, the solution may be stirred. In order to liberate the protein from the harvested biomass alternative methods are available are known to the skilled person. It will be known that one method may be employed or a combination of methods may be employed. In one embodiment, to liberate the protein from the biomass the method comprises: i) increasing or decreasing the pH of the solution to alter the charge of the exposed surface of the protein with consequently change the solubility of the protein. In one embodiment, the solution is increased at least 1 pH unit difference from isoelectric point of the protein (pl), which is the pH at which the protein has zero net charge.

For example, for a protein with pl value around 5 the 50 mM potassium phosphate buffer of 6 to 10 pH may be used. In one embodiment, to liberate the protein from the biomass the method comprises: i) using different concentration of salts to affect the interaction of the protein with the biomass. For example, NaCl may be used in the range of 0.1 to 1 M or 50 mM potassium phosphate buffer, with a different concentration of ammonium sulphate, for example 0.2- to 2 M. In one embodiment, to liberate the protein from the biomass the method comprises; i) use of non-ionic surfactants. For example, Tween 20.

Optionally, after microfiltration and ultrafiltration of the fermentation broth containing the expressed protein, the next step may involve purification. Alternatively, microfiltration and ultrafiltration may not be required. The choice of a purification method may influence the protein stability, purity and yield, but the costs, sustainability and waste streams associated with different methods differ. One of the most important criteria during purification is reduction of protein losses due to aggregation or denaturation. A person skilled in art will recognize that the following methods being typically applied for purification of proteins. In one embodiment, the purification is one or more of ammonium sulfate precipitation; an aqueous biphasic system (ABS); and chromatography methods. In a further embodiment, purification is chromatography selected form one or more of anion-exchange; cation-exchange; hydrophobic-interaction; and size-exclusion, chromatography.

Ammonium sulfate precipitation is a purification method based on the solubility of proteins in the presence of high salt concentrations, also known as the salting-out of proteins. The salt ions shield the charges on a protein molecule, allowing for interaction between molecules which can result in aggregation and precipitation of the proteins. The salt concentration at which this happens, depends on the structural properties of the native protein, which differs between proteins. In one embodiment, the protein solution is saturated to a certain percentage in a range of 1% of protein weight to the total weight (w/w % to 90 w/w % using a suitable salt. In one embodiment, the protein solution is stirred at the chosen saturation percentage at a certain temperature. In one embodiment the protein solution is at 0 to 10° C. for a duration of certain time. In one embodiment the precipitated protein may be collected through centrifugation. Such a method has been applied for purification of ovalbumin from egg-white. In one embodiment the purification process comprises a step using ammonium sulfate precipitation. In a preferred embodiment, the ammonium sulfate precipitation is used to purify ovalbumin from egg white. For example, in one embodiment ovalbumin is precipitated from egg white in a method comprising ammonium sulfate precipitation between a range of 50% to 90% saturation, such as 60% to 70% saturation. Said method may be applied during fermentation or during a purification step at the end of fermentation.

An aqueous biphasic system (ABS), when used for protein purification, may comprise a polyethylene glycol (PEG)-phase and a salt phase. This method has an advantage of being easily applicable on industrial large scale and it is low in costs when compared to other protein purification methods, such as chromatography. Furthermore, the PEG can be regenerated resulting in a low amount of waste streams. The principle of the purification is based on the interactions of the protein of interest with the PEG or salt (Asenjo, J.A & Andrews, B. A., (2011); Rito-Palomares. M., (2004)). The system has been used for the purification of ovalbumin from egg white. Such methods of using an ABS for the purification of egg-white and suitable alternatives would be known to one skilled in the art (Wen, C., et al. (2019); (Meihu, M., et al (2013)).

The different type of chromatographic methods applied in the protein purification steps, differ in their nature with regards to the protein interaction with the resin. Ion exchange chromatography (anion or cation) uses the charge of a protein (negative or positive, respectively) to separate it from the differently charged contaminants in a solution (DNA, other proteins, viruses etc.). For anion exchange chromatography, the anion exchange agent is positively charged. It can be either a weak anion exchange agent, composed of e.g. diethylaminomethyl (DEAE) groups, or a strong anion exchange agent, composed of e.g. quaternary ammonium (Q) groups. The strong anion exchangers are stable over the whole pH range, while the weak anion exchangers have pH limitations. To bind the protein of interest to the anion exchange agent, the pH of the buffer used (potassium phosphate, Tris-HCI, etc.) should be at least one pH value above the iso-electric point of the protein of interest, to obtain a negatively net charged protein. Subsequently, the proteins are eluted from the column based on the strength of their interaction with the anion exchange agent using an appropriate salt (sodium chloride, ammonium sulfate, etc.) increasing in molarity, or by decreasing the pH of the buffer (Haq, A., et al., (1999); Medve, J., et al (1998); Kopacieqicz, W., et al. (1983); Rossomando, E. F., (1990); Gooding, K. M., & Schmuck, M. N., (1985)). The principle of cation exchange chromatography is like anion exchange chromatography, except for using negatively charged instead of positively charged resins. This can be a weak cation exchange agent, composed of e.g. carboxymethylcellulose (CM-cellulose) groups, or a strong cation exchange agent, composed of e.g. sulphopropyl (SP) groups. For binding a protein to the cation exchange agent, the pH of the buffer should be at least 1 pH value below the iso-electric point of the protein, to obtain a positively charged protein. The loading and elution of the protein is similar as described for the anion exchange chromatography. Hydrophobic-interaction chromatography explores hydrophobicity as the basis of interaction between the protein and the resin. Protein binding happens under increased salt concentration (usually ammonium sulfate), to stimulate the exposure of the hydrophobic patches of the protein and the elution is done under decreasing salt concentrations (Soni, B., et al., (2008); Shaltiel, s., (1974); McCue, J. T., (2009); Queiroz, J. A., et al., (2001); Gooding, D. L., et al., (1986); Watanabe, E., et al., (1994) Narhi, L. Lo., et al., (1989)). Such methods of using a chromatographic methods forthe purification of egg-white and suitable alternatives would be known to one skilled in the art (Awade, A. C., et al., (1994); Guerin-Dubiard, C., et al., (2005); Croguennec, T., et al., (2000); Gen, F., et al., (2012)). When using size-exclusion chromatography for the purification of the protein of interest, the proteins are separated based on the hydrodynamic volume. The proteins pass through a column filled with beads with different pore sizes. The smaller proteins will fill the pores more easily than the larger proteins, resulting in a shorter elution time for larger proteins and a longer elution time for smaller proteins. The porous beads in the column can be agarose, dextran or polyacrylamide beads. The proteins are usually eluted isocratic with an appropriate buffer (e.g. phosphate) containing a low salt concentration (e.g. sodium chloride) (Mori, S., & Barth, H. G., (2013); Andre, A. S. A. H.-K., & Schwarm, K. S., (2016); Luo, J., et al., (2013)).

In one aspect, the invention pertains to a process for purifying an animal-derived food-protein of interest. The animal-derived food-protein of interest can be a protein as herein defined, such as an ovalbumin. In one embodiment, the animal-derived food-protein of interest is purified from a culture medium. In one embodiment, the culture medium is a culture medium in which a microbial host (cell) has been cultured during the production of the animal-derived food-protein of interest. The culture medium can thus be a spent culture medium of a microbial host (cell) wherein the protein has been expressed. In one embodiment, the microbial host cell is a fungal host cell is herein defined, such as an Aspergillus, or Aspergillus niger host cell. In one embodiment, the animal-derived food-protein of interest is secreted from the microbial host cell into the culture medium. In which embodiment, preferably, the biomass of the microbial host cell in which the protein has been produced, is removed from the spent culture medium, preferably prior to any subsequent steps for purification of the animal-derived food-protein of interest is purified from the culture medium in which the protein was produced.

In one embodiment, the process for purifying an animal-derived food-protein of interest from a spent culture medium that was obtained in a process for producing the protein of interest as herein defined.

In one embodiment, the process for purifying an animal-derived food-protein of interest comprises at least a first anion exchange step.

The ion exchange resin can be prepared according to known methods. Typically, an equilibration buffer, which allows the resin to bind its counter ions, can be passed through the ion exchange resin prior to loading the sample or composition comprising the polypeptide and one or more contaminants onto the resin. Conveniently, the equilibration buffer can be the same as the loading buffer, but this is not required.

In one embodiment, the process for purifying an animal-derived food-protein of interest includes, providing an aqueous solution comprising the protein of interest, and subjecting the sample to at least a first anion exchange chromatography step, e.g., anion exchange chromatography described herein. For the anion exchange resin, the charged groups which are covalently attached to the matrix can be, for example, diethylaminoethyl (DEAE), quaternary aminoethyl (QAE), and/or quaternary ammonium (Q). In some embodiments, the anion exchange resin employed is a Q Sepharose column. The anion exchange chromatography can be performed using, e.g., Q SEPHAROSE™ Fast Flow, Q SEPHAROSE™ High Performance, Q SEPHAROSE™ XL, CAPTO™ Q, DEAE, TOYOPEARL GIGACAP™ Q, FRACTOGEL™ TMAE (trimethylaminoethyl, a quarternary ammonia resin), ESHMUNO™ Q, NUVIA™ Q, or UNOSPHERE™ Q. Other anion exchangers can be used within the scope of the invention, including but not limited to, quaternary amine resins or “Q-resins” (e.g., CAPTO™-Q, Q-SEPHAROSE™, QAE SEPHADEX™); diethylaminoethane (DEAE) resins (e.g., DEAE-TRISACRYL™, DEAE SEPHAROSE™, benzoylated naphthoylated DEAE, diethylaminoethyl SEPHACEL™); AMBERJET™ resins; AMBERLYST™ resins; AMBERLITE™ resins (e.g., AMBERLITE™ IRA-67, AMBERLITE™ strongly basic, AMBERLITE™ weakly basic), cholestyramine resin, ProPac™. resins (e.g., PROPAC™ SAX-10, PROPAC™ WAX-10, PROPAC™ WCX-10); TSK-GEL™ resins (e.g., TSKgel DEAE-NPR; TSKgel DEAE-5PW); and ACCLAIM™ resins.

In one embodiment, the anion exchange chromatography is performed using HiTrap Capto Q™ AEC (Cytiva).

Thus, in a first anion exchange step the medium comprising the animal-derived food-protein of interest is contacted with the anion exchange resin. In one embodiment, the anion exchange resin is contained in a chromatography column and the medium comprising the protein of interest is loaded or applied to the column.

In one embodiment, the medium comprising the protein of interest is contacted with the anion exchange resin at an ionic strength or salt concentration that is equivalent to no more than 100, 90, 85, 80, 75, 70, 65, 60, 55, 50, 45, 40, 35, 30, 25, 20, 15, 10, 5, 2 or 1 mM NaCl.

In one embodiment, the medium comprising the protein of interest is contacted with the anion exchange resin at a pH from about 6 to about 9, e.g., from about 7 to about 8.5, from about 7.5 to about 8.3 e.g., about 8. The loading buffer, washing buffer, and elution buffer can include one or more buffering agents. For example, the buffering agent can be TRIS, HEPES, MOPS, PIPES, SSC, MES, sodium phosphate, potassium phosphate, or a combination thereof. The concentration of the buffering agent is between about 1 mM and about 250 mM, e.g., between about 10 mM and about 100 mM, between about 15 mM and about 50 mM, between about 20 mM and 30 mM, e.g. about 1 mM, about 5 mM, about 10 mM, about 20 mM, about 25 mM, about 30 mM, about 40 mM, or about 50 mM.

Thus, in one embodiment, the medium comprising the protein of interest is adjusted to the above-mentioned ionic strength and pH for contact with the anion exchange resin by means known in the art per se, such as dialysis or diafiltration.

In one embodiment, the medium comprising the protein of interest can also be subjected to other pre-treatments before being contacted with the anion exchange resin such as at least one of microfiltration (for removing biomass) and ultrafiltration (for concentrating the protein of interest), as e.g. described above.

In some embodiments, subjecting the medium comprising the protein of interest to the anion exchange is performed at a temperature about 23° C. or less, about 18° C. or less, or about 16° C. or less, e.g., about 23° C., about 20° C., about 18° C., or about 16° C.

In one embodiment, the protein of interest is collected from the first anion exchange step in the unbound fraction, i.e. the fraction that is not bound to the ion exchange resin. Thus, when the first anion exchange step is performed on a chromatography column, the protein of interest can be collected in the flow-through. Optionally the flow-through can be combined with one or more washes and/or elutions at an ionic strength or salt concentration that is equivalent to no more than 100, 90, 85, 80, 75, 70, 65, 60, 55, 50, 45, 40, 35, 30, 25, 20, 15, 10, 5, 2 or 1 mM NaCl, and preferably at a pH from about 6 to about 9, e.g., from about 7 to about 8.5, from about 7.5 to about 8.3 e.g., about 8.

In one embodiment, the protein of interest as obtained from the first anion exchange step can be subjected to a second anion exchange step that is performed under substantially identical conditions as the anion exchange first step.

In one embodiment, the protein of interest as obtained from the one or two anion exchange steps can be subjected to at least one of desalting and freeze-drying using means and methods generally known by the skilled person.

The animal-derived food-protein of interest to be anion exchange-purified from the spent medium in accordance with this aspect of the invention can be any animal-derived food-protein of interest described herein. Preferably, the protein of interest to be purified is an ovalbumin, more preferably an ovalbumin from a bird selected from the group consisting of chicken, pelican, quail, pigeon, ostrich, plover, turkey, duck, goose, gull, guinea fowl, jungle fowl, peafowl, partridge, pheasant, emu, rhea and kiwi, of which chicken, pigeon, pelican and quail are most preferred.

### Albumen

The microbially produced animal proteins, such as ovalbumin, which may be referred to as expressed ovalbumin, albumen or egg white, preferably exhibit functional properties that are comparable to those of the corresponding animal-derived proteins, in order to be able to use them in a similar way in food applications. In one embodiment, the expressed ovalbumin is part of a composition. Therefore, one may term the expressed ovalbumin as an albumen composition or an egg white composition. Alternatively, one skilled in the art may refer to the expressed ovalbumin as fungal derived expressed ovalbumin, fungal derived albumen or fungal derived egg white.

In order to predict the behavior of a microbially produced animal protein of the invention in complex applications such as baking, gelation etc., lab-scale experiments are applied, facilitating downscaling of the food application trials to a reasonable size and allowing to perform measurements and comparisons with standards. Consequently, the protein of the invention may be characterized. The term “albumen” as used herein, will be understood to describe one or more egg white proteins or egg white associated proteins, such proteins include but are not limited to tenp, clusterin, CH21, VMO-1, vitellogenin, zona pellucida C protein, ovotransferrin BC type, ovoinhibitor precursor, ovomucoid precursor, clusterin precursor, Hep21 protein precursor, ovoglycoprotein precursor, extracellular fatty acid-binding protein, extracellular fatty acid-binding protein precursor, prostaglandin D2 syntmay havee brain precursor, marker protein, vitellogenin-1, vitellogenin-2, vitellogenin- 2 precursor, vitellogenin-3, riboflavin binding protein, hemopexin, serum albumin precursor, apolipoprotein D, ovosecretoglobulin, Hep21, glutathione peroxidase 3, lipocalin-type prostaglandin D syntmay havee/chondrogenesis-associated lipocalin, apovitellenin-1, dickkopf-related protein 3, gallinacin-11 (VMO-11, (I-defensin-II), serum albumin (a-livetin), gallin, secretory trypsin inhibitor, lymphocyte antigen 86, actin, Ig p chain C region, sulfhydryl oxidase 1, histone H4, angiopoietin-like protein 3, ubiquitin, ovocalyxin-32, polymeric immunoglobulin receptor, peptidyl-prolyl-cis/trans isomerase B, aminopeptidase Ey, pleiotrophin, midkine, renin/prorenin receptor, TIMP-2, TIMP-3, histone H2B variants, Ig X chain, FAMC3 protein, α-enolase, 60S acidic ribosomal protein PI, cytotactin/tenascin, CEPU-1, selenoprotein, elongation factor 1-a 1, epididymalsecretory protein, El, 14-3-3 Protein (zeta), olfactomedinlike protein 3, glutathione S-transferase 2, P-2-microglobulin, RGD-CAP, apolipoprotein B, golgi apparatus protein 1, cochlin, proteasome subunit a type-7, apolipoprotein A-I, eukaryotic initiation factor 4A-II, ASPIC/cartilage acidic protein 1, triosephosphate isomerase, proteasome subunit a-type, Ig X chain C-region, procollagen-lysine 2-oxoglutarate 5-dioxgenase 1, ADP-ribosylation factor 5, calmodulin, protein disulfide-isomerase, annexin I, elongation factor 2, peroxiredoxin-1, HSP70, protein disulfide isomerase A3, calreticulin, 40S ribosomal protein SA/laminin receptor 1, a-Actinin-4, tumor necrosis factor-related apoptosis-inducing ligand, vitamin D-binding protein, semaphorin-3C, endoplasmin, catalase, hepatic a-amylase, transitional ER ATPase, cadherin-1, angiotensin-converting enzyme, bone morphogenetic protein 1, guanine nucleotide-binding protein subunit (12-like 1, histidine ammonia lyase, annexin A2, (1-catenin, RAB-GDP dissociation inhibitor, lamin-A, ovocleidin-116, aminopeptidase, HSP90-a, hypoxia up-regulated protein 1, heat shock cognate protein HSP90 (1, ATPcitrate syntmay havee, and myosin-9.

To determine the gelling properties of a protein of interest, which may be referred to as ovalbumin, albumen or egg white, an oscillatory rheometer (e.g. MCR series, Anton Paar) or a tensile tester may be used for obtaining the gel rheological characteristics, such as the storage modulus, loss modulus, gel strength, breaking stress, deformation, young modules. Typically, to obtain elaborate information on the proteins gelling properties, the skilled person would accommodate gels that are being prepared varying the protein and salt concentration (Kato, A., et al., (2013); Hua, Y., et al., (2005); Rawdkuen, S., et al., (2009), such as for ovalbumin gel (Egelandsdal, B., (1980); Shitamori, S., etal., (1984); Matsudomi, N., etal., (1991); Hatta, H., etal., (1986); Shigeru, H., & Shuryo, N., (1985); Creusot, N., et al., (2011)). In such examples, the protein concentration may range between 3% and 20%, such as preferably between 5% and 15%. The salt (e.g. sodium chloride) may range between 0 and 200 mM, such as between 20 and 100 mM. The pH of the solution may range between 3 and 9, such as between 5 and 8, or between 6.5 and 7.5. The solution may be heated for a time range of 15 to 120 minutes, preferably 45 to 120 minutes, such as 60 to 90 minutes. In one example, the solution may be heated between 60 to 100° C., such as between 70 to 90° C. Hereafter, the gel is cooled to room temperature and ready to undergo rheological measurements.

In any one of the preceding embodiments, the albumen composition may have a gel strength within the range from 100 g to 1500 g, from 500 g to 1500 g, or from 700 g to 1500 g. In any one of the preceding embodiments, the albumen composition may have a gel strength greater than a gel strength of an albumen.

Another important quality of a protein is its solubility. In one embodiment, protein solubilization is determined by mixing different wt% (weight %) of a protein with buffer solutions of varying pH. In a further embodiment, the protein solution is stirred for a certain time and then centrifuged. The amount of protein in the supernatant may be measured using a suitable protein determination method, such as Bradford, Dumas, absorbance at 280 nm, Biuret, etc. for measurement of the protein solubility.

In any one of the preceding embodiments, the albumen composition may have a pH within the range from 6 to 10.

Another important food protein application is foaming. To determine the foaming properties of the protein of interest, a protein solution is prepared and may be agitated with a suitable method for a certain amount of time, for example homogenization or blending. The total foam volume after 30 s is observed using a measuring cylinder and defines the foam capacity. The total foam volume may be recorded over a period ranging from 1 to 24 h and the rate (volume %) at which the foam volume decreases, defines the foam stability. The foam stability may be expressed as half-life which in this case, refers to half the time needed for half of the foam to disappear.

In any one of the preceding embodiments, the egg white protein composition may have a foam height of at least 30 mm. In any one of the preceding embodiments, the egg white protein composition may have a foam height greater than a foam height of an egg white. In any one of the preceding embodiments, the egg white protein composition may have a foam seep up to 10 mm or up to 5 mm at 30 minutes after whipping. In any one of the preceding embodiments, the egg white protein composition may have a foam seep less than a foam seep of an egg white at 30 minutes after whipping. In any one of the preceding embodiments, the egg white protein composition may have a foam strength within the range from 30 g to 100 g, such as from 40 g to 100 g. In any one of the preceding embodiments, the egg white protein composition may have a foam strength greater than a foam strength of an egg white.

In any one of the preceding embodiments, the albumen composition may have a foam height greater than a foam height of an egg white. In any one of the preceding embodiments, the albumen composition may have a foam seep up to 10 mm or up to 5 mm at 30 minutes after whipping. In any one of the preceding embodiments, the albumen composition may have a foam seep less than a foam seep of an egg white at 30 minutes after whipping. In any one of the preceding embodiments, the albumen composition may have a foam strength within the range from 30 g to 100 g, such as from 40 g to 100 g. In any one of the preceding embodiments, the albumen composition may have a foam strength greater than a foam strength of albumen.

A further important functional property of a protein is its emulsifying properties. To measure this property, a protein solution with a certain pH may be mixed with a suitable oil. This solution is agitated with a suitable method, for example homogenization or blending, for a period of time. Alternative methods for determining the emulsifying properties are known in the art. One such method is based on the volumes of the emulsified phase, water phase and oil phase. A further method is based on the turbidity of the emulsion, which may be measured using a spectrophotometer.

In any one of the preceding embodiments, the ovalbumin composition may have a pH within the range from 6 to 10.

### Albumen Composition

In any one of the preceding embodiments, the ovalbumin expressed may be part of a composition. The ovalbumin expressed may also be known as albumen, as described herein. In any one of the preceding embodiments, the method may further comprise adding a food additive to the ovalbumin composition, also known as the albumen composition. In one aspect, the present disclosure provides a processed consumable product comprising the expressed ovalbumin or ovalbumin as part of a composition thereof. Consequently, the present disclosure provides a processed consumable product comprising albumen described herein. In one aspect, the present disclosure provides a processed consumable product comprising the expressed ovalbumin and one or more egg white proteins or fragments thereof. Therefore, the present disclosure provides a processed consumable product comprising the expressed ovalbumin and one or more albumen associated proteins or fragments thereof, as described herein. In some embodiments, the one or more egg white proteins may be selected from the group consisting of ovotransferrin, ovomucoid, G162M F167A ovomucoid, ovoglobulin G2, ovoglobulin G3, a-ovomucin, (3-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof. In any one of the preceding embodiments, the processed consumable product may comprise ovalbumin and two or more, three or more, four or more, five or more, or six or more egg white proteins or fragments thereof. In any one of the preceding embodiments, the processed consumable product may lack one or more, two or more, three or more, five or more, ten or more, or twenty or more egg white proteins. In any one of the preceding embodiments, the processed consumable product may be selected from the group consisting of food product, beverage product, dietary supplement, food additive, pharmaceutical product, hygiene product, and any combination thereof, such as from the group consisting of food product and beverage product. In any one of the preceding embodiments, the ovalbumin composition or consumable product may further comprise water. In any one of the preceding embodiments, the ovalbumin composition or consumable product may have a percentage of water up to 95%. In any one of the preceding embodiments, the ovalbumin composition or consumable product may have a percentage of water within the range from 80% to 95%. In any one of the preceding embodiments, the ovalbumin composition or consumable product may comprise at least 90% protein by dry weight. In any one of the preceding embodiments, the ovalbumin composition or consumable product may further comprise a food additive. In any one of the preceding embodiments, the food additive may be selected from the group consisting of a sweetener, salt, carbohydrate, and any combination thereof. In any one of the preceding embodiments, the ovalbumin composition or consumable product may lack cholesterol. In any one of the preceding embodiments, the ovalbumin composition or consumable product may comprise less than 5% fat by dry weight. In anyone of the preceding embodiments, the ovalbumin composition or consumable product may lack fat, saturated fat, or trans-fat. In any one of the preceding embodiments, the ovalbumin composition or consumable product may lack glucose.

In any one of the preceding embodiments, the method may further comprise desugaring, stabilizing, or removing glucose from the ovalbumin composition. In any one of the preceding embodiments, the method may further comprise pasteurizing or ultrapasteurizing the ovalbumin composition. In any one of the preceding embodiments, the method may further comprise drying the ovalbumin composition. In any one of the preceding embodiments, the method may further comprise enzymatically, chemically, or mechanically digesting the ovalbumin composition, or part thereof. In any one of the preceding embodiments, the albumen composition may have a shelf life of at least one, two, three, or six months. In any one of the preceding embodiments, the albumen composition or consumable product may have reduced allergenicity relative to albumen. In any one of the preceding embodiments, the albumen composition or consumable product may be a liquid. In any one of the preceding embodiments, the albumen composition or consumable product may be a solid or powder. In any one of the preceding embodiments, the albumen composition or consumable product may be frozen. In any one of the preceding embodiments, the albumen composition or consumable product may comprise some or all native A. niger proteins. Alternatively, the albumen composition or consumable product may be purified of all native A. niger proteins.

All patent and literature references cited in the present specification are hereby incorporated by reference in their entirety.

The present invention is further described by the following examples which should not be construed as limiting the scope of the invention.

### DESCRIPTION OF THE FIGURES

FIG. 1 Ovalbumin amino acids sequence alignment of selected edible birds. In the hen ovalbumin sequence the allergenic epitopes are depicted by boxes. Boxes marked 1 indicate IgE allergenic epitopes (Mine and Rupa, 2003); boxes marked 2 indicate epitopes from IEDB and SEDB databases; and, the box marked 3 indicates basophil induction epitopes (Honma et al., 1996).

FIG. 2 Codon usage table of A. niger based on the whole genome.

FIG. 3 SDS-PAGE example of selected ovalbumin A. niger transformants fermented in 96-well deep well plate (DWP) or shake flask (stated per sample from which source). OVA stock corresponds to the ovalbumin standard (Invivogen). 5 µl BenchMark™ Unstained Protein Ladder were loaded on the gel to determine the Mw of the protein bands.

A) Lanes contain the A. niger wild type, the A. niger OVA transformants carrying different expression constructs and the OVA standard as follows : 1. BZASNI.22a (shake flask); 2. GLA502 carrier OVA hen (CO glaA) with DDDK site (shake flask); 3. OVA hen (CO glaA, DWP); 4. OVA hen (CO whole genome, DWP); 5. OVA Pelican (CO whole genome, DWP); 6. OVA Quail (CO whole genome, DWP); 7. BZASNI.48 (shake flask); 8. OVA hen (CO glaA, DWP); 9. OVA Pelican (CO glaA, DWP); 10. OVA standard (0.05 g/l); 11. OVA standard (0.1 g/l); and 12. OVA standard (0.3 g/l). Samples in lanes 2 to 6 were produced in a BZASNI.22a background and sample in lanes 8 and 9 in a BZASNI.48 background. OVA stands for ovalbumin, CO for codon optimized.

B) Lanes contain the A. niger wild type, the A. niger OVA transformants carrying different expression constructs and the OVA standard as follows: 1. BZASNI.22a (shake flask); 2. GLA502 carrier OVA hen (CO glaA, shake flask) with DDDK site; 3. OVA hen (CO glaA, DWP); 4. BZASNI.48 (shake flask); 5. OVA hen (CO glaA, shake flask); 6. GLA54 OVA hen (CO glaA, shake flask) transformant 1; 7. GLA54 OVA hen (CO glaA, shake flask) transformant 2; 8. GLA100 OVA hen (CO glaA, shake flask) transformant 1; 9. GLA100 OVA hen (CO glaA, shake flask) transformant 2; 10. GLA502 OVA hen (CO glaA, shake flask) transformant 1; 11. GLA502 OVA hen (CO glaA, shake flask) transformant 2; 12. OVA standard (0.05 g/l); 13. OVA standard (0.1 g/l); and 14. OVA standard (0.3 g/l). Samples in lanes 2 and 3 were produced in a BZASNI.22a background and sample in lanes 6 to 11 in a BZASNI.48 background.

FIG. 4 The overexpression of hen ovalbumin in A. niger BZESCO.22 (#25 & #26) and BZESCO.23 (B20) transformants detected by Western blot using hen ovalbumin polyclonal antibodies in supernatant of day 5 (d5) and day 6 (d6) DWP (deep well plate) cultures. The left panel correspond to the SDS-PAGE gel and the right panel to the Western blot of an identical SDS-PAGE gel as shown in the left. The most right arrow in each panel indicates the position of the standard (monomer) ovalbumin (Invivogen) and the two arrows found above each other the overexpressed hen ovalbumin in A.niger.

FIG. 5. SDS-PAGE of filtrated (0.2 µ) of fermentation samples of the supernatant of A.niger BZASNI.33. S1-S5 correspond to time of 0.5, 17.5, 32, 41, 47.5 hrs of the fermentation in 5 L fermenter, respectively. The arrow depicts the secreted hen ovalbumin. Ovalbumin standard (left in the FIG. 5) was purchased from Invivogen.

FIG. 6. Purification of hen ovalbumin produced in A. niger BZASNI.60 in shake flask. (A) First step of purification on Anion Exchange Column. (B) the second step purification on Anion Exchange column using the flow-through fraction obtained from the first step, containing ovalbumin.

### EXAMPLES

### General Molecular Biology Techniques

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

### Strains

Aspergillus niger strain BZASNI.22a is a faster growing single colony isolate of CICC2462. BZASNI.22a was characterized by rDNA ITS sequencing and by resequencing of the genome (BaseClear, the Netherlands). Strain A. niger BZASNI.33 is a high-copy transformant of A. niger BZASNI.22a with the GLA carrier-OVA expression cassette (cloned in BZESCO.23).

BZASNI.48 is a single colony isolate of BZASNI.22a. BZASNI.60 is a high-copy transformant of A. niger BZASNI.48 with the GLA pre-pro-peptide OVA expression cassette.

For cloning purposes NEB 10-beta Competent Escherichia coli ( E. coli) cells and NEB 5-alpha Competent E. coli cells from New England Biolabs were used, which were are obtained from Bioke.

Alternative strains include, A. niger CBS513.88 (wild type), the NRRL3 strain (wild type) and the A. niger CICC2462.

### Plasmids and Oligonucleotide Primers

Plasmids used in the Examples are listed in Table 1. Primers used in the Examples are listed in Table 2.

Table 1

Listing of plasmids used in examples

| | | |
|---|---|---|
| Name | Characteristics | Origin and/or SEQ ID NO |
| pUC57-kan | neomycin/kanamycin resistance marker, E. coli plasmid | delivery plasmid for all synthetic genes, from Genscript (SEQ ID NO. 13) |
| pCR™Blunt ll-TOPO® | neomycin/kanamycin resistance marker, E. coli plasmid | plasmid for ligation purified PCR products, from Zero Blunt™ TOPO™ PCR Cloning Kit (SEQ ID NO. 21) |
| pGGA | chloramphenicol resistance marker, E. coli plasmid | Golden Gate vector, from NEB Golden Gate Assembly Kit (SEQ ID NO. 22) |
| pBZ0024 | vector with glaAp (from A. niger) | SEQ ID 23 |
| pBZ0025 | vector with glaAt (from A. niger) | SEQ ID 24 |
| pBZ0028 | vector with cassette glaAp -OVA (Ostrich) - glaAt | SEQ ID 25 |
| pBZ0029 | Vector with cassette glaAp -OVA (Plover) - glaAt | SEQ ID 26 |
| pBZ0030 | vector with cassette glaAp -OVA (Pelican) - glaAt | SEQ ID 27 |
| pBZ0031 | vector with cassette glaAp -OVA (Pigeon) - glaAt | SEQ ID 28 |
| pBZ0032 | vector with cassette glaAp -OVA (Quail) - glaAt | SEQ ID 29 |
| pUC57 | Ampicillin resistance marker, E. coli plasmid | delivery plasmid for synthetic genes, from Genscript (SEQ ID NO. 31) |
| FXESCO.12 | Hen ovalbumin (codon optimized with codon usage of A. niger glaA gene) in pUC57 | SEQ ID 32 |
| pUC57-Brick | Ampicillin resistance marker, E. coli plasmid | delivery plasmid for synthetic genes, from Genscript (SEQ ID NO. 42) |
| FXESCO.14 | Hen OVA construct (codon usage glaA) in pUC57-Brick | SEQ ID 43 |
| FXESCO.15 | GLA carrier fusion + Hen OVA construct (codon usage glaA) in pUC57-Brick | SEQ ID 44 |
| pCNS43 | hygromycin selection marker between Aspergillus nidulans tryptophan C promoter and terminator (trpC) | ordered at BCCM Belgian Coordinated Collections of Microorganisms (SEQ ID NO. 45) |
| pBZ0026 | Aspergillus nidulans gpdA promoter, amdS ORF and trpC terminator | SEQ ID NO. 62 |
| pBZ0041 | vector with cassette glaAp -OVA (Ostrich, CPO glaA) -glaAt | SEQ ID NO. 70 |
| pBZ0042 | glaAp - OVA (Plover, CPO glaA) - glaAt | SEQ ID NO. 71 |
| pBZ0043 | glaAp - OVA (Pelican, CPO glaA) - glaAt | SEQ ID NO. 72 |
| pBZ0044 | glaAp - OVA (Pigeon, CPO glaA) - glaAt | SEQ ID NO. 73 |
| pBZ0045 | glaAp - OVA (Quail, CPO glaA) - glaAt | SEQ ID NO. 74 |
| pUC19 | ampicillin resistance marker, E. coli plasmid | Cloning vector from New England Bioloabs; SEQ ID NO. 79 |
| pBZ0061 | pUC19 - 3″glaA - glaAp - OVA (hen, FXESCO.14) - glaAt | SEQ ID NO. 82 |
| pBZ0062 | pUC19 - 3″glaA - glaAp - OVA (Ostrich, CPO glaA) - glaAt | SEQ ID NO. 83 |
| pBZ0063 | pUC19 - 3″glaA - glaAp - OVA (Plover, CPO glaA) - glaAt | SEQ ID NO. 84 |
| pBZ0064 | pUC19 - 3″glaA - glaAp - OVA (Pelican, CPO glaA) - glaAt | SEQ ID NO. 85 |
| pBZ0065 | pUC19 - 3″glaA - glaAp - OVA (Pigeon, CPO glaA) - glaAt | SEQ ID NO. 86 |
| pBZ0066 | pUC19 - 3″glaA - glaAp - OVA (Quail, CPO glaA) - glaAt | SEQ ID NO. 87 |
| pBZ0046 | glaAp - GLA54 - OVA (Ostrich, CPO glaA) - glaAt | SEQ ID NO. 102 |
| pBZ0047 | glaAp - GLA54 - OVA (Plover, CPO glaA) - glaAt | SEQ ID NO. 103 |
| pBZ0048 | glaAp - GLA54 - OVA (Pelican, CPO glaA) - glaAt | SEQ ID NO. 104 |
| pBZ0049 | glaAp - GLA54 - OVA (Pigeon, CPO glaA) - glaAt | SEQ ID NO. 105 |
| pBZ0050 | glaAp - GLA54 - OVA (Quail, CPO glaA) - glaAt | SEQ ID NO. 106 |
| pBZ0051 | glaAp - GLA100 - OVA (Ostrich, CPO glaA) - glaAt | SEQ ID NO. 107 |
| pBZ0052 | glaAp - GLA100 - OVA (Plover, CPO glaA) - glaAt | SEQ ID NO. 108 |
| pBZ0053 | glaAp - GLA100 - OVA (Pelican, CPO glaA) - glaAt | SEQ ID NO. 109 |
| pBZ0054 | glaAp - GLA100 - OVA (Pigeon, CPO glaA) - glaAt | SEQ ID NO. 110 |
| pBZ0055 | glaAp - GLA100 - OVA (Quail, CPO glaA) - glaAt | SEQ ID NO. 111 |
| pBZ0056 | glaAp - GLA502 - OVA (Ostrich, CPO glaA) - glaAt | SEQ ID NO. 112 |
| pBZ0057 | glaAp - GLA502 - OVA (Plover, CPO glaA) - glaAt | SEQ ID NO. 113 |
| pBZ0058 | glaAp - GLA502 - OVA (Pelican, CPO glaA) - glaAt | SEQ ID NO. 114 |
| pBZ0059 | glaAp - GLA502 - OVA (Pigeon, CPO glaA) - glaAt | SEQ ID NO. 115 |
| pBZ0060 | glaAp - GLA502 - OVA (Quail, CPO glaA) - glaAt | SEQ ID NO. 116 |
| pBZ0067 | pUC19 - 3″glaA - glaAp -GLA54 - OVA (hen, FXESCO.14) - glaAt | SEQ ID NO. 125 |
| pBZ0068 | pUC19 - 3″glaA - glaAp -GLA100 - OVA (hen, FXESCO.14) - glaAt | SEQ ID NO. 126 |
| pBZ0069 | pUC19 - 3″glaA - glaAp -GLA502 - OVA (hen, FXESCO.14) - glaAt | SEQ ID NO. 127 |

Table 2

Listing of oligonucleotide primers used in the examples with sequences

| Primer code | SEQ ID | Comment |
|---|---|---|
| 406 | SEQ ID NO. 17 | glaA promoter amplification, glaAp - OVA (all different) -glaAt amplification |
| 408 | SEQ ID NO. 18 | glaA promoter amplification |
| 409 | SEQ ID NO. 19 | glaA terminator amplification |
| 410 | SEQ ID NO. 20 | glaA terminator amplification, glaAp - OVA (all different) -glaAt amplification |
| 276 | SEQ ID NO. 34 | glaA promoter amplification |
| 286 | SEQ ID NO. 35 | glaA promoter amplification + truncated glaA |
| 277 | SEQ ID NO. 36 | glaA promoter amplification |
| 280 | SEQ ID NO. 37 | glaAt amplification |
| 281 | SEQ ID NO. 38 | glaAt amplification |
| 278 | SEQ ID NO. 39 | Hen OVA amplification |
| 279 | SEQ ID NO. 40 | Hen OVA amplification |
| 287 | SEQ ID NO. 41 | Hen OVA amplification |
| 404 | SEQ ID NO. 46 | hygromycin marker amplification |
| 405 | SEQ ID NO. 47 | hygromycin marker amplification |
| 553 | SEQ ID NO. 48 | diagnostic primer OVA ostrich |
| 554 | SEQ ID NO. 49 | diagnostic primer OVA ostrich |
| 555 | SEQ ID NO. 50 | diagnostic primer OVA plover |
| 556 | SEQ ID NO. 51 | diagnostic primer OVA plover |
| 557 | SEQ ID NO. 52 | diagnostic primer OVA pelican |
| 558 | SEQ ID NO. 53 | diagnostic primer OVA pelican |
| 559 | SEQ ID NO. 54 | diagnostic primer OVA pigeon |
| 560 | SEQ ID NO. 55 | diagnostic primer OVA pigeon |
| 561 | SEQ ID NO. 56 | diagnostic primer OVA quail |
| 562 | SEQ ID NO. 57 | diagnostic primer OVA quail |
| 336 | SEQ ID NO. 58 | diagnostic primer hygromycin |
| 337 | SEQ ID NO. 59 | diagnostic primer hygromycin |
| 400 | SEQ ID NO. 60 | diagnostic primer amdS |
| 401 | SEQ ID NO. 61 | diagnostic primer amdS |
| 411 | SEQ ID NO. 63 | amdS marker amplification |
| 415 | SEQ ID NO. 64 | amdS marker amplification |
| 728 | SEQ ID NO. 75 | 3″ glaA amplification |
| 729 | SEQ ID NO. 76 | 3″ glaA amplification |
| 733 | SEQ ID NO. 77 | glaA promoter amplification, glaAp - OVA (all different) -glaAt amplification |
| 734 | SEQ ID NO. 78 | glaA terminator amplification, glaAp - OVA (all different) -glaAt amplification |
| 431 | SEQ ID NO. 80 | Ampicillin + ori from pUC19 |
| 432 | SEQ ID NO. 81 | Ampicillin + ori from pUC19 |
| 715 | SEQ ID NO. 92 | glaA promoter amplification, glaAp - OVA Ostrich - glaAt amplification |
| 422 | SEQ ID NO.93 | glaA terminator amplification, glaAp - OVA Ostrich - glaAt amplification |
| 716 | SEQ ID NO. 94 | glaA promoter amplification, glaAp - OVA Plover - glaAt amplification |
| 424 | SEQ ID NO. 95 | glaA terminator amplification, glaAp - OVA Plover - glaAt amplification |
| 717 | SEQ ID NO. 96 | glaA promoter amplification, glaAp - OVA Pelican - glaAt amplification |
| 426 | SEQ ID NO. 97 | glaA terminator amplification, glaAp - OVA Pelican - glaAt amplification |
| 718 | SEQ ID NO. 98 | glaA promoter amplification, glaAp - OVA Pigeon - glaAt amplification |
| 428 | SEQ ID NO. 99 | glaA terminator amplification, glaAp - OVA Pigeon - glaAt amplification |
| 719 | SEQ ID NO. 100 | glaA promoter amplification, glaAp - OVA Quail - glaAt amplification |
| 430 | SEQ ID NO. 101 | glaA terminator amplification, glaAp - OVA Quail - glaAt amplification |
| 823 | SEQ ID NO. 117 | glaA promoter amplification, glaA - GLA54 amplification |
| 824 | SEQ ID NO. 118 | GLA54 amplification, glaA -GLA54 amplification |
| 825 | SEQ ID NO. 119 | GLA100 amplification, glaA -GLA100 amplification |
| 826 | SEQ ID NO. 120 | GLA502 amplification, glaA -GLA502 amplification |
| 827 | SEQ ID NO. 121 | GLA 54 - OVA hen amplification, OVA (hen, CPO glaA) - glaA terminator -backbone - 3″ glaA amplification |
| 828 | SEQ ID NO. 122 | GLA 100 - OVA hen amplification, OVA (hen, CPO glaA) - glaA terminator -backbone - 3″ glaA amplification |
| 829 | SEQ ID NO. 123 | GLA 502 - OVA hen amplification, OVA (hen, CPO glaA) - glaA terminator -backbone - 3″ glaA amplification |
| 830 | SEQ ID NO. 124 | 3″ glaA part amplification -OVA (hen, CPO glaA) - glaA terminator - backbone - 3″ glaA amplification |
| 787 | SEQ ID NO. 128 | 3″ glaA amplification for transformation, 3″ glaA -(GLA54/100/502) - glaA promoter - OVA (selected ovalbumins) - glaA terminator |
| 789 | SEQ ID NO. 129 | glaA terminator for transformation, 3″ glaA -(GLA54/100/502) - glaA promoter - OVA (selected ovalbumins) - glaA terminator |

### Kits

For the purification of PCR fragments and extraction of DNA fragments from agarose gel the Wizard® SV Gel and PCR clean-up system (Promega) was used. Purified PCR products were transformed in the pCR™Blunt II-TOPO® vector from the Zero Blunt™ TOPO™ PCR Cloning Kit (Thermo Fisher Scientific). Amplified plasmids were isolated with the Qiaprep spin miniprep kit (Qiagen). Golden Gate Reactions were carried out with the NEB Golden Gate Assembly Kit (New England Biolabs). Gibson Cloning Reactions were carried out with the NEB Gibson Assembly Cloning Kit (New England Biolabs).

### Enzymes

Enzymes for DNA manipulations (e.g. digestions, Golden Gate Reactions) were obtainable from New England Biolabs and were used according to the manufacturer’s protocols.

### Media

Media used in the construction of ovalbumin expression cassettes: liquid LB (10 g/l Tryptone, 5 g/l Yeast extract, 5 g/l NaCl) and solid LB (addition of 15 g/l agar) with the appropriate antibiotic (Neomycin 50 µg/ml or chloramphenicol 25 µg/ml).

Media used for transformation and selection of the A. niger transformants: Bottom agar (187.36 g/L D-Saccharose; 0.5 g/kg KCI; 4 g/kg KH2PO4; 1.1 g/kg Na2HPO4; 1.5 g/kg Citric acid; 2 g/kg MgSO4.7 aq; 0.01 g/kg FeSO4.7 aq; 0.1 g/kg CaCl2.2 aq; 0.0125 g/kg ZnSO4.7 aq; 0.012 g/kg MnCl2.4 aq; 0.0016 g/kg CuSO4.5 aq; 0.0009 g/kg KI, 9 g/L agarose and initial pH of 6) with the appropriate selection (antibiotic hygromycin 400 µg/ml or 10 mM acetamide in a combination with 15 mM caesium chloride), Top agar (0.5 g/kg KCI; 4 g/kg KH2PO4; 1.1 g/kg Na2HPO4; 1.5 g/kg Citric acid; 2 g/kg MgSO4.7 aq; 0.01 g/kg FeSO4.7 aq; 0.1 g/kg CaCl2.2 aq; 0.0125 g/kg ZnSO4.7 aq; 0.012 g/kg MnCl2.4 aq; 0.0016 g/kg CuSO4.5 aq; 0.0009 g/kg KI, 11 g/L D-glucose, 9 g/L agar and initial pH of 6) with the appropriate selection (antibiotic hygromycin 500 µg/ml or 10 mM acetamide in a combination with 15 mM caesium chloride) and minimal medium (0.5 g/kg KCI; 4 g/kg KH2PO4; 1.1 g/kg Na2HPO4; 1.5 g/kg Citric acid; 2 g/kg MgSO4.7 aq; 0.01 g/kg FeSO4.7 aq; 0.1 g/kg CaCl2.2 aq; 0.0125 g/kg ZnSO4.7 aq; 0.012 g/kg MnCl2.4 aq; 0.0016 g/kg CuSO4.5 aq; 0.0009 g/kg KI, 11 g/L D-glucose, 9 g/L agar and initial pH of 6) with the appropriate selection (antibiotic hygromycin 500 µg/ml or 10 mM acetamide in a combination with 15 mM caesium chloride). If hygromycine was used for the selection of transformants the transformation plates and the minimal plates were containing 0.54 g/L NH₄Cl as the nitrogen source. Sporulation agar (0.54 g/L NH₄Cl, 0.5 g/kg KCI; 4 g/kg KH2PO4; 1.1 g/kg Na2HPO4; 1.5 g/kg Citric acid; 2 g/kg MgSO4.7 aq; 0.01 g/kg FeSO4.7 aq; 0.1 g/kg CaCl2.2 aq; 0.0125 g/kg ZnSO4.7 aq; 0.012 g/kg MnCl2.4 aq; 0.0016 g/kg CuSO4.5 aq; 0.0009 g/kg KI , 11 g/L D-glucose, 14 g/L agar and 44.73 g/L KCL and initial pH of 6) was used to purify the A. niger transformants.

Media used for screening of ovalbumin transformants in deep-well plates: PDA (20 g/L dextrose, 15 g/L agar, and 4 g/L potato starch). Production medium — PM — (0.5 g/kg KCI; 4 g/kg KH2PO4; 1.1 g/kg Na2HPO4; 1.5 g/kg Citric acid; 2 g/kg MgSO4.7 aq; 0.01 g/kg FeSO4.7 aq; 0.1 g/kg CaCl2.2 aq; 0.0125 g/kg ZnSO4.7 aq; 0.012 g/kg MnCl2.4 aq; 0.0016 g/kg CuSO4.5 aq; 0.0009 g/kg KI, 88 g/L D-glucose, 70 g/L citric acid, 12.5 g/L L-glutamine, 11 g/L tri-ammonium citrate and initial pH of 5.25).

Media used for screening of ovalbumin transformants in shake flasks: PDA. PM. GYE medium (20 g/L yeast extract, 22 g/L D-glucose and initial pH of 5). KM3.0 (5 g/L urea, 50 g/L D-glucose, 2 g/L KH2PO4, 0.55 g/L Na2HPO4, 1 g/L MgSO4.7 aq, 0.000125 ZnSO4.7 aq, 0.0001 g/L FeSO4.7 aq, 0.006 g/L MnCl2.4 aq, 0.05 g/L CaCl2.2 aq, 0.3 g/l KCI, 1.5 g/L Citric acid and initial pH of 5).

Fermentation medium used in 5 L scale fermenters: the defined fermentation medium for production of ovalbumin was used as described in US2014/0342396 A1 while using 5.5 g/L (NH₄)₂SO₄, feeding glucose (90%) together with maltodextrines (10%) and titrating to control pH using 12.5% ammonium solution. 0.23 g/L antifoam BT03 (van Meeuwen, The Netherlands).

### Fermentation of A. Niger

The medium for the fermentation in deep-well plates (DWP) is described above. The inoculum and fermentation were carried out in the following way. 200 µl of a glycerol strain stock was put on a PDA plate, 3 ml PM was added and the plate was first shaken to spread out the mycelium, and then incubated for 3 days at 32° C. The whole PDA+PM medium plate was harvested by adding 5 ml of PM medium on the top of the grown fungal mycelium, which was scratched with a T-spatula to loosen the mycelium. The whole mycelium was collected in a sterile tray to make more homogenic inoculum. 0.5 ml of the homogenized mycelium was transferred to an DWP. The DWP was incubated for 6 days at 34° C. at 350 rpm in an incubator (Infors minitron, 2.5 cm stroke). The supernatant was used for analysis using SDS-PAGE.

The medium for the fermentation in shake flasks (KM 3.0) is described above. The inoculum and fermentation were carried out in the following way. 200 µl of a glycerol strain stock was put on a PDA plate, 3 ml PM was added and the plate was first shaken to spread out the mycelium, and then incubated for 3 days at 32° C. The whole PDA+PM medium plate was harvested, which was scratched with a T-spatula to loosen the mycelium and the mycelium was inoculated in 300 ml baffled shake flasks with steristoppers with 35 ml GYE medium, and then incubated for 2 days at 32° C., 220 rpm in an incubator (Infors multritron, 2.5 cm stroke). 1 ml of the GYE culture is inoculated in 100 ml non-baffled shake flasks with steristoppers with 22 ml KM3.0 medium, and then incubated for 6 days at 32° C., 120 rpm in an incubator (Infors multritron, 2.5 cm stroke). The supernatant was used for analysis using SDS-PAGE.

The medium in the 5 L fermenter was prepared as described above. Glucose was fed to control glucose > 10 g/L. Stirring was done to mix the fermenter and provide oxygen enough to produce optimal protein amounts. pH was set at 6.5 pH and the fermenter was kept at 32° C. pH was controlled between 5 and 7 pH and temperature between 31 to 33° C.

### Purification of A. Niger Transformants

The primary transformants were re-streaked on minimal medium with hygromycin or acetamide and the growing (positive) transformants were analysed by colony PCR for the presence of the ovalbumin gene and hygromycin or acetamidase resistance encoding gene. The positive transformants after the colony PCR were transferred to sporulation agar for sporulation. Single sporulating colonies were selected and streaked on minimal medium with hygromycin or acetamide. Genomic DNA was isolated of single colonies for confirmation of the presence of the ovalbumin gene and hygromycin resistance or acetamidase encoding gene.

### SDS-PAGE and Western Blot Analysis of A.Niger Transformants

SDS-PAGE was carried out after the small-scale fermentation. Sample preparation was 7.8 µl supernatant, 3 µl NuPAGE® LDS Sample Buffer (4x) and 1.2 µl NuPAGE® Reducing Agent (10x), which was denaturated for 10 min at 95° C. 10 µl sample was loaded in a Bolt™ 8% Bis-Tris Plus SDS-PAGE and 5 µl BenchMark™ Unstained Protein Ladder was added. 1 liter 1x NuPAGE® running buffer was fresh prepared with 20x NuPAGE® SDS running buffer MOPS, 200 ml 1x NuPAGE® running buffer with 0.5 ml NuPAGE® antioxidant was added in the inner chamber of the system, the rest of the 1x NuPAGE® running buffer was added in the outer chamber. The SDS-PAGE ran according to manufacturing protocol. Staining of the SDS-PAGE was carried out with SimplyBlue Safestain according to manufacturing protocol. All chemicals used in the SDS-PAGE protocol are from Invitrogen/Thermo Fisher Scientific.

The supernatant of 5 (d5) and 6 (d6) days-old cultures grown in a 96-deep well plate (the small-scale fermentation) was analysed on the SDS-PAGE (left panel in FIG. 4) and blotted on the nitrocellulose membrane (right panel in FIG. 4) and detected with the hen ovalbumin polyclonal antibodies (Rabbit / IgG Ovalbumin Polyclonal Antibody, CAT# ThermoFisher PA1196 and Goat Anti-Rabbi IgG HRP-conjugate CAT# Sigma AB1225).

### Selection of Ovalbumin Sequences From NCBI Database and Their in Silico Analysis

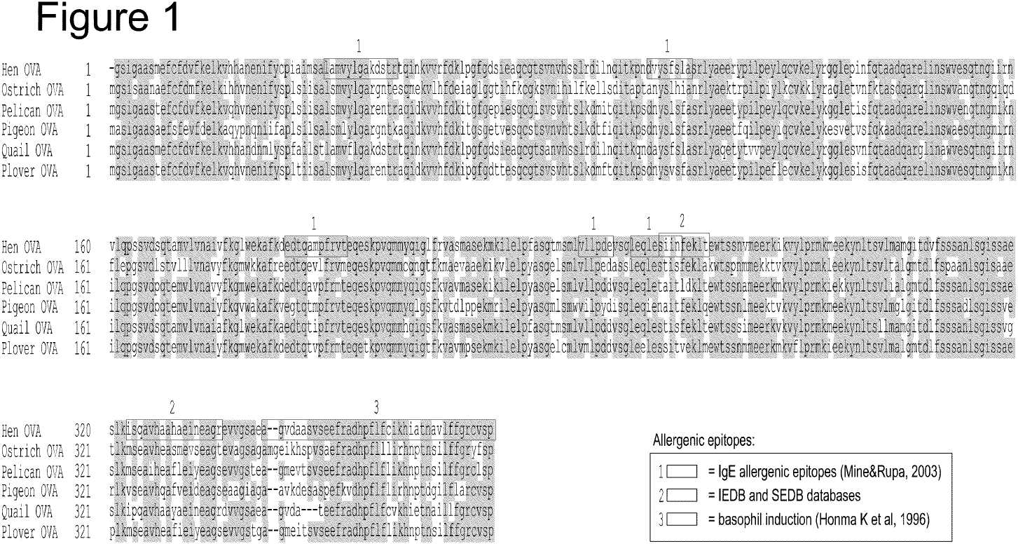

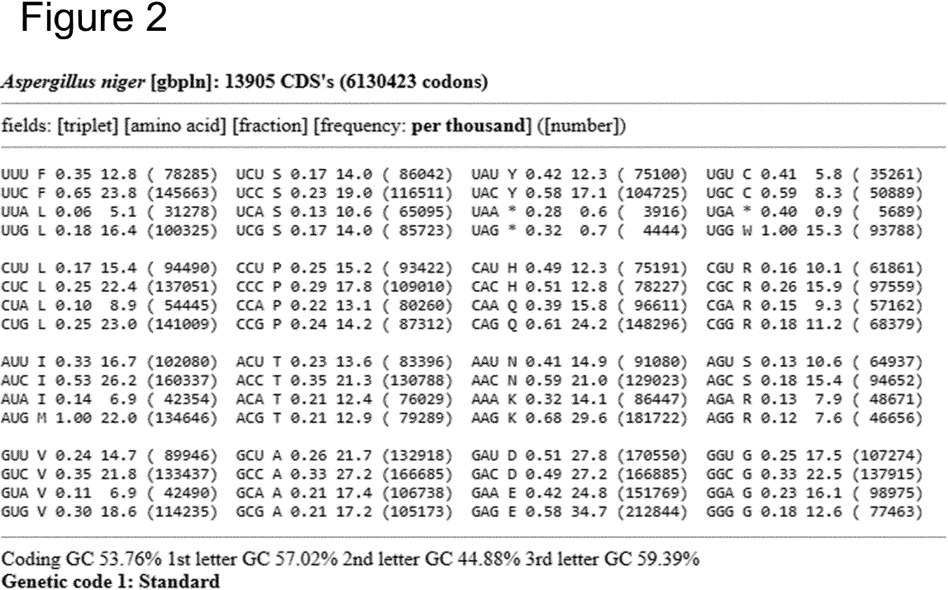

The hen ovalbumin protein sequence (GenBank accession number AAB59956.1) was used to Blast (Altschul., S. F., et al (1997) “Gapped BLAST and PSI-BLAST: a new generation of protein database search programs” Nucleic Acids Res. 25:3389-3402) the NCBI database in order to obtain alternative ovalbumin’s from different bird species. Multiple sequences were found, from which, a selection of five alternative hen ovalbumin was chosen according to the following criteria:
i) a history of human use of eggs of selected birds (information based on search in Wikipedia);
ii) the absence of hen egg allergenic epitopes (Mine Y. and Rupa P., (2003) Clin Exp Immunol 103, 446-453); allergen databases: IEDB - http:/www.iedb.org/home_v3.php and SEDB -http://sedb.bicpu.edu.in/home.php); and
iii) phylogenetic distance of birds (Jarvis E.D. et al., (2014), Whole-genome analyses resolve early branches in the tree of life of modern birds, Science 346 (6215), 1320-1331).
A selection of the ovalbumin sequences that fulfil these criteria, but are not limited to this selection, are from ostrich ( Struthio camelus australis)-, pelican ( Pelecanus crispus); pigeon ( Columba livia); quail ( Coturnix coturnix); and plover ( Charadrius vociferus) (see FIG. 1 and SEQ ID NOs. 2 - 6).

### Construction of Ovalbumin Expression Cassettes with Whole Genome Based Codon Usage

To overexpress selected ovalbumin in A. niger we constructed the expression cassettes in the following way:
i) the protein sequences (SEQ ID NOs. 1 to 6) were used as the input for an in-vitro synthesis of the corresponding DNA coding sequence. The codon optimization for expression in A. niger was based on the complete codon usage of A. niger CBS513.88 (See FIG. 2; https://www.kazusa.or.jp/codon/) and the gene synthesis was done by GenScript (US). The corresponding DNA coding sequences are mentioned in the sequence listing under the SEQ ID NOs. 7 to 12. At the beginning and the end of each sequence, Bsal enzyme restriction sites were added to facilitate the Golden Gate Reaction and cloned in pUC57-kan (SEQ ID NO.13).
ii) the glucoamylase (GenBank: AAP04499.1) secretory sequence (SEQ ID NO. 14) consisting of a pre-pro-peptide was used to drive the secretion of the ovalbumin extracellularly. The sequence was made synthetically and fused directly to the second amino acid (based on the native hen ovalbumin) of the corresponding ovalbumin amino acid sequence.
iii) in an alternative ovalbumin expression cassette, the ovalbumin coding sequence was fused to the truncated glucoamylase gene sequence coding for its first 502 amino acids. This glucoamylase sequence includes its secretory pre-pro sequence at the N-terminal end. Between the 502 amino acids of glucoamylase and the start of the mature ovalbumin sequence a synthetic peptide of 8 amino acids, which includes the KEX2 (Lys-Arg) cleavage site (SEQ ID NOs. 15 and 16) was inserted. A similar truncated glucoamylase protein sequence, that was used as a homologous carrier through the secretory pathway of A. niger, was reported to increase significantly production of a heterologous protein (Jeenes, D.J. et al., (1993), A truncated glucoamylase gene fusion for heterologous protein secretion from Aspergillus niger, FEMS Microbiology Letters 107, 267-272).

The regulatory sequences of the glucoamylase gene (GenBank: An03g06550) were used for driving a high expression of the gene of interest and ensuring an efficient transcript termination. The glaA promoter and the glaA terminator were PCR amplified from the host A. niger BZASNI.22a strain with primers 406 and 408 (SEQ ID NOs. 17 and 18) and primers 409 and 410 (SEQ ID NOs. 19 and 20), respectively, and purified with the Wizard® SV Gel and PCR clean-up system (Promega). These purified PCR products were ligated in the pCR™Blunt II-TOPO® vector (SEQ ID NO. 21) from the Zero Blunt™ TOPO™ PCR Cloning Kit (Thermo Fisher Scientific) and transformed to NEB 10-beta Competent E. coli cells. The plasmid from the positive E. coli transformants was checked by a restriction enzyme analysis and by DNA sequencing for the correct sequence and orientation of the inserts. See Table 1 for all constructed plasmids.

The expression cassette consisting of the 1000 bp glaA promoter, the pre-pro-peptide or the carrier protein, the ovalbumin coding sequence and the 600 bp glaA terminator were assembled using the NEB Golden Gate Assembly Kit (New England Biolabs, with plasmid pGGA, SEQ ID NO. 22) and transformed to NEB 10-beta Competent E. coli cells. The plasmid from the positive E. coli transformants was checked by a restriction enzyme analysis and by DNA sequencing for the correct sequence and orientation of the inserts. See Table 1 for all constructed plasmids. The construction of GLA protein fusions was carried out as mentioned above.

### Construction of Ovalbumin Expression Cassettes With Gene Specific Codon Usage

In order to test another approach for a codon usage optimization for the expression of heterologous proteins in A. niger we selected the highly expressed A. niger gene glaA, and based the optimisation of the codon usage on its codon preference. We constructed the expression cassettes in the following way:

The cDNA of hen ovalbumin (Genbank accession number MF321659.1) was used as the input for the optimization of the codon usage for expression of ovalbumin in A. niger. The A. niger glucoamylase (glaA) cDNA (NCBI reference number XM_001 390493.2) codon usage was analysed using the on-line available analysis tool, the Sequence Manipulation Suite (http://www.bioinformatics.org/sms2/codon usage.html) (see Table 3A.). Subsequently, the hen ovalbumin cDNA was codon-optimized using the codon-usage table of the glaA cDNA of A.niger CBS513.88 (Table 3A.) as the input using an on-line available DNA optimizing tool OPTIMISER (http://genomes.urv.es/OPTIMISER/; Puigbo P. et al., 2007; Nucl Acids Res 35; W126-W131). The resulting hen ovalbumin cDNA was further manually corrected to make the closest match to the glaA cDNA codon usage table (see Table 3.B, SEQ ID NO.32), taking into the account an equal distribution of changes throughout the whole cDNA sequence. The gene synthesis was done by GenScript (US). The methionine of the ovalbumin protein sequence was replaced by the glucoamylase (GLA) pre-pro sequence of A. niger (SEQ ID NO.14) to direct the secretion of the protein extracellularly (Note that ovalbumin comprises an internal signal sequence (residues 21-47), which is not cleaved off, but remains as part of the mature protein). The corresponding DNA coding sequence is mentioned in the sequence listing under the SEQ ID NO.30. Genscript synthesized the hen ovalbumin coding sequence in the destination plasmid pUC57 (SEQ ID NO.31), which was named BZESCO.21 (SEQ ID NO.32).

Alternatively, the ovalbumin coding sequence was fused to the truncated glucoamylase gene sequence (SEQ ID NO. 33), which is fulfilling the role of a carrier through the A. niger secretory pathway. To release the secreted ovalbumin from the GLA carrier, the enterokinase cleavage site (DDDDK) was added between the carrier protein and the start of the native ovalbumin. The truncated glucoamylase gene, with the added enterokinase cleavage site, was PCR amplified with primers 276 and 286 (SEQ ID NOs. 34 and 35) from genomic DNA of A. niger BZASNI.22a.

Construction of both ovalbumin constructs (with and without the GLA carrier fusion) were made using fusion PCR. For the construct without the GLA carrier fusion, the promoter of glaA was PCR amplified with primers 276 and 277 (SEQ ID NOs. 34 and 36) and the terminator with primers 280 and 281 (SEQ ID NOs. 37 and 38), both from genomic DNA of A. niger BZASNI.22a. The hen ovalbumin ORF was PCR amplified with the primers 278 and 279 (SEQ ID NOs. 39 and 40) using the plasmid BZESCO.21 (SEQ ID NO. 32) as a template. For the GLA carrier - ovalbumin fusion construct, the promoter of glaA and the truncated glaA gene corresponding to the GLA carrier protein were PCR amplified with the primers 276 and 286 (SEQ ID NOs. 34 and 35) as well as the terminator (primers 280 and 281, corresponding to the SEQ ID NO. 37 and 38, respectively), both from the genomic DNA of A.niger BZASNI.22a. The hen ovalbumin ORF was PCR amplified with the primers 287 and 279 (SEQ ID NOs. 41 and 40) from BZESCO.21 (SEQ ID NO. 32). The expression cassette parts described above were assembled using an overlap extension PCR with primers aligning at the 5′ and 3′ ends of the cassette, respectively. The obtained products were cloned into pUC57-Brick vector (SEQ ID NO. 42) by restriction-ligation. The complete construct without the GLA carrier fusion was named BZESCO.22 (SEQ ID NO. 43) and the GLA carrier fusion construct was named BZESCO.23 (SEQ ID NO. 44).The hen ovalbumin expression cassettes (with and without the GLA carrier fusion) were PCR amplified with primers 276 and 281 (SEQ ID NOs. 34 and 38) and transformed with the DNA fragment containing the hygromycin selection marker PCR amplified from pCNS43 (SEQ ID NO. 45) with primers 404 and 405 (SEQ ID NOs. 46 and 47) to the A. niger strain BZASNI.22a.

To overexpress the other selected ovalbumin encoding genes based on the codon usage optimization of A. niger highly expressed gene glaA, we constructed the expression cassettes in the following way: i) the protein sequences of ostrich, pelican, pigeon, quail and plover ovalbumins (SEQ ID NOs. 2 to 6), were used as the input for gene synthesis by GenScript (US) using the codon-usage table of the glaA cDNA of A. niger CBS513.88 (Table 3A.) and their in house gene synthesis, codon optimization algorithm. The methionine at the position +1 of the ovalbumin protein sequences was replaced by the glucoamylase (GLA) pre-pro-peptide sequence of A. niger (SEQ ID NO.14) to direct the secretion of the protein extracellularly (Note that ovalbumin comprises an internal signal sequence (residues 21-47), which is not cleaved off, but remains as part of the mature protein). The corresponding DNA coding sequences are mentioned in the sequence listing under the SEQ ID NOs. 65 to 69. The sequences were delivered by GenScript in pUC57-kan (SEQ ID NO.13) and were further modified at the 5′ and the 3′ end by adding the Bsal enzyme restriction sites to facilitate the Golden Gate Reaction. The glaA promoter and the glaA terminator were PCR amplified from the host A. niger BZASNI.22a strain with primers 406 and 408 (SEQ ID NOs. 17 and 18) and primers 409 and 410 (SEQ ID NOs.19 and 20), respectively, and purified with the Wizard® SV Gel and PCR clean-up system (Promega). These purified PCR products were ligated in the pCR™Blunt II-TOPO® vector (SEQ ID NO. 21) from the Zero Blunt™ TOPO™ PCR Cloning Kit (Thermo Fisher Scientific) and transformed to NEB 10-beta Competent E. coli cells. The plasmid from the positive E. coli transformants was checked by a restriction enzyme analysis and by DNA sequencing for the correct sequence and orientation of the inserts. See Table 1 for all constructed plasmids.

The expression cassette consisting of the 1000 bp glaA promoter, the glucoamylase pre-pro-peptide with the ovalbumin coding sequence and the 600 bp glaA terminator were assembled using the NEB Golden Gate Assembly Kit (New England Biolabs, with plasmid pGGA, SEQ ID NO.22) and transformed to NEB 10-beta Competent E. coli cells. The plasmid from the positive E. coli transformants was checked by a restriction enzyme analysis and by DNA sequencing for the correct sequence and orientation of the inserts. See Table 1 for all constructed plasmids (SEQ ID NO.70 to 74).

For the A. niger genome targeted single cross-over integration construct, a 1500 bp region downstream of the glaA locus was used (marked as 3″ glaA). This 3″ glaA part was PCR amplified from the host A. niger BZASNI.22a with primers 728 and 729 (SEQ ID NO. 75 and 76). The different expression cassettes (glaA promoter — codon optimized ovalbumin gene — glaA terminator) were PCR amplified from Golden Gate constructs (SEQ ID NO. 70 to 74) and BZESCO.22 (SEQ ID NO.43) with primers 733 and 734 (SEQ ID NO. 77 and 78). The plasmid backbone part was PCR amplified from plasmid pUC19 (SEQ ID NO. 79) with primers 431 and 432 (SEQ ID NO. 80 and 81). All PCR products were purified with the Wizard® SV Gel and PCR clean-up system (Promega). The integration construct consisting of the 1500 bp 3″ glaA part, the different expression cassettes and the plasmid backbone were assembled using the NEB Gibson Assembly Cloning Kit (New England Biolabs) and transformed to NEB 5-alpha Competent E. coli cells. The plasmids from the positive E. coli transformants were checked by a restriction enzyme analysis and by DNA sequencing for the correct sequence and orientation of the inserts. See Table 1 for all constructed plasmids (SEQ ID NO.82 to 87).

Table 3. Codon usage table (frequency of the codon usage for a certain amino acid, e.g. for instance: UUU - F - 0.18, means 18% of all F are coded by UUU) of the glucoamylase (glaA) cDNA, from Aspergillus niger (A) and the glaA codon-based optimized hen ovalbumin cDNA (B).

A.
codon usage table of gla A from A.niger CBS513.88

| | | | | | | | | | | | |
|---|---|---|---|---|---|---|---|---|---|---|---|
| UUU | F | 0.18 | UCU | S | 0.18 | UAU | Y | 0.22 | UGU | C | 0.3 |
| UUC | F | 0.82 | UCC | S | 0.22 | UAC | Y | 0.78 | UGC | C | 0.7 |
| UUA | L | 0 | UCA | S | 0.05 | UAA | * | 0 | UGA | * | 0 |
| UUG | L | 0.13 | UCG | S | 0.16 | UAG | * | 1 | UGG | W | 1 |
| CUU | L | 0.06 | CCU | P | 0.18 | CAU | H | 0 | CGU | R | 0.2 |
| CUC | L | 0.35 | CCC | P | 0.45 | CAC | H | 1 | CGC | R | 0.35 |
| CUA | L | 0.04 | CCA | P | 0 | CAA | Q | 0.18 | CGA | R | 0.2 |
| CUG | L | 0.42 | CCG | P | 0.36 | CAG | Q | 0.82 | CGG | R | 0.15 |
| AUU | I | 0.5 | ACU | T | 0.27 | AAU | N | 0.24 | AGU | S | 0.14 |
| AUC | I | 0.46 | ACC | T | 0.53 | AAC | N | 0.76 | AGC | S | 0.26 |
| AUA | I | 0.04 | ACA | T | 0.07 | AAA | K | 0 | AGA | R | 0.05 |
| AUG | M | 1 | ACG | T | 0.14 | AAG | K | 1 | AGG | R | 0.05 |
| GUU | V | 0.14 | GCU | A | 0.38 | GAU | D | 0.48 | GGU | G | 0.3 |
| GUC | V | 0.36 | GCC | A | 0.29 | GAC | D | 0.52 | GGC | G | 0.47 |
| GUA | V | 0.05 | GCA | A | 0.15 | GAA | E | 0.35 | GGA | G | 0.15 |
| GUG | V | 0.45 | GCG | A | 0.17 | GAG | E | 0.65 | GGG | G | 0.09 |

B.
codon usage table hen OVA optimized for expression in A.niger based on the gla A A.niger sequence

| | | | | | | | | | | | |
|---|---|---|---|---|---|---|---|---|---|---|---|
| UUU | F | 0.2 | UCU | S | 0.18 | UAU | Y | 0.2 | UGU | C | 0.33 |
| UUC | F | 0.8 | UCC | S | 0.21 | UAC | Y | 0.8 | UGC | C | 0.67 |
| UUA | L | 0 | UCA | S | 0.05 | UAA | * | 0 | UGA | * | 0 |
| UUG | L(s) | 0.13 | UCG | S | 0.16 | UAG | * | 1 | UGG | W | 1 |
| CUU | L | 0.06 | CCU | P | 0.21 | CAU | H | 0 | CGU | R | 0.2 |
| CUC | L | 0.34 | CCC | P | 0.43 | CAC | H | 1 | CGC | R | 0.33 |
| CUA | L | 0.06 | CCA | P | 0 | CAA | Q | 0.2 | CGA | R | 0.2 |
| CUG | L(s) | 0.41 | CCG | P | 0.36 | CAG | Q | 0.8 | CGG | R | 0.13 |
| AUU | I | 0.48 | ACU | T | 0.27 | AAU | N | 0.24 | AGU | S | 0.13 |
| AUC | I | 0.48 | ACC | T | 0.53 | AAC | N | 0.76 | AGC | S | 0.26 |
| AUA | I | 0.04 | ACA | T | 0.07 | AAA | K | 0 | AGA | R | 0.07 |
| AUG | M(s) | 1 | ACG | T | 0.13 | AAG | K | 1 | AGG | R | 0.07 |
| GUU | V | 0.16 | GCU | A | 0.4 | GAU | D | 0.5 | GGU | G | 0.26 |
| GUC | V | 0.35 | GCC | A | 0.29 | GAC | D | 0.5 | GGC | G | 0.47 |
| GUA | V | 0.03 | GCA | A | 0.14 | GAA | E | 0.36 | GGA | G | 0.16 |
| GUG | V | 0.45 | GCG | A | 0.17 | GAG | E | 0.64 | GGG | G | 0.11 |

### Construction of Alternative Gla Carrier Fusion Ovalbumin Expression Cassettes With Gene Specific Codon Usage

To overexpress selected ovalbumin in A. niger we constructed an alternative ovalbumin expression cassette with different size of truncated glucoamylase from A. niger. The selection of the truncated carrier sequence was based on the tertiary structure of glucoamylase, the pl and the Mw of the truncated protein was taken into account to facilitate the downstream processing of expressed ovalbumin. The optimized ovalbumin coding sequence was fused to 3 different truncated glucoamylase gene sequences, coding for its first 54, 100 and 502 amino acids of GLA, respectively. All these glucoamylase sequences included its own secretory pre-pro-peptide sequence at the N-terminal end.

The glucoamylase sequence, which consist of the first 54 amino acids (GLA54, SEQ ID NOs 88 and 89) translates to the pre-pro-peptide sequence, the first alpha helix of the glucoamylase protein and ends with the addition of KEX2 (Lys-Arg) cleavage site. The glucoamylase sequence, which consist of the first 100 amino acids (GLA100, SEQ ID NOs 90 and 91) translates to the pre-pro-peptide sequence and the first 3 alpha helixes of the glucoamylase protein and ends with the addition of KEX2 (Lys-Arg) cleavage site. The glucoamylase sequence, which consist of the first 502 amino acids (SEQ ID NOs. 130 and 16) translates to the pre-pro-peptide sequence and the glucoamylase protein without the starch binding domain. Between the 502 amino acids of glucoamylase and the start of the mature ovalbumin sequence, a synthetic peptide of 8 amino acids, which includes the KEX2 (Lys-Arg) cleavage site, was inserted. The corresponding DNA coding sequences of the 3 different truncated glucoamylase variants were synthesized by GenScript (US) in the destination plasmid pUC57-kan (SEQ ID NO.13). At the 5′ and the 3′ end of each sequence Bsal enzyme restriction sites were added to facilitate the Golden Gate Reaction.

A similar truncated glucoamylase protein sequence, that was used as a homologous carrier through the secretory pathway of A. niger, was reported to increase significantly production of a heterologous protein (Jeenes, D.J. et al., 1993 supra). The regulatory sequences of the glucoamylase gene (GenBank: An03g06550) were used for driving a high expression of the gene of interest and ensuring an efficient transcript termination.

For constructing of the expression cassettes, the glaA promoter and the glaA terminator were PCR amplified from the host A. niger BZASNI.22a strain with primers 406 and 408 (SEQ ID NOs. 17 and 18) and primers 409 and 410 (SEQ ID NOs.19 and 20), respectively, and purified with the Wizard® SV Gel and PCR clean-up system (Promega). These purified PCR products were ligated in the pCR™Blunt ll-TOPO® vector (SEQ ID NO. 21) from the Zero Blunt™ TOPO™ PCR Cloning Kit (Thermo Fisher Scientific) and transformed to NEB 10-beta Competent E. coli cells. The plasmid from the positive E. coli transformants was checked by a restriction enzyme analysis and by DNA sequencing for the correct sequence and orientation of the inserts. See Table 1 for all constructed plasmids.

The ovalbumin coding sequences were PCR amplified with primers 715 and 422 for OVA ostrich (SEQ ID NO.92 and 93) from the GenScript (US) synthesized DNA coding sequence with SEQ ID NO.65, primers 716 and 424 for OVA Plover (SEQ ID NO94 and 95) from the GenScript (US) synthesized DNA coding sequence with SEQ ID NO.69, primers 717 and 426 for OVA Pelican (SEQ ID NO.96 and 97) from the GenScript (US) synthesized DNA coding sequence with SEQ ID NO.66, primers 718 and 728 for OVA Pigeon (SEQ ID NO.98 and 99) from the GenScript (US) synthesized DNA coding sequence with SEQ ID NO.67 and primers 719 and 430 for OVA Quail (SEQ ID NO.100 and 101) from the GenScript (US) synthesized DNA coding sequence with SEQ ID NO.68. All PCR amplified ovalbumin genes were purified with the Wizard® SV Gel and PCR clean-up system (Promega). The expression cassette consisting of the 1000 bp glaA promoter, the 3 different glucoamylase variants, codon optimized ovalbumin coding sequence and the 600 bp glaA terminator were assembled using the NEB Golden Gate Assembly Kit (New England Biolabs, with plasmid pGGA, SEQ ID NO.22) and transformed to NEB 10-beta Competent E. coli cells. The plasmid from the positive E. coli transformants was checked by a restriction enzyme analysis and by DNA sequencing for the correct sequence and orientation of the inserts. See Table 1 for all constructed plasmids (SEQ ID NO.102 to 116).

For the construction of the GLA carrier fusion constructs with the hen ovalbumin (SEQ ID NO.30), already constructed plasmids were used as PCR templates to PCR amplify: i) the glaA promoter with the 3 different glucoamylase variants and ii) to PCR amplify the codon optimized hen ovalbumin, glaA terminator, the plasmid backbone and the single cross-over integration part - 3″ glaA. In Table 4 is described which part of the GLA carrier expression cassette is PCR amplified from which template construct with a primer combination.

Table 4

Primer combinations for the GLA carrier fusion hen ovalbumin constructs

| PCR target | PCR template | Primer combination |
|---|---|---|
| glaA promoter-GLA54 | pBZ0050 (SEQ ID NO.106) | 823 and 824 (SEQ ID NO. 117 and 118) |
| glaA promoter-GLA100 | pBZ0055 (SEQ ID NO.111) | 823 and 825 (SEQ ID NO. 117 and 119) |
| glaA promoter-GLA502 | pBZ0060 (SEQ ID NO. 116) | 823 and 826 (SEQ ID NO. 117 and 120) |
| OVA-glaA terminator | pBZ0061 (SEQ ID NO.82) | 827 and 830 (SEQ ID NO. 121 and |
| backbone-3″ glaA (for GLA54 fusion) | | 124) |
| OVA-glaA terminator | pBZ0061 (SEQ ID NO.82) | 828 and 830 (SEQ ID NO. 122 and |
| backbone-3″ glaA (for GLA100 fusion) | | 124) |
| OVA-glaA terminator | pBZ0061 (SEQ ID NO.82) | 829 and 830 (SEQ ID NO. 123 and |
| backbone-3″ glaA (for GLA100 fusion) | | 124) |

All PCR products were purified with the Wizard® SV Gel and PCR clean-up system (Promega), assembled using the NEB Gibson Assembly Cloning Kit (New England Biolabs) and transformed to NEB 5-alpha Competent E. coli cells. The plasmids from the positive E. coli transformants were checked by a restriction enzyme analysis and by DNA sequencing for the correct sequence and orientation of the inserts. See Table 1 for all constructed plasmids (SEQ ID NO. 125 to 127).

### Transformation of Overexpression Cassettes and Selection of A. Niger Transformants

The transformation of A. niger was based on the protocol of Balance et al. (Ballance D.J. et al., (1983), Transformation of Aspergillus nidulans by the orotidine-5′-phosphate decarboxylase gene of Neurospora crassa. Biochem. Biophys. Res. Comm. 112,284-289). For the transformation, the entire expression cassette, containing the ovalbumin-encoding gene and the glaA regulatory sequences, and the various GLA based carrier sequences, was PCR amplified using the primers aligning at the 5′ glaA promoter (forward primer 406, SEQ ID NO. 17) and the 3′gla A terminator (reverse primer 410, SEQ ID NO. 20) DNA sequence. This DNA fragment was co-transformed with the hygromycin selection marker, which was PCR amplified from pCNS43 (see Table 1) with primers 404 and 405 (SEQ ID NOs. 46 and 47) and the transformants were selected on plates containing minimal medium supplemented with the antibiotic hygromycin. The primary transformants were re-streaked on minimal medium with hygromycin and the growing (positive) transformants were analysed by colony PCR for the presence of the ovalbumin gene and hygromycin (See Table 5 for the primer combinations, SEQ ID NOs. 48 to 61). The positive colonies were purified via a single-spore purification procedure and used to prepare an inoculum for a small-scale fermentation.

The transformation can also be carried out with a DNA fragment encoding the A.nidulans acetamidase gene as the selection marker. The corresponding DNA fragment can be PCR amplified from pBZ0026 (SEQ ID NO. 62) with primers 411 and 415 (SEQ ID NOs. 63 and 64). The transformants were selected on plates containing minimal medium supplemented with acetamide as the sole nitrogen source. The single cross-over integration constructs with codon optimization of ovalbumin based on the glaA gene and the different GLA fusion constructs with hen ovalbumin were transformed with the acetamide selection marker and were PCR amplified for transformation with primers 787 and 789 (SEQ ID NO.128 & 129). The primary transformants were analysed by colony PCR for the presence of the ovalbumin-encoding gene and the further analysis proceeded as described above for the hygromycin-ovalbumin co-transformants.

Table 5

Primer combinations diagnostic PCRs

| Target | Forward primer | Reverse primer |
|---|---|---|
| OVA ostrich | 553 | 554 |
| OVA plover | 555 | 556 |
| OVA pelican | 557 | 558 |
| OVA pigeon | 559 | 560 |
| OVA quail | 561 | 562 |
| hygromycin | 336 | 337 |
| amdS | 400 | 401 |

### Small Scale Fermentation in Deep-Well Plates

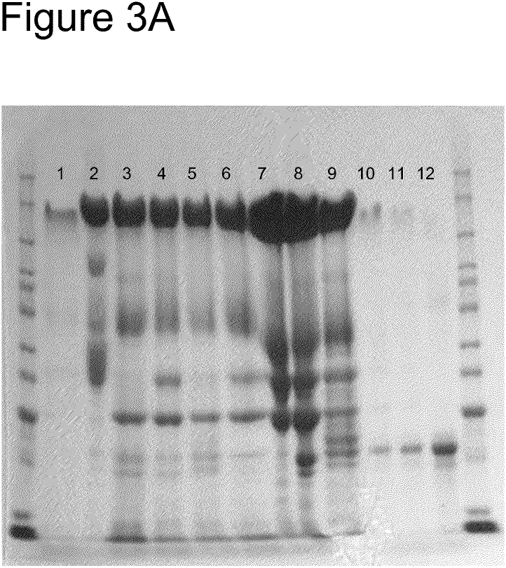

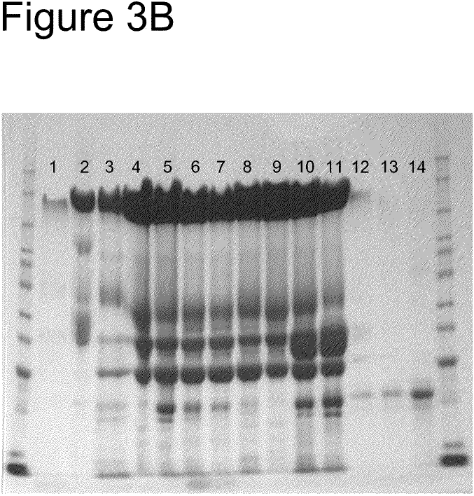

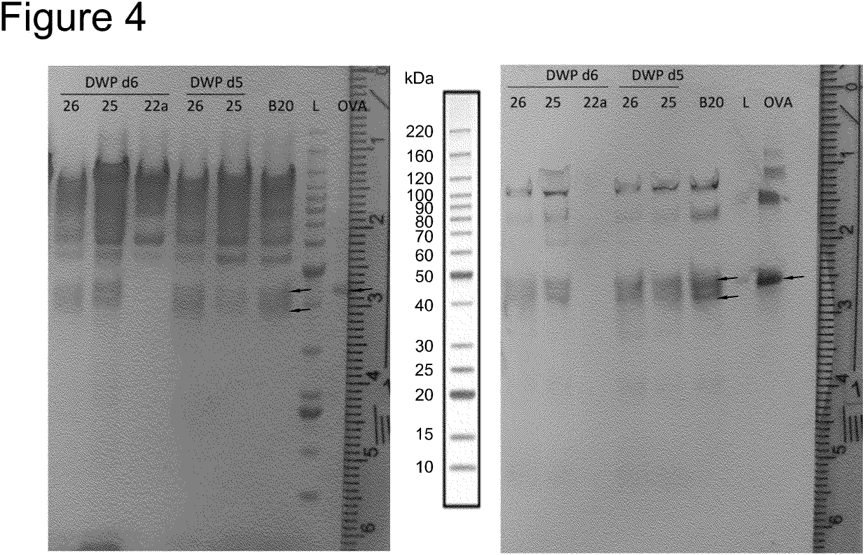

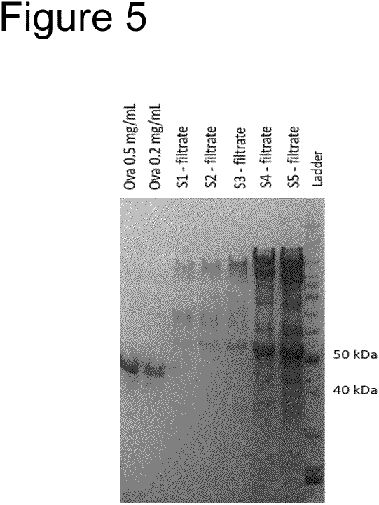

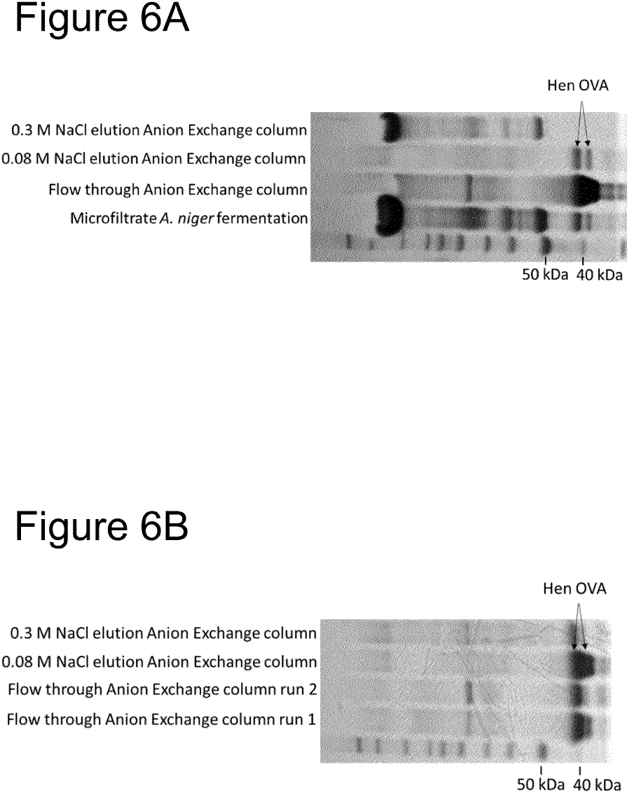

The fermentation was carried out up to the glucose consumption (in total about 6 days) and the supernatant of the cultures was harvested in different days of the fermentation. It was analysed on SDS-PAGE (see FIGS. 3A and 3B). To confirm the identity of the expressed hen ovalbumin, the SDS-PAGE blots were transferred to a nitrocellulose membrane and detected by Western blot using the commercially available hen ovalbumin polyclonal antibodies (see FIG. 4). From this blot (and also based on a comparison with the mobility of the commercial hen ovalbumin) it was assumed that the SDS-PAGE protein bands showing a similar mobility on the SDS-PAGE gel as the hen ovalbumin are representing the overexpressed ovalbumin of the selected birds. For the most promising transformants a large scale (5 L) fermentation was performed.

Based on the SDS-PAGE analysis of the various ovalbumin overexpressing transformants, the highest expression was obtained for the glaA codon optimized hen ovalbumin compared to the whole A. niger genome based codon optimisation of hen or other ovalbumin. Furthermore, the overexpressed hen ovalbumin appeared as two different Mw protein bands (FIG. 4) when fermented on the small scale similarly to the ovalbumin from pelican. On the other hand, the quail ovalbumin’s appeared as a single band migrating at the size of the commercial ovalbumin (FIG. 3A). Expression of ostrich and pigeon ovalbumin’s was verified using the LC-MS/MS protein identification method provided by an external service lab (data not shown). All different GLA carrier fusion constructs have hen ovalbumin production based on SDS-PAGE analysis (FIG. 3B).

### Small Scale Fermentation in Shake Flasks

The fermentation was carried out up to the glucose consumption (in total about 6 days) and the supernatant of the cultures was harvested in different days of the fermentation. The rounds per minute (rpm) of the incubator (Infors Multritron, 2.5 cm stroke) was critical for the production of ovalbumin in shake flasks. With 120 rpm production was very good, however with 220 rpm no production of ovalbumin was seen. It was analysed on SDS-PAGE (see FIGS. 3A and 3B). Power input can be calculated according to methods as described by Wolf Klockner and Jochen Buchs in Trends in Biotechnology June 2012, Vol 30, No 6.

### Fermentation in 5 L Fermenter

One of the best hen ovalbumin producing transformants, A.niger BZASNI.33, was grown in a 5 L fermenter. FIG. 5 shows the results of the SDS-PAGE analysis of the microfiltrated supernatant. From 40 hrs onwards, the production of the hen ovalbumin could be detected. Compared to the hen ovalbumin produced in the deep-well plate or in shake flask (FIGS. 3 and 4), in the 5 L fermentation broth the hen ovalbumin appeared as a single band.

### Stirring Albumen and Albumen Broth

The properties of hen egg white (with 54% ovalbumin) was assessed in a stirred fermenter. Above 600 rpm in a Cplus fermenter of Sartorius at standard configuration no solid egg white was observed, but from 900 rpm and higher, solid egg white appeared. At 600 rpm the power input is 1.4 kW/m3. Egg solids appearing after 22 hrs stirring at 32° C. in Cplus fermenter Sartorius.

| | | | | | |
|---|---|---|---|---|---|
| Rpm | 300 | 600 | 900 | 1200 | 1500 |
| Egg solids appearing | - | - | + | ++ | +++ |

### Purification of the Ovalbumin

Anion exchange chromatography was used as an example for purification of ovalbumin. The buffer for binding and eluting the protein(s), such as potassium phosphate or Tris-HCI buffer, had a molarity ranging from 20 to 50 mM. The pH of the buffer varied from 6 to 9. The salt concentration (such as NaCl), at which the ovalbumin eluted, ranges between 0.0 and 0.3 M.

Alternatively, the size exclusion chromatography was used to separate proteins with a substantially different size. The molarity of the buffer (such as potassium phosphate or Tris-HCl buffer) ranges between 10 and 50 mM. The pH of the buffer ranges between 6 and 9. The molarity of the salt (such as NaCl) ranges between 0.1 and 0.3 M. The flow rate of the isocratic elution ranges from 10 to 200 cm/h.

As an example, a purification of overexpressed hen ovalbumin from A. niger (BZASNI.60) microfiltrate using Anion Exchange Chromatography is shown in FIG. 6A. Ovalbumin was purified using the following protocol. The microfiltrate was diafiltrated against the starting eluent, 25 mM potassium phosphate buffer, pH 8. The sample was loaded on the Anion Exchange Column (HiTrap Capto Q AEC column, 5 mL, Cytiva). The flow-through of the loaded sample was collected. Next, the protein was eluted at 0.08 M NaCl in 25 mM potassium phosphate buffer, pH 8. All other proteins were eluted at 0.3 M NaCl in 25 mM potassium phosphate buffer, pH 8. The protein eluted at 0.08 M NaCl contains ovalbumin at approximately 90-98% purity. The flow-through contains ovalbumin at approximately 70-90% purity. To further purify the ovalbumin, this sample can be used as start sample for the second AEC purification. The second AEC purification is identical to the first one, except for the start sample, which is the flow through of the first AEC purification. An example of this is shown in FIG. 6B. The recovery of this purification was 85%.

### Testing Product

### A) Foaming Capacity

A protein content ranging from 10 to 150 mg/mL is used. The pH of the testing solutions typically ranges from 2 to 11. The agitation time ranges from 1 to 20 min using a suitable method (e.g. homogenization, blending), after which the foam is transferred to a suitable measuring cylinder. The total foam volume after 30 s is observed using a measuring cylinder and defines the foam capacity. The total foam volume is observed over a time ranging from 1 to 24 h and the rate at which the foam volume decreases defines the foam stability.

### B) Solubility

A protein content ranging from 1 to 10 g is added to an amount ranging from 10 to 1000 mL of buffer solution. The pH of the buffer solution ranges from 2 to 12, more preferably from 3 to 7. The protein solution is stirred for a time ranging from 30 to 120 minutes. Hereafter, the solution is centrifuged for a time of 10 to 60 minutes at a speed of 2000 to 20000 xg. Hereafter, the protein content of the supernatant is measured.

### C) Emulsification

A protein content ranging from 1 to 100 mg/mL, is suspended in a solution with a pH ranging from 2 to 10. Hereafter the solution is mixed with a suitable oil. The wt% of the protein solution in the final mixture ranges from 50 wt% to 100 wt%. The wt% of the oil in the final mixture ranges from 0 wt% to 50 wt%. The mixture is agitated for a time ranging from 1 to 10 min. The total volume of the emulsified phase, the water phase and the oil phase are observed using a measuring cylinder. The ratio between the phases defines the emulsion ability. The total volume of the emulsified phase, the water phase and the oil phase are recorded for a time ranging from 1 to 100 h. The rate at which the volumes change defines the emulsion stability.

Alternatively, the emulsifying properties of the protein of interest is based on the turbidity of the emulsion. After the emulsion is prepared, as described, a sample is taken and diluted with a suitable solution (e.g. sodium dodecyl sulfate) in a range of 10-500 times dilution. The turbidity of the dilution is measured at a suitable absorbance (e.g. for sodium dodecyl sulfate: 500 nm). The emulsifying activity index is then calculated using the absorbance, light path of the spectrophotometer, volume of the oil phase and concentration of protein before the emulsion is formed. The emulsion stability can be determined similarly by taking emulsion samples at time points in a range of 0-24 h, after which the turbidity is measured as described above. The emulsifying activity index over time indicates the emulsion stability. Next, the emulsion is heated for a time range of 10 to 60 min at a temperature range of 60 to 100° C. After the emulsion has cooled, a sample is taken, and its turbidity measured as described previously. After calculating the emulsifying activity index of these samples, the values can be used to calculate the emulsion stability.

### D) Gelling

A protein content ranging from 30 to 200 mg/mL is used. The salt (e.g. sodium chloride) molarity ranges between 0 and 200 mM. The pH of the solution ranges between 3 and 9. The solution is heated for a time range of 45 to 120 minutes at a temperature range of 60 to 100° C. Hereafter, the gel is cooled to room temperature and ready to undergo rheological measurements.

### Testing Product - Allergenicity

Sera of sensitized or allergic people containing IgE antibodies are used to perform in vitro allergenicity studies and to examine the allergenicity of a protein.

ELISA (Enzyme Linked Immunosorbent Assay) and immunoblotting are used to investigate the capability of a protein to bind allergen specific IgE antibodies. IgE antibodies from people allergic to Gal d 2 (the main hen ovalbumin allergen) are also used in immunoblotting assays to characterize Gal d 2 epitopes. The capability of a protein to bind IgE antibodies does not always mean that the binding will cause the release of inflammatory mediators and, therefore, an allergic reaction.

The capability of a protein to induce the release of inflammatory mediators is usually explored through the Basophilic Activation Test (BAT). In BAT basophil cells from allergic individuals are exposed to the allergen and the production of histamine upon allergen exposure is observed.

### REFERENCES

1. Duong-Ly, K. C. & Gabelli, S. B. Salting out of proteins using ammonium sulfate precipitation. in Methods in enzymology 541, 85-94 (Elsevier, 2014).
2. Abeyrathne, E., Lee, H. Y. & Ahn, D. U. Sequential separation of lysozyme, ovomucin, ovotransferrin, and ovalbumin from egg white. Poult. Sci. 93, 1001-1009 (2014).
3. Asenjo, J. A. & Andrews, B. A. Aqueous two-phase systems for protein separation: a perspective. J. Chromatogr. A 1218, 8826-8835 (2011).
4. Rito-Palomares, M. Practical application of aqueous two-phase partition to process development for the recovery of biological products. J. Chromatogr. B 807, 3-11 (2004).
5. Pereira, M. M. et al. Single-step purification of ovalbumin from egg white using aqueous biphasic systems. Process Biochem. 51, 781-791 (2016).
6. Wen, C., Ge, T., Zhao, Y., Zhou, H. & Shi, L. Method for co-preparing ovalbumin and ovotransferrin. 7 (2019).
7. Meihu, M., Fang, G., Qun, H. & Xiaowei, Z. Method for jointly extracting a variety of proteins from egg white. 11 (2013).
8. Haq, A., Lobo, P. I., Al-Tufail, M., Rama, N. R. & Al-Sedairy, S. T. Immunomodulatory effect of Nigella sativa proteins fractionated by ion exchange chromatography. Int. J. Immunopharmacol. 21, 283-295 (1999).
9. Medve, J., Lee, D. & Tjerneld, F. Ion-exchange chromatographic purification and quantitative analysis of Trichoderma reesei cellulases cellobiohydrolase I, II and endoglucanase II by fast protein liquid chromatography. J. Chromatogr. A 808, 153-165 (1998).
10. Kopaciewicz, W., Rounds, M. A., Fausnaugh, J. & Regnier, F. E. Retention model for high-performance ion-exchange chromatography. J. Chromatogr. A 266, 3-21 (1983).
11. Rossomando, E. F. [24] Ion-exchange chromatography, in Methods in enzymology 182, 309-317 (Elsevier, 1990).
12. Gooding, K. M. & Schmuck, M. N. Comparison of weak strong high-performance anion-exchange chromatography. J. Chromatogr. A 327, 139-146 (1985).
13. Awade, A. C., Moreau, S., Molle, D., Brule, G. & Maubois, J.-L. Two-step chromatographic procedure for the purification of hen egg white ovomucin, lysozyme, ovotransferrin and ovalbumin and characterization of purified proteins. J. Chromatogr. A 677, 279-288 (1994).
14. Guerin-Dubiard, C. et al. Hen egg white fractionation by ion-exchange chromatography. J. Chromatogr. A 1090, 58-67 (2005).
15. Croguennec, T., Nau, F., Pezennec, S. & Brule, G. Simple rapid procedure for preparation of large quantities of ovalbumin. J. Agric. Food Chem. 48, 4883-4889 (2000).
16. Geng, F. et al. Co-purification of chicken egg white proteins using polyethylene glycol precipitation and anion-exchange chromatography. Sep. Purif. Technol. 96, 75-80 (2012).
17. Soni, B., Trivedi, U. & Madamwar, D. A novel method of single step hydrophobic interaction chromatography for the purification of phycocyanin from Phormidium fragile and its characterization for antioxidant property. Bioresour. Technol. 99, 188-194 (2008).
18. Shaltiel, S. Hydrophobic chromatography, in Methods in enzymology 34, 126-140 (Elsevier, 1974).
19. McCue, J. T. Theory and use of hydrophobic interaction chromatography in protein purification applications, in Methods in enzymology 463, 405-414 (Elsevier, 2009).
20. Queiroz, J. A., Tomaz, C. T. & Cabral, J. M. S. Hydrophobic interaction chromatography of proteins. J. Biotechnol. 87, 143-159 (2001).
21. Gooding, D. L., Schmuck, M. N., Nowlan, M. P. & Gooding, K. M. Optimization of preparative hydrophobic interaction chromatographic purification methods. J. Chromatogr. A 359, 331-337 (1986).
22. Watanabe, E., Tsoka, S. & Asenjo, J. A. Selection of chromatographic protein purification operations based on physicochemical properties. Ann. N. Y. Acad. Sci. 721, 348-364 (1994).
23. Narhi, L. O., Kita, Y. & Arakawa, T. Hydrophobic interaction chromatography in alkaline pH. Anal. Biochem. 182, 266-270 (1989).
24. Mori, S. & Barth, H. G. Size exclusion chromatography. (Springer Science & Business Media, 2013).
25. Andre, A. S. A. H.-K. & Schwarm, K. S. Size-Exclusion Chromatography for Preparative Purification of Biomolecules. (2016).
26. Luo, J., Zhou, W., Su, Z., Ma, G. & Gu, T. Comparison of fully-porous beads and cored beads in size exclusion chromatography for protein purification. Chem. Eng. Sci. 102, 99-105 (2013).
27. Kato, A., Ibrahim, H. R., Watanabe, H., Honma, K. & Kobayashi, K. Structural and gelling properties of dry-heated egg white proteins. J. Agric. Food Chem. 38, 32-37 (1990).
28. Hua, Y., Cui, S. W., Wang, Q., Mine, Y. & Poysa, V. Heat induced gelling properties of soy protein isolates prepared from different defatted soybean flours. Food Res. Int. 38, 377-385 (2005).
29. Rawdkuen, S., Sai-Ut, S., Khamsorn, S., Chaijan, M. & Benjakul, S. Biochemical and gelling properties of tilapia surimi and protein recovered using an acid-alkaline process. Food Chem. 112, 112-119 (2009).
30. Egelandsdal, B. Heat-induced gelling in solutions of ovalbumin. J. Food Sci. 45, 570-574 (1980).
31. Shitamori, S., Kojima, E. & Nakamura, R. Changes in the heat-induced gelling properties of ovalbumin during its conversion to S-ovalbumin. Agric. Biol. Chem. 48, 1539-1544 (1984).
32. Matsudomi, N., Ishimura, Y. & Kato, A. Improvement of gelling properties of ovalbumin by heating in dry state. Agric. Biol. Chem. 55, 879-881 (1991).
33. Hatta, H., Kitabatake, N. & Doi, E. Turbidity and hardness of a heat-induced gel of hen egg ovalbumin. Agric. Biol. Chem. 50, 2083-2089 (1986).
34. Shigeru, H. & Shuryo, N. Contribution of hydrophobicity, net charge and sulfhydryl groups to thermal properties of ovalbumin. Can. Inst. Food Sci. Technol. J. 18, 290-295 (1985).
35. Creusot, N., Wierenga, P. A., Laus, M. C., Giuseppin, M. L. F. & Gruppen, H. Rheological properties of patatin gels compared with β-lactoglobulin, ovalbumin, and glycinin. J. Sci. Food Agric. 91, 253-261 (2011).
36. Morr, C. V et al. A collaborative study to develop a standardized food protein solubility procedure. J. Food Sci. 50, 1715-1718 (1985).
37. Kato, A., Ibrahim, H. R., Watanabe, H., Honma, K. & Kobayashi, K. New approach to improve the gelling and surface functional properties of dried egg white by heating in dry state. J. Agric. Food Chem. 37, 433-437 (1989).
38. Jambrak, A. R., Mason, T. J., Lelas, V., Herceg, Z. & Herceg, I. L. Effect of ultrasound treatment on solubility and foaming properties of whey protein suspensions. J. Food Eng. 86, 281-287 (2008).
39. Bera, M. B. & Mukherjee, R. K. Solubility, emulsifying, and foaming properties of rice bran protein concentrates. J. Food Sci. 54, 142-145 (1989).
40. Sathe, S. K. & Salunkhe, D. K. Functional properties of the great northern bean (Phaseolus vulgaris L.) proteins: emulsion, foaming, viscosity, and gelation properties. J. Food Sci. 46, 71-81 (1981).
41. Kato, A., Tsutsui, N., Matsudomi, N., Kobayashi, K. & Nakai, S. Effects of partial denaturation on surface properties of ovalbumin and lysozyme. Agric. Biol. Chem. 45, 2755-2760 (1981).
42. Pearce, K. N. & Kinsella, J. E. Emulsifying properties of proteins: evaluation of a turbidimetric technique. J. Agric. Food Chem. 26, 716-723 (1978).
43. Chobert, J. M., Sitohy, M. Z. & Whitaker, J. R. Solubility and emulsifying properties of caseins modified enzymatically by Staphylococcus aureus V8 protease. J. Agric. Food Chem. 36, 220-224 (1988).
44. Mine, Y., Noutomi, T. & Haga, N. Emulsifying and Structural Properties of Ovalbumin. J. Agric. Food Chem. 39, 443-446 (1991).
45. Yasumatsu, K. et al. Whipping and emulsifying properties of soybean products. Agric. Biol. Chem. 36, 719-727 (1972).
46. Kato, A., Murata, K. & Kobayashi, K. Preparation and characterization of ovalbumin-dextran conjugate having excellent emulsifying properties. J. Agric. Food Chem. 36, 421-425 (1988).
47. Owen ward, Production of recombinant proteins by filamentous fungiBiotechnology Advances 30 (2012) 1119-1139
48. Marc. W.T. Werten et al. Production of protein-based polymers in Pichia pastoris, Biotechnology Advances 37 (2019) 642-666.
49. Klaas van t Riet & Johannes Tramper, Basic Bioreactor Design, Marcel Dekker Inc. ISBN 0-8247-8446-4 1991.
50. Albert. J.J. van Ooyen et al. Heterologous protein production in the yeast Kluyveromyces lactis, FEMS Yeast Res 6 (2006) 381-392.
51. Nisbet et al. (1981), The complete Amino-Acid sequence of hen ovalbumin; Eur.J.Biochem 115 (1981)
52. Yang et al. Cloning of a novel ovalbumin gene from quail oviduct and its heterologous expression in Pichia pastoris. Journal of Basic Microbiology (2009), 49, S73-S78.
53. Křížková L. et al. Expression of Japanese quail ovalbumin in Saccharomyces cerevisiae. Folia Microbiol (Praha). 1992;37(4):273-8
54. H. Visser et al. Development of a mature fungal technology and production platform for industrial enzymes based on a Myceliophthora thermophila isolate, previously known as Chrysosporium lucknowense C1 Industrial Biotechnology.Published in Volume: 7 Issue 3: Jul. 1, 2011
55. O. Mercereau-Puijalon et al. Synthesis of a chicken ovalbumin-like protein in the yeast Saccharomyces cerevisiae; Gene Volume 11, Issues 1-2, October 1980, Pages 163-167
56. Ito K. et al. Structural characteristics of hen egg ovalbumin expressed in yeast Pichia pastoris Biosci. Biotechnol. Biochem. 69 (4), 755-761 (2005).

1. A fungal host cell comprising an expression cassette comprising a nucleotide sequence coding for an animal-derived food-protein of interest, which nucleotide sequence is operably linked to at least one regulatory sequence that is capable of effecting expression of the encoded protein of interest by the fungal host cell.

2. The fungal host cell according to claim 1, wherein the regulatory sequence is the promoter of a highly expressed fungal protein.

3. The fungal host cell according to claim 1, wherein the nucleotide sequence coding for the animal-derived food-protein of interest is codon optimized with reference to the native codon usage of the highly expressed fungal protein from which the promoter is derived.

4. The fungal host cell according to claim 1, wherein the expression cassette is integrated in a locus of a gene coding for a highly expressed fungal protein.

5. The fungal host cell according to claim 1, wherein the expression cassette comprises a nucleotide sequence encoding a signal sequence from a highly expressed secreted fungal protein, and optionally a pro-sequence, operably linked in frame to the nucleotide sequence coding for the animal-derived food-protein of interest.

6. The fungal host cell according to claim 5, wherein the expression cassette encodes a fusion protein comprising, in a N- to C-terminal direction: an A. niger glucoamylase pre-pro sequence; optionally, 10, 20, 30, 40, 50, 60, 70, 80, 90 or 100% of the mature A. niger glucoamylase amino acid sequence; optionally, a cleavable linker polypeptide; fused to the N-terminus of the protein of interest.

7. The fungal host cell according to claim 1, wherein the host cell is a yeast or a filamentous fungus, preferably, a filamentous fungal host cell belong to a species selected from Alternaria alternata, Apophysomyces variabilis, Aspergillus spp., Aspergillus fumigatus, Aspergillus flavus, Aspergillus oryzae, Aspergillus niger, Aspergillus awamori, Aspergillus nidulans, Aspergillus terreus, Cladosphialophora spp., Fonsecaea pedrosoi, Fusarium spp., Fusarium oxysporum, Fusarium solani, Lichtheimia spp., Lichtheimia corymbifera, Lichtheimia ramosa, Myceliophthora spp., Myceliophthora thermophila, Rhizopus spp., Rhizopus microsporus, Rhizomucor spp., Rhizomucor pusillus, Rhizomucor miehei, Trichoderma spp., Trichoderma reesei, Trichophyton spp., Trichophyton interdigitale, and Trichophyton rubrum.

8. The fungal host cell according to claim 7, wherein the host cell is a strain of a filamentous fungus that has the ability to grow in a yeast-like morphology.

9. The fungal host cell according to claim 1, wherein animal-derived food-protein of interest is a hemeprotein, a milk protein or an egg protein.

10. The fungal host cell according to claim 9, wherein food-protein of interest is ovalbumin comprising an amino acid sequence with at least 50% identity to the amino acid sequence of an ovalbumin from a bird selected from the group consisting of chicken, pelican, quail, pigeon, ostrich, plover, turkey, duck, goose, gull, guineafowl, junglefowl, peafowl, partridge, pheasant, emu, rhea and kiwi.

11. A process for producing an animal-derived food-protein of interest, the process comprising the steps of:
a) culturing the fungal host cell as defined in claim 1 in a medium in a fermenter under condition conducive to the expression of the protein of interest; and,
b) optionally, recovery of the protein of interest.

12. The process according to claim 11, wherein in step a) the fungal host cell is cultured at a pH that is equal to or higher than pH 5.0, 5.5, 6.0, 6.5, 7.0 or 7.5, or that is equal to or lower than pH 9.0, 8.5 or 8.0.

13. The process according to claim 11, wherein in step a) the input of mechanical power into the medium in the fermenter is no more than 2.5, 2.0, 1.8, 1.6, 1.4, 1.0, 0.5, 0.2 or 0.1 kW/m3.

14. The process according to claim 11, wherein in step b) the recovery of the protein of interest comprises at least a first anion exchange step wherein the culture medium obtained in step a) is applied to an anion exchange column at an ionic strength that is less than the equivalent of 0.08 M NaCl at a pH in the range of pH 6 to 9 and wherein the protein of interest is collected in the flow-through, wherein, optionally the flow-through obtained in the first anion exchange step is subjected to a second anion exchange step under identical conditions as the first step and wherein the protein of interest is collected in the flow-through from the second anion exchange step.

15. A process for purifying an animal-derived food-protein of interest from a spent culture medium of a fungal host cell wherein the protein has been expressed, the process comprising the steps of:
a) a first anion exchange step wherein the spent culture medium is applied to an anion exchange column at an ionic strength that is less than the equivalent of 0.08 M NaCl at a pH in the range of pH 6 to 9 and wherein the protein of interest is collected in the flow-through, and,
b) optionally, a second anion exchange step wherein the flow-through obtained in step a) is subjected to anion exchange under identical conditions as the first step, and wherein the protein of interest is collected in the flow-through.

16. (canceled)

17. The fungal host cell according to claim 2, wherein the highly expressed fungal protein is selected from acid α-amylase, α-amylase, TAKA-amylase, glucoamylase, xylanase, cellobiohydrolase, pyruvate kinase, glyceraldehyde-phosphate dehydrogenase, alcohol dehydrogenase, aldehyde dehydrogenase, sucrase, acetamidase, superoxide dismutase.

18. The fungal host cell according to claim 17, the promoter is an A. niger glucoamylase promoter.

19. The fungal host cell according to claim 3, wherein the highly expressed fungal protein is an A. niger glucoamylase.

20. The fungal host cell according to claim 8, wherein the strain is Aspergillus niger strain CICC2462, or a strain that is a single colony isolate and/or a derivative of strain CICC2462.

21. The process according to claim 11, wherein the food-protein of interest is ovalbumin.


SEQUENCE LISTING (130 sequences)

SEQ ID NO: 1 - protein, 386 aa, Artificial Sequence
  Hen Ovalbumin
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 2 - protein, 388 aa, Artificial Sequence
  Ostrich Ovalbumin
MGSISAANAEFCFDMFKELKIHHVNENIFYSPLSIISALSMVYLGARGNTESQMEKVLHFDEIAGLGGTIHFKCGKSVNIHILFKELLSDITAPTANYSLHIANRLYAEKTRPILPIYLKCVKKLYRAGLETVNFKTASDQARQLINSWVANQTNGQIQDFLEPGSVDLSTVLLLVNAVYFKGMWKKAFREEDTQEVLFRVMEQESKPVQMMCQNGTFKMAEVAAEKIKVLELPYASGELSMLVLLPEDASSLEQLESTISFEKLAKWTSPNMMEKKTVKVYLPRMKLEEKYNLTSVLTALGMTDLFSPAANLSGISAAETLKMSEAVHEASMEVSEAGTEVAGSAGAMGEIKHSPVSAEFRADHPFLLLIRHNPTNSILFFGRYFSP

SEQ ID NO: 3 - protein, 386 aa, Artificial Sequence
  Pelican Ovalbumin
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 4 - protein, 386 aa, Artificial Sequence
  Pigeon Ovalbumin
MASIGAASAEFSFEVFDELKAQYPNQNIIFAPLSILSALSMLYLGARGNTKAQIDKVVHFDKITGSGETVESQCSTSVNVHTSLKDTFIQITKPSDNYSLSFASRLYAEETFQILPEYIQCVKELYKESVETVSFQKAADQARELINSWAESQTNGMIRNILQPGSVDPQTEMVLVNAIYFKGVWAKAFKVEGTQTMPFRVTEQESKPVQMMYQLGSFKVTDLPPEKMRILELPYASGMLSMWVILPYDISGLEQIENAITFEKLQEWTSSNLMEEKTVKVYLPRMKMEEKYNLTSVLMGLGITDLFSSSADLSGISSVERLKVSEAVHQAFVEIDEAGSEAAGIAGAAVKDESASPEFKVDHPFLFLIRHNPTDGILFLARCVSP

SEQ ID NO: 5 - protein, 383 aa, Artificial Sequence
  Quail Ovalbumin
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 6 - protein, 386 aa, Artificial Sequence
  Plover Ovalbumin
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 7 - DNA, 1230 bp, Artificial Sequence
  Hen Ovalbumin
atgtctttccgctccctgctggctttgtctggcctcgtctgcaccggtctggctaacgtgatcagcaagcgcggctccatcggtgccgcttccatggagttctgcttcgatgtcttcaaggagctcaaggtgcaccatgctaacgagaacatcttctactgccccatcgccatcatgagcgccctggctatggtctacctcggtgctaaggactccacccgcacccagatcaacaaggtcgtgcgcttcgataagctgccgggcttcggtgacagcatcgaggcccagtgcggcaccagcgtcaacgtgcactccagcctgcgcgatatcctcaaccagatcaccaagcccaacgacgtctacagcttctctctcgcttcccgactctatgctgaggagcgctaccccatcctgcctgagtacctgcagtgcgtgaaggagctgtaccgcggcggcctggagcctatcaacttccagaccgctgctgatcaggctcgggagctgatcaacagctgggtcgagtcccagaccaacggtatcatccgcaacgtcctgcagccttccagcgtggacagccagaccgccatggtcctggtgaacgctatcgtgttcaagggcctctgggagaaggccttcaaggatgaggacacccaggctatgccgttccgcgtcaccgagcaggagtccaagcccgtgcagatgatgtaccagatcggtctgttccgcgtcgccagcatggcttccgagaagatgaagatcctggagctccctttcgccagcggtaccatgtccatgctcgtcctgctcccggatgaggtgagcggcctggagcagctcgagtccatcatcaacttcgagaagctgaccgagtggaccagcagcaacgtcatggaggagcgcaagatcaaggtgtacctgccccgcatgaagatggaggagaagtacaacctgaccagcgtcctcatggctatgggtatcaccgacgtgttctccagctccgccaacctgtccggcatcagctccgctgagagcctcaagatcagccaggctgtgcatgctgctcatgctgagatcaatgaggctggtcgcgaggtcgtgggttctgctgaggctggcgtcgatgctgctagcgtgtccgaggagttccgggctgaccaccccttcctgttctgcatcaagcatatcgccaccaacgctgtcctcttcttcggccgctgcgtgtccccttga

SEQ ID NO: 8 - DNA, 1236 bp, Artificial Sequence
  Ostrich Ovalbumin
atgtctttccgcagcctgctggctctgtctggcctggtctgcaccggcctggctaacgtgatcagcaagcgcggctccatctctgctgctaatgctgagttctgcttcgatatgttcaaggagctgaagatccaccatgtgaacgagaacatcttctactcccctctgagcatcatctccgccctgagcatggtgtacctcggcgctcgcggtaacaccgagtcccagatggagaaggtcctgcacttcgatgaaatcgctggcctgggcggcaccatccacttcaagtgcggcaagagcgtcaacatccatatcctcttcaaggagctgctctccgacatcaccgcccctaccgctaactacagcctgcatatcgccaaccgcctctacgctgagaagacccgccccatcctgcctatctacctcaagtgcgtgaagaagctgtaccgcgccggcctggaaaccgtcaacttcaagaccgcctccgatcaggctcgccagctgatcaacagctgggtcgctaaccagaccaacggtcagatccaggatttcctggagcctggctccgtcgacctcagcaccgtgctgctcctggtcaacgccgtgtacttcaagggcatgtggaagaaggctttccgcgaggaagatacccaggaagtcctcttccgcgtgatggagcaggagtccaagccggtgcagatgatgtgccagaacggtaccttcaagatggccgaggtcgccgctgagaagatcaaggtgctggagctcccttacgcctccggcgagctgagcatgctcgtcctcctgccggaggatgcttccagcctggagcagctcgagtccaccatcagcttcgagaagctggccaagtggaccagccccaacatgatggagaagaagaccgtcaaggtgtacctgcctcgcatgaagctcgaggagaagtacaacctgaccagcgtcctgaccgctctcggtatgaccgacctgttcagccctgctgctaacctctctggtatcagcgccgctgaaaccctcaagatgtccgaggccgtccacgaggcttccatggaagtctccgaagctggtaccgaggtcgctggttctgctggtgctatgggcgagatcaagcactctccggtcagcgctgagttccgagctgaccacccgttcctcctgctcatccgccataaccccaccaactccatcctcttcttcggccgctacttcagcccctag

SEQ ID NO: 9 - DNA, 1230 bp, Artificial Sequence
  Pelican Ovalbumin
atgtctttccgctccctgctggctctgtctggcctcgtctgcaccggtctggctaacgtgatcagcaagcgcggctccatcggtgctgcttccaccgagttctgcttcgatgtcttcaaggagctgaaggtccagcacgtgaacgagaacatcttctacagccccctctccatcatcagcgccctgtccatggtgtatctgggtgctcgggagaacacccgagctcagatcgataaggtcgtgcacttcgacaagatcaccggcttcggcgagcctatcgagagccagtgcggcatcagcgtctccgtgcataccagcctgaaggatatgatcacccagatcaccaagccgagcgacaactacagcctgtccttcgcctcccgcctctacgctgaggaaacctaccccatcctgcctgagtacctgcagtgcgtcaaggagctgtacaagggcggcctggaaaccatctctttccagaccgctgctgatcaggctcgagagctcatcaactcctgggtcgagaaccagaccaacggtatgatcaagaacatcctccagcctggctccgtcgatccccagaccgagatggtcctcgtgaacgctgtgtacttcaagggcatgtgggagaaggccttcaaggatgaggacacccaggctgtccccttccgcatgaccgagcaggagtccaagcctgtgcagatgatgtaccagatcggcagcttcaaggtcgccgtgatggcttccgagaagatcaagatcctggagctcccgtacgccagcggcgagctgtccatgctcgtcctgctccccgatgacgtgtccggcctggagcagctcgaaaccgccatcaccctggataagctcaccgagtggaccagcagcaacgctatggaggagcgcaagatgaaggtctacctgccccgcatgaagatcgagaagaagtacaacctcaccagcgtgctgatcgccctcggtatgaccgacctgttctccagctccgccaacctctccggcatcagctccgctgagagcctgaagatgtccgaggccatccatgaggctttcctcgagatctatgaggctggtagcgaggtcgtgggttccaccgaggctggcatggaagtcaccagcgtgtccgaggagttccgcgctgaccaccctttcctgttcctcatcaagcataacccgaccaacagcatcctgttcttcggccgctgcctctccccttag

SEQ ID NO: 10 - DNA, 1230 bp, Artificial Sequence
  Pigeon Ovalbumin
atgtctttccgctccctgctggctttgtctggcctcgtctgcaccggcctggctaacgtgatctccaagcgggctagcatcggtgctgcttctgctgagttcagcttcgaggtcttcgatgagctgaaggcccagtaccccaaccagaacatcatcttcgctcctctgagcatcctctctgctctgagcatgctgtacctgggtgctcgcggtaacaccaaggctcagatcgataaggtcgtgcacttcgacaagatcaccggctccggtgaaaccgtggagagccagtgcagcaccagcgtcaacgtgcataccagcctcaaggataccttcatccagatcaccaagccgtccgacaactacagcctctccttcgccagccgcctgtacgctgaggaaaccttccagatcctccccgagtacatccagtgcgtcaaggagctgtacaaggagtccgtcgaaaccgtctccttccagaaggccgctgatcaggctcgcgagctgatcaactcctgggccgagagccagaccaacggcatgatccgcaacatcctccagccgggtagcgtggatccccagaccgagatggtcctggtgaacgccatctacttcaagggcgtctgggccaaggctttcaaggtggagggtacccagaccatgccgttccgcgtcaccgagcaggagtccaagcccgtgcagatgatgtaccagctcggcagcttcaaggtcaccgatctgccccctgagaagatgcgcatcctggagctcccttacgcttccggcatgctcagcatgtgggtgatcctgccgtacgacatctccggcctggagcagatcgagaacgccatcaccttcgagaagctccaggagtggaccagcagcaacctgatggaggagaagaccgtcaaggtgtacctgccccgcatgaagatggaggagaagtacaacctgaccagcgtcctcatgggcctgggtatcaccgatctcttctcctcttctgctgacctgtctggtatcagctccgtcgagcgactgaaggtcagcgaggctgtgcaccaggctttcgtggagatcgatgaggctggttctgaggctgctggtatcgctggtgctgctgtcaaggatgagagcgcttcccctgagttcaaggtggaccacccgttcctgttcctcatccgccataaccccaccgacggtatcctgttcctcgcccgctgcgtctccccttag

SEQ ID NO: 11 - DNA, 1221 bp, Artificial Sequence
  Quail Ovalbumin
atgtctttccgcagcctgctggctttgtctggcctcgtctgcaccggtctggctaacgtgatctccaagcgcggcagcatcggtgccgctagcatggagttctgcttcgatgtcttcaaggagctgaaggtgcaccatgccaacgacaacatgctctactcccccttcgccatcctgagcaccctcgctatggtcttcctgggtgccaaggattccacccgcacccagatcaacaaggtcgtgcacttcgataagctccctggcttcggtgactccatcgaggctcagtgcggcaccagcgccaacgtgcattccagcctgcgcgatatcctcaaccagatcaccaagcagaacgacgcctactccttcagcctggcctcccgcctctacgctcaggaaacctacaccgtcgtgcctgagtacctgcagtgcgtcaaggagctgtaccgcggcggcctggagagcgtgaatttccagaccgctgctgaccaggctcgcggtctgatcaacgcctgggtcgagtcccagaccaacggcatcatccgcaacatcctccagccctccagcgtggatagccagaccgctatggtcctggtgaacgccatcgctttcaagggcctgtgggagaaggccttcaaggctgaggacacccagaccatccccttccgcgtcaccgagcaggagtccaagcctgtgcagatgatgcaccagatcggcagcttcaaggtcgcctccatggctagcgagaagatgaagatcctggagctcccgttcgcctccggtaccatgagcatgctggtcctgctccccgatgacgtgtccggcctggagcagctcgagtccaccatcagcttcgagaagctcaccgagtggaccagcagctccatcatggaggagcgcaaggtcaaggtgtacctgccgcgcatgaagatggaggagaagtacaacctcaccagcctgctcatggctatgggtatcaccgacctgttctcctccagcgccaacctctccggcatctccagcgtcggtagcctgaagatcccccaggctgtgcatgccgcttacgccgagatcaatgaggctggtcgggatgtggtgggttctgctgaggctggcgtcgatgctaccgaggagttccgcgccgaccaccctttcctcttctgcgtcaagcatatcgaaaccaacgccatcctgctcttcggccgctgcgtgagcccgtag

SEQ ID NO: 12 - DNA, 1230 bp, Artificial Sequence
  Plover Ovalbumin
atgtctttccgctccctgctggctctgtctggcctcgtctgcaccggcctggctaacgtgatcagcaagcgcggctccatcggtgctgcttccaccgagttctgcttcgatgtcttcaaggagctcaaggtccagcacgtgaacgagaacatcttctactcccctctgaccatcatctctgctctctccatggtgtatctgggtgctcgggagaacacccgagctcagatcgataaggtcgtgcacttcgacaagatcccgggcttcggtgacaccaccgagagccagtgcggtaccagcgtctccgtgcataccagcctcaaggatatgttcacccagatcaccaagcccagcgacaactacagcgtctccttcgcctcccgcctgtacgctgaggaaacctaccccatcctccctgagttcctggagtgcgtgaaggagctctacaagggcggtctggagagcatctctttccagaccgctgctgatcaggctcgagagctcatcaacagctgggtcgagtcccagaccaacggcatgatcaagaacatcctccagccgggtagcgtggactcccagaccgagatggtcctggtgaacgccatctacttcaagggcatgtgggagaaggctttcaaggatgaggacacccagaccgtccctttccgcatgaccgagcaggaaaccaagccggtgcagatgatgtaccagatcggtaccttcaaggtcgccgtgatgcccagcgagaagatgaagatcctggagctcccttacgcttccggcgagctgtgcatgctcgtcatgctgcctgatgacgtgtccggcctggaggagctggagtccagcatcaccgtcgagaagctgatggagtggaccagcagcaacatgatggaggagcgcaagatgaaggtcttcctcccgcgcatgaagatcgaggagaagtacaacctgaccagcgtcctcatggccctgggcatgaccgatctcttctccagctccgccaacctgagcggtatcagctccgctgagcccctcaagatgtccgaggccgtccatgaggctttcatcgagatctacgaagctggtagcgaggtcgtgggttccaccggcgctggtatggagatcaccagcgtgtccgaggagttccgcgctgaccacccgttcctgttcctcatcaagcataaccccaccaacagcatcctgttcttcggccgctgcgtctccccttag

SEQ ID NO: 13 - DNA, 2579 bp, Artificial Sequence
  pUC57-kan
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 14 - protein, 24 aa, Artificial Sequence
  GLA pre-pro sequence
MSFRSLLALSGLVCTGLANVISKR

SEQ ID NO: 15 - protein, 510 aa, Artificial Sequence
  Truncated GLA and synthetic linker
MSFRSLLALSGLVCTGLANVISKRATWDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEVNGSSFFTIAVQHRALVEGSAFATAVGSSCSWCDSQAPEILCYLQSFWTGSFILANFDSSRSAKDANTLLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDATGTYSSSSSTYSSIVDAVKTFADGFVSIVETHAASNGSMSEQYDKSDGEQLSARDLTWSYAALLTANNRRNVVPSASWGETSASSVPGTCAATSAIGTYSSVTVTSWPSIVATGGTTTTASKSVISKR

SEQ ID NO: 16 - DNA, 1782 bp, Artificial Sequence
  Truncated GLA and synthetic linker DNA sequence
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaccattgagaactacatctccgcccaggcaattgtccagggtatcagtaacccctctggtgatctgtccagcggcgctggcctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggcctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacttctgccagcagcgtgcccggcacctgtgcggccacatctgccattggtacctacagcagtgtgactgtcacctcgtggccgagtatcgtggctactggcggcaccactacgacggcttccaagtctgtcatctccaagcgc

SEQ ID NO: 17 - DNA, 36 bp, Artificial Sequence
  Primer 406
aaaggtctcgggagcctctcgtatgcagaggaaatc

SEQ ID NO: 18 - DNA, 34 bp, Artificial Sequence
  Primer 408
aaaggtctcgcatttgctgaggtgtaatgatgct

SEQ ID NO: 19 - DNA, 44 bp, Artificial Sequence
  Primer 409
aaaggtctccgcttacaatcaatccatttcgctatagttaaagg

SEQ ID NO: 20 - DNA, 38 bp, Artificial Sequence
  Primer 410
aaaggtctcaatggatttagtggcagatggcatcacag

SEQ ID NO: 21 - DNA, 3519 bp, Artificial Sequence
  pCR Blunt II-TOPO vector
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 22 - DNA, 2174 bp, Artificial Sequence
  pGGA
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcgagaccgctttccagatctgataacttgtggtctcaccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 23 - DNA, 4547 bp, Artificial Sequence
  pBZ0024
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttaaaggtctcgggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgcgagacctttaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 24 - DNA, 4147 bp, Artificial Sequence
  pBZ0025
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttaaaggtctccgcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattgagacctttaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 25 - DNA, 4978 bp, Artificial Sequence
  pBZ0028
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtctttccgcagcctgctggctctgtctggcctggtctgcaccggcctggctaacgtgatcagcaagcgcggctccatctctgctgctaatgctgagttctgcttcgatatgttcaaggagctgaagatccaccatgtgaacgagaacatcttctactcccctctgagcatcatctccgccctgagcatggtgtacctcggcgctcgcggtaacaccgagtcccagatggagaaggtcctgcacttcgatgaaatcgctggcctgggcggcaccatccacttcaagtgcggcaagagcgtcaacatccatatcctcttcaaggagctgctctccgacatcaccgcccctaccgctaactacagcctgcatatcgccaaccgcctctacgctgagaagacccgccccatcctgcctatctacctcaagtgcgtgaagaagctgtaccgcgccggcctggaaaccgtcaacttcaagaccgcctccgatcaggctcgccagctgatcaacagctgggtcgctaaccagaccaacggtcagatccaggatttcctggagcctggctccgtcgacctcagcaccgtgctgctcctggtcaacgccgtgtacttcaagggcatgtggaagaaggctttccgcgaggaagatacccaggaagtcctcttccgcgtgatggagcaggagtccaagccggtgcagatgatgtgccagaacggtaccttcaagatggccgaggtcgccgctgagaagatcaaggtgctggagctcccttacgcctccggcgagctgagcatgctcgtcctcctgccggaggatgcttccagcctggagcagctcgagtccaccatcagcttcgagaagctggccaagtggaccagccccaacatgatggagaagaagaccgtcaaggtgtacctgcctcgcatgaagctcgaggagaagtacaacctgaccagcgtcctgaccgctctcggtatgaccgacctgttcagccctgctgctaacctctctggtatcagcgccgctgaaaccctcaagatgtccgaggccgtccacgaggcttccatggaagtctccgaagctggtaccgaggtcgctggttctgctggtgctatgggcgagatcaagcactctccggtcagcgctgagttccgagctgaccacccgttcctcctgctcatccgccataaccccaccaactccatcctcttcttcggccgctacttcagcccctaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 26 - DNA, 4972 bp, Artificial Sequence
  pBZ0029
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtctttccgctccctgctggctctgtctggcctcgtctgcaccggcctggctaacgtgatcagcaagcgcggctccatcggtgctgcttccaccgagttctgcttcgatgtcttcaaggagctcaaggtccagcacgtgaacgagaacatcttctactcccctctgaccatcatctctgctctctccatggtgtatctgggtgctcgggagaacacccgagctcagatcgataaggtcgtgcacttcgacaagatcccgggcttcggtgacaccaccgagagccagtgcggtaccagcgtctccgtgcataccagcctcaaggatatgttcacccagatcaccaagcccagcgacaactacagcgtctccttcgcctcccgcctgtacgctgaggaaacctaccccatcctccctgagttcctggagtgcgtgaaggagctctacaagggcggtctggagagcatctctttccagaccgctgctgatcaggctcgagagctcatcaacagctgggtcgagtcccagaccaacggcatgatcaagaacatcctccagccgggtagcgtggactcccagaccgagatggtcctggtgaacgccatctacttcaagggcatgtgggagaaggctttcaaggatgaggacacccagaccgtccctttccgcatgaccgagcaggaaaccaagccggtgcagatgatgtaccagatcggtaccttcaaggtcgccgtgatgcccagcgagaagatgaagatcctggagctcccttacgcttccggcgagctgtgcatgctcgtcatgctgcctgatgacgtgtccggcctggaggagctggagtccagcatcaccgtcgagaagctgatggagtggaccagcagcaacatgatggaggagcgcaagatgaaggtcttcctcccgcgcatgaagatcgaggagaagtacaacctgaccagcgtcctcatggccctgggcatgaccgatctcttctccagctccgccaacctgagcggtatcagctccgctgagcccctcaagatgtccgaggccgtccatgaggctttcatcgagatctacgaagctggtagcgaggtcgtgggttccaccggcgctggtatggagatcaccagcgtgtccgaggagttccgcgctgaccacccgttcctgttcctcatcaagcataaccccaccaacagcatcctgttcttcggccgctgcgtctccccttaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 27 - DNA, 4972 bp, Artificial Sequence
  pBZ0030
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtctttccgctccctgctggctctgtctggcctcgtctgcaccggtctggctaacgtgatcagcaagcgcggctccatcggtgctgcttccaccgagttctgcttcgatgtcttcaaggagctgaaggtccagcacgtgaacgagaacatcttctacagccccctctccatcatcagcgccctgtccatggtgtatctgggtgctcgggagaacacccgagctcagatcgataaggtcgtgcacttcgacaagatcaccggcttcggcgagcctatcgagagccagtgcggcatcagcgtctccgtgcataccagcctgaaggatatgatcacccagatcaccaagccgagcgacaactacagcctgtccttcgcctcccgcctctacgctgaggaaacctaccccatcctgcctgagtacctgcagtgcgtcaaggagctgtacaagggcggcctggaaaccatctctttccagaccgctgctgatcaggctcgagagctcatcaactcctgggtcgagaaccagaccaacggtatgatcaagaacatcctccagcctggctccgtcgatccccagaccgagatggtcctcgtgaacgctgtgtacttcaagggcatgtgggagaaggccttcaaggatgaggacacccaggctgtccccttccgcatgaccgagcaggagtccaagcctgtgcagatgatgtaccagatcggcagcttcaaggtcgccgtgatggcttccgagaagatcaagatcctggagctcccgtacgccagcggcgagctgtccatgctcgtcctgctccccgatgacgtgtccggcctggagcagctcgaaaccgccatcaccctggataagctcaccgagtggaccagcagcaacgctatggaggagcgcaagatgaaggtctacctgccccgcatgaagatcgagaagaagtacaacctcaccagcgtgctgatcgccctcggtatgaccgacctgttctccagctccgccaacctctccggcatcagctccgctgagagcctgaagatgtccgaggccatccatgaggctttcctcgagatctatgaggctggtagcgaggtcgtgggttccaccgaggctggcatggaagtcaccagcgtgtccgaggagttccgcgctgaccaccctttcctgttcctcatcaagcataacccgaccaacagcatcctgttcttcggccgctgcctctccccttaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 28 - DNA, 4972 bp, Artificial Sequence
  pBZ0031
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtctttccgctccctgctggctttgtctggcctcgtctgcaccggcctggctaacgtgatctccaagcgggctagcatcggtgctgcttctgctgagttcagcttcgaggtcttcgatgagctgaaggcccagtaccccaaccagaacatcatcttcgctcctctgagcatcctctctgctctgagcatgctgtacctgggtgctcgcggtaacaccaaggctcagatcgataaggtcgtgcacttcgacaagatcaccggctccggtgaaaccgtggagagccagtgcagcaccagcgtcaacgtgcataccagcctcaaggataccttcatccagatcaccaagccgtccgacaactacagcctctccttcgccagccgcctgtacgctgaggaaaccttccagatcctccccgagtacatccagtgcgtcaaggagctgtacaaggagtccgtcgaaaccgtctccttccagaaggccgctgatcaggctcgcgagctgatcaactcctgggccgagagccagaccaacggcatgatccgcaacatcctccagccgggtagcgtggatccccagaccgagatggtcctggtgaacgccatctacttcaagggcgtctgggccaaggctttcaaggtggagggtacccagaccatgccgttccgcgtcaccgagcaggagtccaagcccgtgcagatgatgtaccagctcggcagcttcaaggtcaccgatctgccccctgagaagatgcgcatcctggagctcccttacgcttccggcatgctcagcatgtgggtgatcctgccgtacgacatctccggcctggagcagatcgagaacgccatcaccttcgagaagctccaggagtggaccagcagcaacctgatggaggagaagaccgtcaaggtgtacctgccccgcatgaagatggaggagaagtacaacctgaccagcgtcctcatgggcctgggtatcaccgatctcttctcctcttctgctgacctgtctggtatcagctccgtcgagcgactgaaggtcagcgaggctgtgcaccaggctttcgtggagatcgatgaggctggttctgaggctgctggtatcgctggtgctgctgtcaaggatgagagcgcttcccctgagttcaaggtggaccacccgttcctgttcctcatccgccataaccccaccgacggtatcctgttcctcgcccgctgcgtctccccttaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 29 - DNA, 4963 bp, Artificial Sequence
  pBZ0032
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtctttccgcagcctgctggctttgtctggcctcgtctgcaccggtctggctaacgtgatctccaagcgcggcagcatcggtgccgctagcatggagttctgcttcgatgtcttcaaggagctgaaggtgcaccatgccaacgacaacatgctctactcccccttcgccatcctgagcaccctcgctatggtcttcctgggtgccaaggattccacccgcacccagatcaacaaggtcgtgcacttcgataagctccctggcttcggtgactccatcgaggctcagtgcggcaccagcgccaacgtgcattccagcctgcgcgatatcctcaaccagatcaccaagcagaacgacgcctactccttcagcctggcctcccgcctctacgctcaggaaacctacaccgtcgtgcctgagtacctgcagtgcgtcaaggagctgtaccgcggcggcctggagagcgtgaatttccagaccgctgctgaccaggctcgcggtctgatcaacgcctgggtcgagtcccagaccaacggcatcatccgcaacatcctccagccctccagcgtggatagccagaccgctatggtcctggtgaacgccatcgctttcaagggcctgtgggagaaggccttcaaggctgaggacacccagaccatccccttccgcgtcaccgagcaggagtccaagcctgtgcagatgatgcaccagatcggcagcttcaaggtcgcctccatggctagcgagaagatgaagatcctggagctcccgttcgcctccggtaccatgagcatgctggtcctgctccccgatgacgtgtccggcctggagcagctcgagtccaccatcagcttcgagaagctcaccgagtggaccagcagctccatcatggaggagcgcaaggtcaaggtgtacctgccgcgcatgaagatggaggagaagtacaacctcaccagcctgctcatggctatgggtatcaccgacctgttctcctccagcgccaacctctccggcatctccagcgtcggtagcctgaagatcccccaggctgtgcatgccgcttacgccgagatcaatgaggctggtcgggatgtggtgggttctgctgaggctggcgtcgatgctaccgaggagttccgcgccgaccaccctttcctcttctgcgtcaagcatatcgaaaccaacgccatcctgctcttcggccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 30 - DNA, 1230 bp, Artificial Sequence
  Hen Ovalbumin cDNA odon optimized for glaA gene (A. niger)
atgtcgttccgatctctactcgccctgagcggcctcgtctgcacagggttggcaaatgtgatttccaagcgcggctctattggagcggcctccatggaattctgcttcgacgtgttcaaggagttgaaggtccaccacgctaacgaaaatattttctactgtccgattgctattatgtccgccctcgcgatggtatacctgggcgccaaggactctacccgaactcagatcaataaggtcgtgagattcgataagctgcctggattcggtgattcgatcgaagcgcagtgcggcacctccgtgaatgtccacagcagtctgcgagatattctgaaccaaatcactaagcccaacgatgtgtatagtttcagcctggcctctcgtctctacgcagaagagcgctaccctattctgcccgaatacttgcagtgcgtgaaggaactctaccgagggggcctggagcccataaacttccagacggctgctgaccaagcccgggagctcatcaacagttgggttgagtcacagactaacggtatcattcgcaacgtgctccagcctagcagtgttgacagccagaccgcgatggtgctagtcaacgccattgtgttcaagggtctctgggagaaggcctttaaggacgaggatacacaggctatgcccttccgcgtgaccgaacaggaaagcaagcccgtccagatgatgtaccagattggccttttccgtgtcgcaagcatggcgagcgaaaagatgaagatcctggagctcccgtttgcctcgggtaccatgtccatgctcgttctccttccggacgaagtgagcggactggagcagctggagagcatcatcaacttcgagaagttgacggagtggactagctctaacgttatggaggagcgcaagattaaggtgtatctcccccggatgaagatggaggagaagtacaacctgacctctgtgctgatggctatgggcattaccgatgtgtttagttcctccgctaacttgtccgggatctcgtcggctgagtcactcaagatttcccaagctgtccacgctgctcacgctgagatcaacgaggctggtcgtgaagtcgtcggctcggcagaagctggcgtcgatgcggcatctgtctctgaggagttccgcgccgaccacccgtttctattctgcatcaagcacatcgcaaccaatgccgttctgttcttcggcaggtgtgtgtcgccgtag

SEQ ID NO: 31 - DNA, 2710 bp, Artificial Sequence
  pUC57
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 32 - DNA, 3940 bp, Artificial Sequence
  BZESCO.21
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatgtcgttccgatctctactcgccctgagcggcctcgtctgcacagggttggcaaatgtgatttccaagcgcggctctattggagcggcctccatggaattctgcttcgacgtgttcaaggagttgaaggtccaccacgctaacgaaaatattttctactgtccgattgctattatgtccgccctcgcgatggtatacctgggcgccaaggactctacccgaactcagatcaataaggtcgtgagattcgataagctgcctggattcggtgattcgatcgaagcgcagtgcggcacctccgtgaatgtccacagcagtctgcgagatattctgaaccaaatcactaagcccaacgatgtgtatagtttcagcctggcctctcgtctctacgcagaagagcgctaccctattctgcccgaatacttgcagtgcgtgaaggaactctaccgagggggcctggagcccataaacttccagacggctgctgaccaagcccgggagctcatcaacagttgggttgagtcacagactaacggtatcattcgcaacgtgctccagcctagcagtgttgacagccagaccgcgatggtgctagtcaacgccattgtgttcaagggtctctgggagaaggcctttaaggacgaggatacacaggctatgcccttccgcgtgaccgaacaggaaagcaagcccgtccagatgatgtaccagattggccttttccgtgtcgcaagcatggcgagcgaaaagatgaagatcctggagctcccgtttgcctcgggtaccatgtccatgctcgttctccttccggacgaagtgagcggactggagcagctggagagcatcatcaacttcgagaagttgacggagtggactagctctaacgttatggaggagcgcaagattaaggtgtatctcccccggatgaagatggaggagaagtacaacctgacctctgtgctgatggctatgggcattaccgatgtgtttagttcctccgctaacttgtccgggatctcgtcggctgagtcactcaagatttcccaagctgtccacgctgctcacgctgagatcaacgaggctggtcgtgaagtcgtcggctcggcagaagctggcgtcgatgcggcatctgtctctgaggagttccgcgccgaccacccgtttctattctgcatcaagcacatcgcaaccaatgccgttctgttcttcggcaggtgtgtgtcgccgtagatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 33 - DNA, 1830 bp, Artificial Sequence
  Truncated GLA
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggtctcgtcctcaagaccctcgtcgatctcttccgaaatggagataccagtctcctctccaccattgagaactacatctccgcccaggcaattgtccagggtatcagtaacccctctggtgatctgtccagcggcgctggtctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggtctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacctctgccagcagcgtgcccggcacctgtgcggccacatctgccattggtacctacagcagtgtgactgtcacctcgtggccgagtatcgtggctactggcggcaccactacgacggctacccccactggatccggcagcgtgacctcgaccagcaagaccaccgcgactgctagcgatgacgatgacaag

SEQ ID NO: 34 - DNA, 36 bp, Artificial Sequence
  Primer 276
gatatctagacctctcgtatgcagaggaaatctccc

SEQ ID NO: 35 - DNA, 48 bp, Artificial Sequence
  Primer 286
gctccaatagagcccttgtcatcgtcatcgctagcagtcgcggtggtc

SEQ ID NO: 36 - DNA, 46 bp, Artificial Sequence
  Primer 277
gtagagatcggaacgacattgctgaggtgtaatgatgctggggatg

SEQ ID NO: 37 - DNA, 39 bp, Artificial Sequence
  Primer 280
ggcaggtgtgtgtcgccgtagacaatcaatccatttcgc

SEQ ID NO: 38 - DNA, 30 bp, Artificial Sequence
  Primer 281
ctgcactcgagggaagttgggtttgcgtgc

SEQ ID NO: 39 - DNA, 44 bp, Artificial Sequence
  Primer 278
cagcatcattacacctcagcaatgtcgttccgatctctactcgc

SEQ ID NO: 40 - DNA, 39 bp, Artificial Sequence
  Primer 279
gcgaaatggattgattgtctacggcgacacacacctgcc

SEQ ID NO: 41 - DNA, 49 bp, Artificial Sequence
  Primer 287
caccgcgactgctagcgatgacgatgacaagggctctattggagcggcc

SEQ ID NO: 42 - DNA, 5424 bp, Artificial Sequence
  pUC57-Brick
gcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatccgcataggctgaggggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggaccctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtgacatggttcgtacaaaccaaatgcggtgttcttgtctggcaaagaggccaaggacgcagatggcaacaaacccaaggaacctgggataacggaggcttcatcggagataatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtctcctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaatagcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcact

SEQ ID NO: 43 - DNA, 13093 bp, Artificial Sequence
  BZESCO.22
agtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgctatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggagaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatattatctccgatgaagcctccgttatcccaggttccttgggtttgttgccatctgcgtccttggcctctttgccagacaagaacaccgcatttggtttgtacgaaccatgtcacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaaagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatcccgccagggttttcccagtcacgacgttgtaaaacgacggccagtgaattcgagctcggtacctcgcgaatgcatctagacatgtgtggagagggaggttagtcaagttgggtcagtcaccagacggtgagagagagagagaaagagagagaacttggaggcgagaacggaggggagagagagttgagagtcacagccctattaggcagctccaacatctgcactctgccccagcagaagcagcagtagcagaagcaaatatctcagtcagcaagtcagacggtgtctcagtcggacccagactcttagtaccagctggaagaaggacaagactactactgttacttactccgtactacttttcccacttcccagccccccgctagtgaccttcggatgtgaaccgcggctgttcggcggcgaaacataaaggcgcgcattccgcggaccccaattgctcggctatcacgtgatgcctagtcaatcacctcactttgcgccatcatggcggatgcagcaatcttgatgacttttccatagactaactgctcactagtgaaattagcatcctctacgtagctgctccagtcagcactaatcatcgcgtcattcaggaacactcctcccttttcaatgcctcatgatagtatttctccaaggtcatacaaaagattacagcaacataaggattatatacagctggatttcccacaacattcgaggcaacttttgcgcgcaagccataatgcaaacggtgaaaatggagggagaaaaagaaaaaaaaaaaaaagataaggaggtccaagcccagctagaggtcaatttcagcccaggaccgctgtacaggactgagtggagccattatagaatgctcttcgcggtcctgcagcatgctagtccagggctctctcgtcgggagttgaccgctgcagcttgtctccggggcgcagaccggcacccgctagcgttttagaacctgccagaaacgccatggatatcaagagatcagaaatgggtgggcacttttgcagctttcaaataacaggcatggcaactcatcgctagcgtccgcggcatcacttagaaaccgggaactttccgcttcttcatcagagccttgcgctccttctccttggctgtattgcgaaaaattatattagcattcattcacccaatcagaaaaagaattatagtgcataccttcgtcatagacaaggaactcgtgcagggcacagccagcgggcaccatggggagcacaggcaccttgcgatcagtaaacacttccagaagggcagggccgtcctgctcgatcagccacttgagcttcgcctcaacctccgagggcttggtgcagcggtcggcagcaacacccatggactgggccatgggaacgaagtcggggttcttctggtgagtgtgcgagtaccggtcctcgtagaacaggttctgccattgcgtcaccataccctgttcctcgttgttcaggaggagaaccttcacaccaatgttgaactgagcagcagtggtgagctcagtcagggtcatgttgaacgaggcatcaccatcgatatcaacgacaagagcgtcagggcgggcaaccttggcaccaatggcagcggggagaccgtatcccatagttccaagaccaccagaggtgatcatggtacgggggtggcgccagcggaagtgctgagcagcccacatctgatgctgaccgacaccagtagtgatgacagtgcggtccttcatgtgagcggtgagatcgctgagcttctcaatcacagcctggggcttgatgggtccttcagcagtctgcttctcataaagggagaaggggaagcgagccttccagtcgttgatctgctcgaaccactccttgcgctcagcaacgggctcgacgtggggcaggaggagacggatgttctcagcacagtcaccctcaacagcctcgttggcctggacaaccttgttgatgttcttgggcatgatttcaaagtggacgataccaccacggttctccgaggcagcaagcttggcctggggggcaaacttggagatgttaccagtcacatggtcatcgaagcgagcaccgatagcgatgatgagatcagcctcctgcatggccatgttggcataggcagatccgtgcattcccagcatgtgcagggccttggggtccgtttcgtcgaacccacccagaccctggagagtggtggtgaccggaatgcaagccttgtcggccagctccttcaagatctggggtccttcagggcgagcaaggagaccctgacccacatacaggatgggcttcttggaaacgttcacgaggcgggccacacggttgatggtaccctgcagctgctgcatgctcagttcgcgggcggccatggtcgcagcgctggggagagagggcagggtgctgttcatggggatgggtttacggagaataccggcggtaatatcctcgggcagatcgacgaggacaggtccggggcggccgctggtggcgatttcgaaggcctcctggatgcgacgggggagttcagcgacggacttgaccatgacgttccacttggtgcaagcgcgagagataccgataacatcggcttcctggaagtcatcggtaccgatcaggctggtaggcacctgaccgcagaagacgaccatgggggtaccgtccgagagggcatcctgcatgggggtgataacgttggtggctccagggccggaggtgacaaggacgacaccgggctttccagaggcacgggcgtagccttcggccatgtgtccggcgccctgttcatgtctcggaagaacgaagtcgaagtgtttggagttgtaaatagcatcgaagacgggaagaatggcacctccagggtaaccaactaaaagtagtgggtcaatcttacggtcttgaagaacatcgggaaaccgagaaaaaagacgcgtgactcacagacatgcttgacgccaagacgcagcatcatctcatgaaagatctctcctccactgagtccgacaaaactgtttacgctccaagaaccaggtcagctctactcccatgagtcatctatgttgttgaagggactgcatacgagtcgtccagttcggggacctggcgattttgcagtggcgagacctcattgcggtgaggttcctggttgaaggcagggctggggacaggtcttgtgcctctggaaattcacagacatgttagcaacagtcattgacagcccagtagctatctggatgctaattgtagataatactcactcagccggggcagttgcggtgctctggaatctcttctgagctgaaaagcggtggggcgcggtcgcagcaacggaagcagtggtaaaagtcattctgccggaggcgatatacctctgataatgcagagcgcgcatggcgcttttagaaggtctcataggcatcatcttgatggcggatgactgccaggcctggggaacagagaaaggggagtgctcacaacggggaaaaagaatgggaccaggaagtgtgagtaagagatccgttccttaaggcaggagcgaagactcagacttcggggacagttatggttgcattctagcctgtcctggtctagcgccgttggagtcgaataatctcggtcttcccattggctacggatactccactgctccgatcatcggccttctggacgcgaaagcggtcacctgattgggatgcaagaccctcgtttcgtcctggtctttaccagagcaacgccttgcaaaattgcaataatacaggcgagggatgttgatgatggaaactaacgttgcacaacgacgtatatttctttgctattaattgtcatggtgcggccttttgtgtcaaggttccaactgctatacgatcctgtaatcaactagcccggtttactgctgacatactcctagtcctcatagcaaccaagagacataagggataaaaatcgattacaactcacggactacatcatttagtaattagggcttctataaaatgccactctaaccatacctccatcgatctgtggtaatcacagactttttgagatttctacacatactcctaccgcgggtgccggtccgaatacggctcgctaatgcatctccatcacatgcaggtaaccggcatgaaaagaaagcaagtgttgtatgattttacacccgctactatttactggtgcagtaattctccgtggaggggctgtctatttactatgaagatgactcactcgtaacattggccagtgattcctgaacaatgcctctaattcccaaatcctaggaacataggcacctcggccttaaacttcctttattttcatgaagctgggccgagctcagtggagcatatacatcagggctgcctgcagactgtttgttcggccttgcgtcagatatgcagagtttctgtcgcaatgagaacctcgagggaagttgggtttgcgtgcagagaccgatggctcctcatccagcagtacctgctgctgcacagcgagggcatcgcatcccacgaatagggccaacaaagccggcaagaacatcatgatgggagcgacaattccgtatcaatctcccttgcacgatgttggttgtcactcaccgacactccgtcaccaagatcactattaaaacaagagtgagttcaaggttgcgatcaagatagcgattgcgcagcaacggcacgggataacgccatagctctgatggagccgatcagaccagtaggtaaactagtcagtcagcgttggactggagctgcagagagagttgaacctggacgccgcgcaaaaagcaaagacgcgcctcgtgggcggtggatcaatgatcggatttagtggcagatggcatcacaggcggccaatgaccaccgggccaactggccccgacattccagcaatactgcctaattgactccaccatgcatctcggctattattgaactgggtttgatggatggggaccctcttggaattgtcaaagattttgaagcgaagacgatctattggacggtagagatatactcttgatttagtcgttgggaggcccctggggaaagcaatgatggggaatgttgctgctccactgtggacctcggctatggaattacgtgcttggatctaagatgagctcatggctatgcattgaatgacagtgatatcagcagagcaagcagagaaggatggaatgctaattttctagtgctttgtgcaagggtaaatcagggactgtctgtctggtcttctacacgaaggaaagaccatggctttcacggtgtctgtatttccggatatcctcaattccgtcggtcgattacaatcacatgacttggcttccatttcactactattatgcacacccactacatacatgatcatataaccaattgccctcatccccatcctttaactatagcgaaatggattgattgtctacggcgacacacacctgccgaagaacagaacggcattggttgcgatgtgcttgatgcagaatagaaacgggtggtcggcgcggaactcctcagagacagatgccgcatcgacgccagcttctgccgagccgacgacttcacgaccagcctcgttgatctcagcgtgagcagcgtggacagcttgggaaatcttgagtgactcagccgacgagatcccggacaagttagcggaggaactaaacacatcggtaatgcccatagccatcagcacagaggtcaggttgtacttctcctccatcttcatccgggggagatacaccttaatcttgcgctcctccataacgttagagctagtccactccgtcaacttctcgaagttgatgatgctctccagctgctccagtccgctcacttcgtccggaaggagaacgagcatggacatggtacccgaggcaaacgggagctccaggatcttcatcttttcgctcgccatgcttgcgacacggaaaaggccaatctggtacatcatctggacgggcttgctttcctgttcggtcacgcggaagggcatagcctgtgtatcctcgtccttaaaggccttctcccagagacccttgaacacaatggcgttgactagcaccatcgcggtctggctgtcaacactgctaggctggagcacgttgcgaatgataccgttagtctgtgactcaacccaactgttgatgagctcccgggcttggtcagcagccgtctggaagtttatgggctccaggccccctcggtagagttccttcacgcactgcaagtattcgggcagaatagggtagcgctcttctgcgtagagacgagaggccaggctgaaactatacacatcgttgggcttagtgatttggttcagaatatctcgcagactgctgtggacattcacggaggtgccgcactgcgcttcgatcgaatcaccgaatccaggcagcttatcgaatctcacgaccttattgatctgagttcgggtagagtccttggcgcccaggtataccatcgcgagggcggacataatagcaatcggacagtagaaaatattttcgttagcgtggtggaccttcaactccttgaacacgtcgaagcagaattccatggaggccgctccaatagagccgcgcttggaaatcacatttgccaaccctgtgcagacgaggccgctcagggcgagtagagatcggaacgacattgctgaggtgtaatgatgctggggatgaagctcaggctctcagttgcgtctaaaagggaggaagcacttcagcccctcacggaaccccgaccaaccacttcaatttatagccaaacctcatctgcacgaggggaagctgaaggaagaagcacatgcatcaacacatattgccatgcaatatcgcttagcatctagcttgaattcctgcagagacatctcgccacttcaagctcttcagcttgtgaacggacgagacattaggcgacgaatctccgatggtgtgggagaaccagcctatagcggtgtcgggccgggccgggggaaattgcgatgaatccaccagcgctgcagaatggccctgtgccttggagaggtggccaatcgctggcctcactggcctgggatgcaatttctttcaattcccggggattcgccgtccccggtcgtgcgtggtgacttgattggctggtataacgatggtactataactaacgggaagctataacgcaggtccaacgcgtggtatatgcccttcattaatatggctagtactatcatgatccccatatatccttttccgaggccagacacgtccatcattcatctcataaggctaagttcctgaccgtccggacccctccgccaatggcatcagatgtggaacgccgcggtattctgcaggatgaactcatatgtcccgttgcggaaatcatccgtaaggagactggcgtcgttcgctccgaaattcatcaactgccgcgattccttccactgcggccactcaatgtactccccccggttggagttcggatccagcgtatacacaaagctcagatagtacgtgtggctagaactagctgcatagtttggcttgatcccatagaacacctgcagcaggtcacttccgtggaaggtccccagaactggggtgccatagtcataggtcgccaggtacgaccagttcggaagatcaggggagatttcctctgcatacgagaggtctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggc

SEQ ID NO: 44 - DNA, 14851 bp, Artificial Sequence
  BZESCO.23
agtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgctatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggagaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatattatctccgatgaagcctccgttatcccaggttccttgggtttgttgccatctgcgtccttggcctctttgccagacaagaacaccgcatttggtttgtacgaaccatgtcacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaaagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatcccgccagggttttcccagtcacgacgttgtaaaacgacggccagtgaattcgagctcggtacctcgcgaatgcatctagacatgtgtggagagggaggttagtcaagttgggtcagtcaccagacggtgagagagagagagaaagagagagaacttggaggcgagaacggaggggagagagagttgagagtcacagccctattaggcagctccaacatctgcactctgccccagcagaagcagcagtagcagaagcaaatatctcagtcagcaagtcagacggtgtctcagtcggacccagactcttagtaccagctggaagaaggacaagactactactgttacttactccgtactacttttcccacttcccagccccccgctagtgaccttcggatgtgaaccgcggctgttcggcggcgaaacataaaggcgcgcattccgcggaccccaattgctcggctatcacgtgatgcctagtcaatcacctcactttgcgccatcatggcggatgcagcaatcttgatgacttttccatagactaactgctcactagtgaaattagcatcctctacgtagctgctccagtcagcactaatcatcgcgtcattcaggaacactcctcccttttcaatgcctcatgatagtatttctccaaggtcatacaaaagattacagcaacataaggattatatacagctggatttcccacaacattcgaggcaacttttgcgcgcaagccataatgcaaacggtgaaaatggagggagaaaaagaaaaaaaaaaaaaagataaggaggtccaagcccagctagaggtcaatttcagcccaggaccgctgtacaggactgagtggagccattatagaatgctcttcgcggtcctgcagcatgctagtccagggctctctcgtcgggagttgaccgctgcagcttgtctccggggcgcagaccggcacccgctagcgttttagaacctgccagaaacgccatggatatcaagagatcagaaatgggtgggcacttttgcagctttcaaataacaggcatggcaactcatcgctagcgtccgcggcatcacttagaaaccgggaactttccgcttcttcatcagagccttgcgctccttctccttggctgtattgcgaaaaattatattagcattcattcacccaatcagaaaaagaattatagtgcataccttcgtcatagacaaggaactcgtgcagggcacagccagcgggcaccatggggagcacaggcaccttgcgatcagtaaacacttccagaagggcagggccgtcctgctcgatcagccacttgagcttcgcctcaacctccgagggcttggtgcagcggtcggcagcaacacccatggactgggccatgggaacgaagtcggggttcttctggtgagtgtgcgagtaccggtcctcgtagaacaggttctgccattgcgtcaccataccctgttcctcgttgttcaggaggagaaccttcacaccaatgttgaactgagcagcagtggtgagctcagtcagggtcatgttgaacgaggcatcaccatcgatatcaacgacaagagcgtcagggcgggcaaccttggcaccaatggcagcggggagaccgtatcccatagttccaagaccaccagaggtgatcatggtacgggggtggcgccagcggaagtgctgagcagcccacatctgatgctgaccgacaccagtagtgatgacagtgcggtccttcatgtgagcggtgagatcgctgagcttctcaatcacagcctggggcttgatgggtccttcagcagtctgcttctcataaagggagaaggggaagcgagccttccagtcgttgatctgctcgaaccactccttgcgctcagcaacgggctcgacgtggggcaggaggagacggatgttctcagcacagtcaccctcaacagcctcgttggcctggacaaccttgttgatgttcttgggcatgatttcaaagtggacgataccaccacggttctccgaggcagcaagcttggcctggggggcaaacttggagatgttaccagtcacatggtcatcgaagcgagcaccgatagcgatgatgagatcagcctcctgcatggccatgttggcataggcagatccgtgcattcccagcatgtgcagggccttggggtccgtttcgtcgaacccacccagaccctggagagtggtggtgaccggaatgcaagccttgtcggccagctccttcaagatctggggtccttcagggcgagcaaggagaccctgacccacatacaggatgggcttcttggaaacgttcacgaggcgggccacacggttgatggtaccctgcagctgctgcatgctcagttcgcgggcggccatggtcgcagcgctggggagagagggcagggtgctgttcatggggatgggtttacggagaataccggcggtaatatcctcgggcagatcgacgaggacaggtccggggcggccgctggtggcgatttcgaaggcctcctggatgcgacgggggagttcagcgacggacttgaccatgacgttccacttggtgcaagcgcgagagataccgataacatcggcttcctggaagtcatcggtaccgatcaggctggtaggcacctgaccgcagaagacgaccatgggggtaccgtccgagagggcatcctgcatgggggtgataacgttggtggctccagggccggaggtgacaaggacgacaccgggctttccagaggcacgggcgtagccttcggccatgtgtccggcgccctgttcatgtctcggaagaacgaagtcgaagtgtttggagttgtaaatagcatcgaagacgggaagaatggcacctccagggtaaccaactaaaagtagtgggtcaatcttacggtcttgaagaacatcgggaaaccgagaaaaaagacgcgtgactcacagacatgcttgacgccaagacgcagcatcatctcatgaaagatctctcctccactgagtccgacaaaactgtttacgctccaagaaccaggtcagctctactcccatgagtcatctatgttgttgaagggactgcatacgagtcgtccagttcggggacctggcgattttgcagtggcgagacctcattgcggtgaggttcctggttgaaggcagggctggggacaggtcttgtgcctctggaaattcacagacatgttagcaacagtcattgacagcccagtagctatctggatgctaattgtagataatactcactcagccggggcagttgcggtgctctggaatctcttctgagctgaaaagcggtggggcgcggtcgcagcaacggaagcagtggtaaaagtcattctgccggaggcgatatacctctgataatgcagagcgcgcatggcgcttttagaaggtctcataggcatcatcttgatggcggatgactgccaggcctggggaacagagaaaggggagtgctcacaacggggaaaaagaatgggaccaggaagtgtgagtaagagatccgttccttaaggcaggagcgaagactcagacttcggggacagttatggttgcattctagcctgtcctggtctagcgccgttggagtcgaataatctcggtcttcccattggctacggatactccactgctccgatcatcggccttctggacgcgaaagcggtcacctgattgggatgcaagaccctcgtttcgtcctggtctttaccagagcaacgccttgcaaaattgcaataatacaggcgagggatgttgatgatggaaactaacgttgcacaacgacgtatatttctttgctattaattgtcatggtgcggccttttgtgtcaaggttccaactgctatacgatcctgtaatcaactagcccggtttactgctgacatactcctagtcctcatagcaaccaagagacataagggataaaaatcgattacaactcacggactacatcatttagtaattagggcttctataaaatgccactctaaccatacctccatcgatctgtggtaatcacagactttttgagatttctacacatactcctaccgcgggtgccggtccgaatacggctcgctaatgcatctccatcacatgcaggtaaccggcatgaaaagaaagcaagtgttgtatgattttacacccgctactatttactggtgcagtaattctccgtggaggggctgtctatttactatgaagatgactcactcgtaacattggccagtgattcctgaacaatgcctctaattcccaaatcctaggaacataggcacctcggccttaaacttcctttattttcatgaagctgggccgagctcagtggagcatatacatcagggctgcctgcagactgtttgttcggccttgcgtcagatatgcagagtttctgtcgcaatgagaacctcgagggaagttgggtttgcgtgcagagaccgatggctcctcatccagcagtacctgctgctgcacagcgagggcatcgcatcccacgaatagggccaacaaagccggcaagaacatcatgatgggagcgacaattccgtatcaatctcccttgcacgatgttggttgtcactcaccgacactccgtcaccaagatcactattaaaacaagagtgagttcaaggttgcgatcaagatagcgattgcgcagcaacggcacgggataacgccatagctctgatggagccgatcagaccagtaggtaaactagtcagtcagcgttggactggagctgcagagagagttgaacctggacgccgcgcaaaaagcaaagacgcgcctcgtgggcggtggatcaatgatcggatttagtggcagatggcatcacaggcggccaatgaccaccgggccaactggccccgacattccagcaatactgcctaattgactccaccatgcatctcggctattattgaactgggtttgatggatggggaccctcttggaattgtcaaagattttgaagcgaagacgatctattggacggtagagatatactcttgatttagtcgttgggaggcccctggggaaagcaatgatggggaatgttgctgctccactgtggacctcggctatggaattacgtgcttggatctaagatgagctcatggctatgcattgaatgacagtgatatcagcagagcaagcagagaaggatggaatgctaattttctagtgctttgtgcaagggtaaatcagggactgtctgtctggtcttctacacgaaggaaagaccatggctttcacggtgtctgtatttccggatatcctcaattccgtcggtcgattacaatcacatgacttggcttccatttcactactattatgcacacccactacatacatgatcatataaccaattgccctcatccccatcctttaactatagcgaaatggattgattgtctacggcgacacacacctgccgaagaacagaacggcattggttgcgatgtgcttgatgcagaatagaaacgggtggtcggcgcggaactcctcagagacagatgccgcatcgacgccagcttctgccgagccgacgacttcacgaccagcctcgttgatctcagcgtgagcagcgtggacagcttgggaaatcttgagtgactcagccgacgagatcccggacaagttagcggaggaactaaacacatcggtaatgcccatagccatcagcacagaggtcaggttgtacttctcctccatcttcatccgggggagatacaccttaatcttgcgctcctccataacgttagagctagtccactccgtcaacttctcgaagttgatgatgctctccagctgctccagtccgctcacttcgtccggaaggagaacgagcatggacatggtacccgaggcaaacgggagctccaggatcttcatcttttcgctcgccatgcttgcgacacggaaaaggccaatctggtacatcatctggacgggcttgctttcctgttcggtcacgcggaagggcatagcctgtgtatcctcgtccttaaaggccttctcccagagacccttgaacacaatggcgttgactagcaccatcgcggtctggctgtcaacactgctaggctggagcacgttgcgaatgataccgttagtctgtgactcaacccaactgttgatgagctcccgggcttggtcagcagccgtctggaagtttatgggctccaggccccctcggtagagttccttcacgcactgcaagtattcgggcagaatagggtagcgctcttctgcgtagagacgagaggccaggctgaaactatacacatcgttgggcttagtgatttggttcagaatatctcgcagactgctgtggacattcacggaggtgccgcactgcgcttcgatcgaatcaccgaatccaggcagcttatcgaatctcacgaccttattgatctgagttcgggtagagtccttggcgcccaggtataccatcgcgagggcggacataatagcaatcggacagtagaaaatattttcgttagcgtggtggaccttcaactccttgaacacgtcgaagcagaattccatggaggccgctccaatagagcccttgtcatcgtcatcgctagcagtcgcggtggtcttgctggtcgaggtcacgctgccggatccagtgggggtagccgtcgtagtggtgccgccagtagccacgatactcggccacgaggtgacagtcacactgctgtaggtaccaatggcagatgtggccgcacaggtgccgggcacgctgctggcagaggtctcgccccaagaagcaggcacgacggagttacgacggttgttggcggtcagcagagcagcataagaccaggtcaggtcgcgagcggaaagctgctcgccatcagacttgtcgtattgctcggacatggagccgttgcttgcggcgtgagtttcctacttctgttagtacgcaccctctgtcaacatgagcgcttgtctagcgtagacttaccacaatagagacgaagccatcggcgaaagtcttcacggcatctacaatgctactataagtcgaactggacgaagagtaggtgccagtagcagcatcgctgtacagtgccttgaagaagtccagcgacacatctgtgacctccaacgacccctgcttgtcccactggtatagagcatcgtacaactgctctgcggcagccaaggtgcacaggaaccacgggttgccgttgtagtacgtgtcctcagggtaccgacccaccgcaacagcctcgctgtcactgagaccatcgttgagggtatagattgagcggaaagagtctacaacctccttgtggttggcgagcgcgcgcggggagcagggctggaaggtggagtcgtcgcatgcggcctcaggatcaaaggtgtggatgcttcccaggagggtgtttgcgtccttgccggaacggctgctatcgaagttggccagaatgaagctgccggtccagaaggactgcaggtagcagagaatttcgggtgcctgagaatcacaccaggagcaggacgagccgacggccgtcgcgaaggcactaccttcgacaagggcgcggtgttgcacagcaatcgtaaagaaagacgagccattgacttcttcccagagatctgcgatcgcgggttagctctgctggcgcatctttggaaatttaaaataaaacaaacacaccatatcctgtctggttccagtattgagccacatacgacaggtcgttcctaacgaggggccaaacaatgtccgttgcggtgctggtgtagccattgtcctgaccagtcagctacatatgtcacagatcagacgcaagggggtggagaacatacaagcagccactgcccgaagccgatcatagcagttgctctcagagccggaccatctcgctgcggccgtccccaagaaccagtgtaggcagtctcatcgacattgaacttgggttcaccgagaccagcgccgctggacagatcaccagaggggttactgataccctggacaattgcctgggcggagatgtagttctcaatggtggagaggagactggtatctccatttcggaagagatcgacgagggtcttgaggacgagaccagagtcgcgagtccaggtgtagaagtctacaacaaacgacacgccagtcagcatcaatcaatcaatgacacactcatactaaatctgagctcgaaacatacagtccgggttatccgtgctgggactagcaacgacaatgccagagtccgcgcccgacacccaagcaccgtccgccccgatgttattcaggatggcagtacgagccacggtcgcttcgttgctcaaccatgaatccaaggtcgcgcgcttggaaatcacatttgccaaccctgtgcagacgaggccgctcagggcgagtagagatcggaacgacattgctgaggtgtaatgatgctggggatgaagctcaggctctcagttgcgtctaaaagggaggaagcacttcagcccctcacggaaccccgaccaaccacttcaatttatagccaaacctcatctgcacgaggggaagctgaaggaagaagcacatgcatcaacacatattgccatgcaatatcgcttagcatctagcttgaattcctgcagagacatctcgccacttcaagctcttcagcttgtgaacggacgagacattaggcgacgaatctccgatggtgtgggagaaccagcctatagcggtgtcgggccgggccgggggaaattgcgatgaatccaccagcgctgcagaatggccctgtgccttggagaggtggccaatcgctggcctcactggcctgggatgcaatttctttcaattcccggggattcgccgtccccggtcgtgcgtggtgacttgattggctggtataacgatggtactataactaacgggaagctataacgcaggtccaacgcgtggtatatgcccttcattaatatggctagtactatcatgatccccatatatccttttccgaggccagacacgtccatcattcatctcataaggctaagttcctgaccgtccggacccctccgccaatggcatcagatgtggaacgccgcggtattctgcaggatgaactcatatgtcccgttgcggaaatcatccgtaaggagactggcgtcgttcgctccgaaattcatcaactgccgcgattccttccactgcggccactcaatgtactccccccggttggagttcggatccagcgtatacacaaagctcagatagtacgtgtggctagaactagctgcatagtttggcttgatcccatagaacacctgcagcaggtcacttccgtggaaggtccccagaactggggtgccatagtcataggtcgccaggtacgaccagttcggaagatcaggggagatttcctctgcatacgagaggtctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggc

SEQ ID NO: 45 - DNA, 5371 bp, Artificial Sequence
  pCNS43
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacagaagatgatattgaaggagcatttttgggcttggctggagctagtggaggtcaacacatcaatgctattttggtttagtcgtccaggcggatcacaaaatttgtgtcgtttgacaagatggttcatttaggcaactggtcagatcagcccacttgtaagcagtagcggcggcgctcgaagtgtgactcttattagcagacaggaacgaggacattattatcatctgctgcttggtgcacgataacttgtgcgtttgtcaagcaaggtaagtgaacgacccggtcataccttcttaagttcgcccttcctccctttatttcagattcaatctgacttacctattctaccgaagcatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttcaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagccctgggttcgcaaagataattgcatgtttcttccttgaactctcaagcctacaggacacacattcatcgtaggtataaacctcgaaatcattcctactaagatggtatacaatagtaaccatggttgcctagtgaatgctccgtaacacccaatacgccggccgaaacttttttacaactctcctatgagtcgtttacccagaatgcacaggtacacttgtttagaggtaatccttctttctagaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagggacgcatcagcgtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 46 - DNA, 20 bp, Artificial Sequence
  Primer 404
ctcactatagggcgaattgg

SEQ ID NO: 47 - DNA, 20 bp, Artificial Sequence
  Primer 405
ggtgatgtcggcgatatagg

SEQ ID NO: 48 - DNA, 20 bp, Artificial Sequence
  Primer 553
tccaccatgtgaacgagaac

SEQ ID NO: 49 - DNA, 20 bp, Artificial Sequence
  Primer 554
gtggtcagctcggaactcag

SEQ ID NO: 50 - DNA, 20 bp, Artificial Sequence
  Primer 555
tcaaggtccagcacgtgaac

SEQ ID NO: 51 - DNA, 20 bp, Artificial Sequence
  Primer 556
tcggacacgctggtgatctc

SEQ ID NO: 52 - DNA, 20 bp, Artificial Sequence
  Primer 557
tgaaggtccagcacgtgaac

SEQ ID NO: 53 - DNA, 20 bp, Artificial Sequence
  Primer 558
tcggacacgctggtgacttc

SEQ ID NO: 54 - DNA, 20 bp, Artificial Sequence
  Primer 559
tgagctgaaggcccagtacc

SEQ ID NO: 55 - DNA, 20 bp, Artificial Sequence
  Primer 560
gggaagcgctctcatccttg

SEQ ID NO: 56 - DNA, 20 bp, Artificial Sequence
  Primer 561
tgcaccatgccaacgacaac

SEQ ID NO: 57 - DNA, 20 bp, Artificial Sequence
  Primer 562
ttgacgcagaagaggaaagg

SEQ ID NO: 58 - DNA, 20 bp, Artificial Sequence
  Primer 336
gtcctgcgggtaaatagctg

SEQ ID NO: 59 - DNA, 20 bp, Artificial Sequence
  Primer 337
cgtctgctgctccatacaag

SEQ ID NO: 60 - DNA, 20 bp, Artificial Sequence
  Primer 400
gcctcaatcctgggaagaac

SEQ ID NO: 61 - DNA, 20 bp, Artificial Sequence
  Primer 401
gcccattgatgtttcgtagc

SEQ ID NO: 62 - DNA, 5073 bp, Artificial Sequence
  pBZ0026
ggaggagctctgtacagtgaccggtgactctttctggcatgcggagagacggacggtcgcagagaggagggctgagtaataagcgcactcatgtcagctctggcgctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttaacaaactgcgcccacgagttcttccctgacgccgctctcgcgcaggcaagggaactcgatgaatactacgcaaagcacaagcgccccgttggtccactccatggcctccccatctctctcaaagaccagcttcgagtcaagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgaggacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggctttgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacttacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 63 - DNA, 40 bp, Artificial Sequence
  Primer 411
aaaggtctcgggaggagctctgtacagtgaccggtgactc

SEQ ID NO: 64 - DNA, 34 bp, Artificial Sequence
  Primer 415
aaaggtctcaatggacctacgatgaatgtgtgtc

SEQ ID NO: 65 - DNA, 1236 bp, Artificial Sequence
  ostrich ovalbumin cDNA codon optimized glaA (A. niger)
atgtcgttccgatctctactcgccctgagcggcctcgtctgcacagggttggcaaatgtgatttccaagcgcggcagcatttccgctgccaacgctgagttctgcttcgacatgttcaaggagctgaagattcaccacgtcaacgagaacatcttctacagccccctctccattatcagcgctctgagcatggtctacctcggtgctcgcggtaacaccgagagccagatggagaaggtgctgcacttcgacgagattgctggcctcggcggcaccatccacttcaagtgcggcaagtccgtgaacattcacatcctgttcaaggagctgctcagcgatattaccgctccgaccgccaactactccctgcacatcgctaaccgcctctacgccgagaagacccgccccattctgccgatctacctcaagtgcgtcaagaagctgtaccgcgctggcctcgaaaccgtgaacttcaagaccgctagcgaccaggcccgccagctgattaactcctgggtggccaaccagaccaacggccagatccaggacttcctggagcccggcagcgtggatctctccaccgtcctgctcctggtgaacgctgtctacttcaagggcatgtggaagaaggccttccgcgaggaagacacccaggaagtgctcttccgcgtcatggagcaggagagcaagccggtccagatgatgtgccagaacggcaccttcaagatggctgaggtggctgccgagaagattaaggtcctggagctcccctacgctagcggcgagctgtccatgctggtgctcctgcccgaggatgctagctccctggagcagctcgagagcaccatctccttcgagaagctggctaagtggaccagccccaacatgatggagaagaagaccgtgaaggtctacctgccgcgcatgaagctcgaggagaagtacaacctgacctccgtcctgaccgctctgggtatgaccgatctgttctcccccgctgccaacctcagcggcatttccgctgccgaaaccctcaagatgagcgaggctgtgcacgaggccagcatggaggtgagcgaggctggtaccgaggtggctggttccgctggtgctatgggcgagatcaagcacagccccgtgtccgctgagttccgcgctgatcaccccttcctcctgctcattcgccacaacccgaccaacagcatcctgttcttcggccgctacttctccccgtag

SEQ ID NO: 66 - DNA, 1230 bp, Artificial Sequence
  pelican ovalbumin cDNA codon optimized glaA (A. niger)
atgtcgttccgatctctactcgccctgagcggcctcgtctgcacagggttggcaaatgtgatttccaagcgcggctccattggcgctgccagcaccgagttctgcttcgacgtgttcaaggagctgaaggtgcagcacgtcaacgagaacatcttctacagccccctgagcattatctccgctctgagcatggtctacctcggtgctcgagagaacacccgagctcagattgacaaggtggtccacttcgataagatcaccggcttcggcgagcccattgagtcccagtgcggcatctccgtgagcgtccacaccagcctgaaggacatgattacccagatcaccaagccgtccgataactactccctgagcttcgctagccgcctctacgccgaggaaacctaccccattctgccggagtacctccagtgcgtgaaggagctgtacaagggcggcctcgaaaccatttccttccagaccgctgctgaccaggctcgagagctgatcaacagctgggtggagaaccagaccaacggcatgattaagaacattctccagccgggttccgtggatccccagaccgagatggtgctcgtcaacgccgtctacttcaagggcatgtgggagaaggctttcaaggacgaggatacccaggctgtgcccttccgcatgaccgagcaggagagcaagccggtccagatgatgtaccagattggctccttcaaggtggctgtcatggccagcgagaagattaagatcctggagctcccctacgcttccggcgagctgagcatgctcgtgctgctcccggacgatgtcagcggcctggagcagctcgaaaccgctatcaccctggacaagctcaccgagtggaccagctccaacgccatggaggagcgcaagatgaaggtgtacctgccccgcatgaagattgagaagaagtacaacctcacctccgtcctgatcgctctcggcatgaccgatctgttcagctccagcgctaacctcagcggcatctccagcgccgagtccctgaagatgagcgaggctattcacgaggccttcctcgagatctacgaggctggtagcgaggtggtgggtagcaccgaggctggtatggaggtgacctccgtcagcgaggagttccgcgccgatcaccccttcctgttcctcattaagcacaacccgaccaactccatcctgttcttcggccgctgcctcagcccgtag

SEQ ID NO: 67 - DNA, 1230 bp, Artificial Sequence
  pigeon ovalbumin cDNA codon optimized glaA (A. niger)
atgtcgttccgatctctactcgccctgagcggcctcgtctgcacagggttggcaaatgtgatttccaagcgcgctagcattggcgctgccagcgccgagttctccttcgaggtgttcgacgagctgaaggctcagtaccccaaccagaacattatcttcgctccgctgtccatcctcagcgccctgtccatgctgtacctcggtgctcgcggtaacaccaaggctcagattgacaaggtggtccacttcgataagatcaccggcagcggcgaaaccgtcgagtcccagtgctccaccagcgtgaacgtccacacctccctcaaggacaccttcattcagatcaccaagcccagcgataactactccctcagcttcgcttcccgcctgtacgccgaggaaaccttccagattctcccggagtacatccagtgcgtgaaggagctgtacaaggagagcgtggaaaccgtgagcttccagaaggctgccgaccaggctcgcgagctgattaacagctgggccgagtcccagaccaacggcatgattcgcaacattctgcagccgggtagcgtggatccccagaccgagatggtgctggtcaacgctatctacttcaagggcgtgtgggctaaggccttcaaggtcgagggcacccagaccatgcccttccgcgtgaccgagcaggagagcaagccggtccagatgatgtaccagctcggctccttcaaggtgaccgacctgcccccggagaagatgcgcattctggagctcccctacgctagcggcatgctctccatgtgggtcattctgccgtacgatatcagcggcctcgagcagattgagaacgccatcaccttcgagaagctccaggagtggaccagctccaacctgatggaggagaagaccgtgaaggtctacctcccccgcatgaagatggaggagaagtacaacctgaccagcgtgctcatgggcctgggcattaccgacctcttcagctccagcgctgatctgagcggcatctccagcgtggagcgcctgaaggtgtccgaggctgtccaccaggccttcgtcgagattgacgaggctggctccgaggctgctggtattgctggtgctgctgtgaaggatgagtccgctagccccgagttcaaggtcgatcacccgttcctgttcctcattcgccacaaccccaccgatggcatcctgttcctcgcccgctgcgtgagcccgtag

SEQ ID NO: 68 - DNA, 1221 bp, Artificial Sequence
  quail ovalbumin cDNA codon optimized glaA (A. niger)
atgtcgttccgatctctactcgccctgagcggcctcgtctgcacagggttggcaaatgtgatttccaagcgcggcagcattggcgctgcctccatggagttctgcttcgacgtgttcaaggagctgaaggtccaccacgctaacgataacatgctctacagccccttcgctatcctgtccaccctcgccatggtgttcctgggcgctaaggacagcacccgcacccagattaacaaggtggtccacttcgacaagctcccgggcttcggcgatagcatcgaggcccagtgcggcacctccgctaacgtccacagctccctgcgcgacattctcaaccagatcaccaagcagaacgatgcttacagcttctccctggctagccgcctctacgcccaggaaacctacaccgtggtccccgagtacctgcagtgcgtgaaggagctgtaccgcggcggcctcgagtccgtcaacttccagaccgctgctgatcaggctcgaggcctgattaacgcttgggtggagagccagaccaacggcattatccgcaacatcctgcagcccagctccgtggactcccagaccgccatggtgctggtcaacgctattgccttcaagggcctctgggagaaggctttcaaggccgaggatacccagaccatccccttccgcgtgaccgagcaggagagcaagccggtccagatgatgcaccagattggctccttcaaggtggctagcatggcctccgagaagatgaagatcctggagctccccttcgctagcggcaccatgtccatgctggtgctgctcccggacgatgtcagcggcctggagcagctcgagagcaccatttccttcgagaagctcaccgagtggaccagctccagcatcatggaggagcgcaaggtgaaggtctacctgccccgcatgaagatggaggagaagtacaacctcaccagcctgctcatggccatgggcattaccgacctgttctccagctccgctaacctcagcggcattagctccgtgggctccctgaagattccccaggctgtccacgctgcttacgctgagatcaacgaggctggtcgagatgtggtgggtagcgctgaggctggtgtggatgctaccgaggagttccgcgctgatcaccccttcctcttctgcgtgaagcacattgaaaccaacgctatcctgctcttcggccgctgcgtctccccgtag

SEQ ID NO: 69 - DNA, 1230 bp, Artificial Sequence
  plover ovalbumin cDNA codon optimized glaA (A. niger)
atgtcgttccgatctctactcgccctgagcggcctcgtctgcacagggttggcaaatgtgatttccaagcgcggctccattggcgctgccagcaccgagttctgcttcgacgtgttcaaggagctcaaggtgcagcacgtcaacgagaacatcttctacagcccgctgaccattatctccgctctcagcatggtctacctgggtgctcgagagaacacccgagctcagattgacaaggtggtccacttcgataagatccccggcttcggcgataccaccgagtcccagtgcggtacctccgtgagcgtccacaccagcctcaaggacatgttcacccagattaccaagccgtccgataactactccgtgagcttcgctagccgcctgtacgccgaggaaacctaccccatcctcccggagttcctggagtgcgtcaaggagctctacaagggcggcctggagtccattagcttccagaccgctgctgaccaggctcgagagctcatcaactcctgggtggagagccagaccaacggcatgattaagaacatcctccagcccggctccgtcgatagccagaccgagatggtgctggtcaacgctatctacttcaagggcatgtgggagaaggccttcaaggacgaggatacccagaccgtgcccttccgcatgaccgagcaggaaaccaagccggtccagatgatgtaccagattggcaccttcaaggtggctgtcatgccctccgagaagatgaagattctcgagctcccctacgctagcggcgagctgtgcatgctcgtgatgctgcccgacgatgtctccggcctcgaggagctggagagctccattaccgtggagaagctgatggagtggaccagctccaacatgatggaggagcgcaagatgaaggtgttcctcccgcgcatgaagatcgaggagaagtacaacctgaccagcgtcctcatggctctgggcatgaccgacctcttcagctccagcgctaacctgtccggcatttccagcgccgagcccctcaagatgagcgaggctgtgcacgaggccttcattgagatctacgaggctggtagcgaggtggtgggtagcaccggtgctggcatggagattacctccgtcagcgaggagttccgcgccgatcaccccttcctgttcctcattaagcacaacccgaccaactccatcctgttcttcggccgctgcgtgagcccgtag

SEQ ID NO: 70 - DNA, 4978 bp, Artificial Sequence
  pBZ0041
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggcagcatttccgctgccaacgctgagttctgcttcgacatgttcaaggagctgaagattcaccacgtcaacgagaacatcttctacagccccctctccattatcagcgctctgagcatggtctacctcggtgctcgcggtaacaccgagagccagatggagaaggtgctgcacttcgacgagattgctggcctcggcggcaccatccacttcaagtgcggcaagtccgtgaacattcacatcctgttcaaggagctgctcagcgatattaccgctccgaccgccaactactccctgcacatcgctaaccgcctctacgccgagaagacccgccccattctgccgatctacctcaagtgcgtcaagaagctgtaccgcgctggcctcgaaaccgtgaacttcaagaccgctagcgaccaggcccgccagctgattaactcctgggtggccaaccagaccaacggccagatccaggacttcctggagcccggcagcgtggatctctccaccgtcctgctcctggtgaacgctgtctacttcaagggcatgtggaagaaggccttccgcgaggaagacacccaggaagtgctcttccgcgtcatggagcaggagagcaagccggtccagatgatgtgccagaacggcaccttcaagatggctgaggtggctgccgagaagattaaggtcctggagctcccctacgctagcggcgagctgtccatgctggtgctcctgcccgaggatgctagctccctggagcagctcgagagcaccatctccttcgagaagctggctaagtggaccagccccaacatgatggagaagaagaccgtgaaggtctacctgccgcgcatgaagctcgaggagaagtacaacctgacctccgtcctgaccgctctgggtatgaccgatctgttctcccccgctgccaacctcagcggcatttccgctgccgaaaccctcaagatgagcgaggctgtgcacgaggccagcatggaggtgagcgaggctggtaccgaggtggctggttccgctggtgctatgggcgagatcaagcacagccccgtgtccgctgagttccgcgctgatcaccccttcctcctgctcattcgccacaacccgaccaacagcatcctgttcttcggccgctacttctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 71 - DNA, 4972 bp, Artificial Sequence
  pBZ0042
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggctccattggcgctgccagcaccgagttctgcttcgacgtgttcaaggagctcaaggtgcagcacgtcaacgagaacatcttctacagcccgctgaccattatctccgctctcagcatggtctacctgggtgctcgagagaacacccgagctcagattgacaaggtggtccacttcgataagatccccggcttcggcgataccaccgagtcccagtgcggtacctccgtgagcgtccacaccagcctcaaggacatgttcacccagattaccaagccgtccgataactactccgtgagcttcgctagccgcctgtacgccgaggaaacctaccccatcctcccggagttcctggagtgcgtcaaggagctctacaagggcggcctggagtccattagcttccagaccgctgctgaccaggctcgagagctcatcaactcctgggtggagagccagaccaacggcatgattaagaacatcctccagcccggctccgtcgatagccagaccgagatggtgctggtcaacgctatctacttcaagggcatgtgggagaaggccttcaaggacgaggatacccagaccgtgcccttccgcatgaccgagcaggaaaccaagccggtccagatgatgtaccagattggcaccttcaaggtggctgtcatgccctccgagaagatgaagattctcgagctcccctacgctagcggcgagctgtgcatgctcgtgatgctgcccgacgatgtctccggcctcgaggagctggagagctccattaccgtggagaagctgatggagtggaccagctccaacatgatggaggagcgcaagatgaaggtgttcctcccgcgcatgaagatcgaggagaagtacaacctgaccagcgtcctcatggctctgggcatgaccgacctcttcagctccagcgctaacctgtccggcatttccagcgccgagcccctcaagatgagcgaggctgtgcacgaggccttcattgagatctacgaggctggtagcgaggtggtgggtagcaccggtgctggcatggagattacctccgtcagcgaggagttccgcgccgatcaccccttcctgttcctcattaagcacaacccgaccaactccatcctgttcttcggccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 72 - DNA, 4972 bp, Artificial Sequence
  pBZ0043
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggctccattggcgctgccagcaccgagttctgcttcgacgtgttcaaggagctgaaggtgcagcacgtcaacgagaacatcttctacagccccctgagcattatctccgctctgagcatggtctacctcggtgctcgagagaacacccgagctcagattgacaaggtggtccacttcgataagatcaccggcttcggcgagcccattgagtcccagtgcggcatctccgtgagcgtccacaccagcctgaaggacatgattacccagatcaccaagccgtccgataactactccctgagcttcgctagccgcctctacgccgaggaaacctaccccattctgccggagtacctccagtgcgtgaaggagctgtacaagggcggcctcgaaaccatttccttccagaccgctgctgaccaggctcgagagctgatcaacagctgggtggagaaccagaccaacggcatgattaagaacattctccagccgggttccgtggatccccagaccgagatggtgctcgtcaacgccgtctacttcaagggcatgtgggagaaggctttcaaggacgaggatacccaggctgtgcccttccgcatgaccgagcaggagagcaagccggtccagatgatgtaccagattggctccttcaaggtggctgtcatggccagcgagaagattaagatcctggagctcccctacgcttccggcgagctgagcatgctcgtgctgctcccggacgatgtcagcggcctggagcagctcgaaaccgctatcaccctggacaagctcaccgagtggaccagctccaacgccatggaggagcgcaagatgaaggtgtacctgccccgcatgaagattgagaagaagtacaacctcacctccgtcctgatcgctctcggcatgaccgatctgttcagctccagcgctaacctcagcggcatctccagcgccgagtccctgaagatgagcgaggctattcacgaggccttcctcgagatctacgaggctggtagcgaggtggtgggtagcaccgaggctggtatggaggtgacctccgtcagcgaggagttccgcgccgatcaccccttcctgttcctcattaagcacaacccgaccaactccatcctgttcttcggccgctgcctcagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 73 - DNA, 4972 bp, Artificial Sequence
  pBZ0044
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgctagcattggcgctgccagcgccgagttctccttcgaggtgttcgacgagctgaaggctcagtaccccaaccagaacattatcttcgctccgctgtccatcctcagcgccctgtccatgctgtacctcggtgctcgcggtaacaccaaggctcagattgacaaggtggtccacttcgataagatcaccggcagcggcgaaaccgtcgagtcccagtgctccaccagcgtgaacgtccacacctccctcaaggacaccttcattcagatcaccaagcccagcgataactactccctcagcttcgcttcccgcctgtacgccgaggaaaccttccagattctcccggagtacatccagtgcgtgaaggagctgtacaaggagagcgtggaaaccgtgagcttccagaaggctgccgaccaggctcgcgagctgattaacagctgggccgagtcccagaccaacggcatgattcgcaacattctgcagccgggtagcgtggatccccagaccgagatggtgctggtcaacgctatctacttcaagggcgtgtgggctaaggccttcaaggtcgagggcacccagaccatgcccttccgcgtgaccgagcaggagagcaagccggtccagatgatgtaccagctcggctccttcaaggtgaccgacctgcccccggagaagatgcgcattctggagctcccctacgctagcggcatgctctccatgtgggtcattctgccgtacgatatcagcggcctcgagcagattgagaacgccatcaccttcgagaagctccaggagtggaccagctccaacctgatggaggagaagaccgtgaaggtctacctcccccgcatgaagatggaggagaagtacaacctgaccagcgtgctcatgggcctgggcattaccgacctcttcagctccagcgctgatctgagcggcatctccagcgtggagcgcctgaaggtgtccgaggctgtccaccaggccttcgtcgagattgacgaggctggctccgaggctgctggtattgctggtgctgctgtgaaggatgagtccgctagccccgagttcaaggtcgatcacccgttcctgttcctcattcgccacaaccccaccgatggcatcctgttcctcgcccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 74 - DNA, 4963 bp, Artificial Sequence
  pBZ0045
ggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggcagcattggcgctgcctccatggagttctgcttcgacgtgttcaaggagctgaaggtccaccacgctaacgataacatgctctacagccccttcgctatcctgtccaccctcgccatggtgttcctgggcgctaaggacagcacccgcacccagattaacaaggtggtccacttcgacaagctcccgggcttcggcgatagcatcgaggcccagtgcggcacctccgctaacgtccacagctccctgcgcgacattctcaaccagatcaccaagcagaacgatgcttacagcttctccctggctagccgcctctacgcccaggaaacctacaccgtggtccccgagtacctgcagtgcgtgaaggagctgtaccgcggcggcctcgagtccgtcaacttccagaccgctgctgatcaggctcgaggcctgattaacgcttgggtggagagccagaccaacggcattatccgcaacatcctgcagcccagctccgtggactcccagaccgccatggtgctggtcaacgctattgccttcaagggcctctgggagaaggctttcaaggccgaggatacccagaccatccccttccgcgtgaccgagcaggagagcaagccggtccagatgatgcaccagattggctccttcaaggtggctagcatggcctccgagaagatgaagatcctggagctccccttcgctagcggcaccatgtccatgctggtgctgctcccggacgatgtcagcggcctggagcagctcgagagcaccatttccttcgagaagctcaccgagtggaccagctccagcatcatggaggagcgcaaggtgaaggtctacctgccccgcatgaagatggaggagaagtacaacctcaccagcctgctcatggccatgggcattaccgacctgttctccagctccgctaacctcagcggcattagctccgtgggctccctgaagattccccaggctgtccacgctgcttacgctgagatcaacgaggctggtcgagatgtggtgggtagcgctgaggctggtgtggatgctaccgaggagttccgcgctgatcaccccttcctcttctgcgtgaagcacattgaaaccaacgctatcctgctcttcggccgctgcgtctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagccgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtac

SEQ ID NO: 75 - DNA, 50 bp, Artificial Sequence
  primer 728
aataggcgtatcacgaggccctttcgatcattgatccaccgcccacgagg

SEQ ID NO: 76 - DNA, 50 bp, Artificial Sequence
  primer 729
ggagatttcctctgcatacgagaggacggtcaacgctctttccctcagga

SEQ ID NO: 77 - DNA, 22 bp, Artificial Sequence
  primer 733
cctctcgtatgcagaggaaatc

SEQ ID NO: 78 - DNA, 49 bp, Artificial Sequence
  primer 734
attctgtggataaccgtattaccgcatttagtggcagatggcatcacag

SEQ ID NO: 79 - DNA, 2686 bp, Artificial Sequence
  pUC19
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 80 - DNA, 20 bp, Artificial Sequence
  primer 431
gaaagggcctcgtgatacgc

SEQ ID NO: 81 - DNA, 20 bp, Artificial Sequence
  primer 432
gcggtaatacggttatccac

SEQ ID NO: 82 - DNA, 6254 bp, Artificial Sequence
  pBZ0061
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaatgtcgttccgatctctactcgccctgagcggcctcgtctgcacagggttggcaaatgtgatttccaagcgcggctctattggagcggcctccatggaattctgcttcgacgtgttcaaggagttgaaggtccaccacgctaacgaaaatattttctactgtccgattgctattatgtccgccctcgcgatggtatacctgggcgccaaggactctacccgaactcagatcaataaggtcgtgagattcgataagctgcctggattcggtgattcgatcgaagcgcagtgcggcacctccgtgaatgtccacagcagtctgcgagatattctgaaccaaatcactaagcccaacgatgtgtatagtttcagcctggcctctcgtctctacgcagaagagcgctaccctattctgcccgaatacttgcagtgcgtgaaggaactctaccgagggggcctggagcccataaacttccagacggctgctgaccaagcccgggagctcatcaacagttgggttgagtcacagactaacggtatcattcgcaacgtgctccagcctagcagtgttgacagccagaccgcgatggtgctagtcaacgccattgtgttcaagggtctctgggagaaggcctttaaggacgaggatacacaggctatgcccttccgcgtgaccgaacaggaaagcaagcccgtccagatgatgtaccagattggccttttccgtgtcgcaagcatggcgagcgaaaagatgaagatcctggagctcccgtttgcctcgggtaccatgtccatgctcgttctccttccggacgaagtgagcggactggagcagctggagagcatcatcaacttcgagaagttgacggagtggactagctctaacgttatggaggagcgcaagattaaggtgtatctcccccggatgaagatggaggagaagtacaacctgacctctgtgctgatggctatgggcattaccgatgtgtttagttcctccgctaacttgtccgggatctcgtcggctgagtcactcaagatttcccaagctgtccacgctgctcacgctgagatcaacgaggctggtcgtgaagtcgtcggctcggcagaagctggcgtcgatgcggcatctgtctctgaggagttccgcgccgaccacccgtttctattctgcatcaagcacatcgcaaccaatgccgttctgttcttcggcaggtgtgtgtcgccgtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 83 - DNA, 6265 bp, Artificial Sequence
  pBZ0062
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggcagcatttccgctgccaacgctgagttctgcttcgacatgttcaaggagctgaagattcaccacgtcaacgagaacatcttctacagccccctctccattatcagcgctctgagcatggtctacctcggtgctcgcggtaacaccgagagccagatggagaaggtgctgcacttcgacgagattgctggcctcggcggcaccatccacttcaagtgcggcaagtccgtgaacattcacatcctgttcaaggagctgctcagcgatattaccgctccgaccgccaactactccctgcacatcgctaaccgcctctacgccgagaagacccgccccattctgccgatctacctcaagtgcgtcaagaagctgtaccgcgctggcctcgaaaccgtgaacttcaagaccgctagcgaccaggcccgccagctgattaactcctgggtggccaaccagaccaacggccagatccaggacttcctggagcccggcagcgtggatctctccaccgtcctgctcctggtgaacgctgtctacttcaagggcatgtggaagaaggccttccgcgaggaagacacccaggaagtgctcttccgcgtcatggagcaggagagcaagccggtccagatgatgtgccagaacggcaccttcaagatggctgaggtggctgccgagaagattaaggtcctggagctcccctacgctagcggcgagctgtccatgctggtgctcctgcccgaggatgctagctccctggagcagctcgagagcaccatctccttcgagaagctggctaagtggaccagccccaacatgatggagaagaagaccgtgaaggtctacctgccgcgcatgaagctcgaggagaagtacaacctgacctccgtcctgaccgctctgggtatgaccgatctgttctcccccgctgccaacctcagcggcatttccgctgccgaaaccctcaagatgagcgaggctgtgcacgaggccagcatggaggtgagcgaggctggtaccgaggtggctggttccgctggtgctatgggcgagatcaagcacagccccgtgtccgctgagttccgcgctgatcaccccttcctcctgctcattcgccacaacccgaccaacagcatcctgttcttcggccgctacttctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 84 - DNA, 6259 bp, Artificial Sequence
  pBZ0063
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggctccattggcgctgccagcaccgagttctgcttcgacgtgttcaaggagctcaaggtgcagcacgtcaacgagaacatcttctacagcccgctgaccattatctccgctctcagcatggtctacctgggtgctcgagagaacacccgagctcagattgacaaggtggtccacttcgataagatccccggcttcggcgataccaccgagtcccagtgcggtacctccgtgagcgtccacaccagcctcaaggacatgttcacccagattaccaagccgtccgataactactccgtgagcttcgctagccgcctgtacgccgaggaaacctaccccatcctcccggagttcctggagtgcgtcaaggagctctacaagggcggcctggagtccattagcttccagaccgctgctgaccaggctcgagagctcatcaactcctgggtggagagccagaccaacggcatgattaagaacatcctccagcccggctccgtcgatagccagaccgagatggtgctggtcaacgctatctacttcaagggcatgtgggagaaggccttcaaggacgaggatacccagaccgtgcccttccgcatgaccgagcaggaaaccaagccggtccagatgatgtaccagattggcaccttcaaggtggctgtcatgccctccgagaagatgaagattctcgagctcccctacgctagcggcgagctgtgcatgctcgtgatgctgcccgacgatgtctccggcctcgaggagctggagagctccattaccgtggagaagctgatggagtggaccagctccaacatgatggaggagcgcaagatgaaggtgttcctcccgcgcatgaagatcgaggagaagtacaacctgaccagcgtcctcatggctctgggcatgaccgacctcttcagctccagcgctaacctgtccggcatttccagcgccgagcccctcaagatgagcgaggctgtgcacgaggccttcattgagatctacgaggctggtagcgaggtggtgggtagcaccggtgctggcatggagattacctccgtcagcgaggagttccgcgccgatcaccccttcctgttcctcattaagcacaacccgaccaactccatcctgttcttcggccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 85 - DNA, 6259 bp, Artificial Sequence
  pBZ0064
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggctccattggcgctgccagcaccgagttctgcttcgacgtgttcaaggagctgaaggtgcagcacgtcaacgagaacatcttctacagccccctgagcattatctccgctctgagcatggtctacctcggtgctcgagagaacacccgagctcagattgacaaggtggtccacttcgataagatcaccggcttcggcgagcccattgagtcccagtgcggcatctccgtgagcgtccacaccagcctgaaggacatgattacccagatcaccaagccgtccgataactactccctgagcttcgctagccgcctctacgccgaggaaacctaccccattctgccggagtacctccagtgcgtgaaggagctgtacaagggcggcctcgaaaccatttccttccagaccgctgctgaccaggctcgagagctgatcaacagctgggtggagaaccagaccaacggcatgattaagaacattctccagccgggttccgtggatccccagaccgagatggtgctcgtcaacgccgtctacttcaagggcatgtgggagaaggctttcaaggacgaggatacccaggctgtgcccttccgcatgaccgagcaggagagcaagccggtccagatgatgtaccagattggctccttcaaggtggctgtcatggccagcgagaagattaagatcctggagctcccctacgcttccggcgagctgagcatgctcgtgctgctcccggacgatgtcagcggcctggagcagctcgaaaccgctatcaccctggacaagctcaccgagtggaccagctccaacgccatggaggagcgcaagatgaaggtgtacctgccccgcatgaagattgagaagaagtacaacctcacctccgtcctgatcgctctcggcatgaccgatctgttcagctccagcgctaacctcagcggcatctccagcgccgagtccctgaagatgagcgaggctattcacgaggccttcctcgagatctacgaggctggtagcgaggtggtgggtagcaccgaggctggtatggaggtgacctccgtcagcgaggagttccgcgccgatcaccccttcctgttcctcattaagcacaacccgaccaactccatcctgttcttcggccgctgcctcagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 86 - DNA, 6259 bp, Artificial Sequence
  pBZ0065
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgctagcattggcgctgccagcgccgagttctccttcgaggtgttcgacgagctgaaggctcagtaccccaaccagaacattatcttcgctccgctgtccatcctcagcgccctgtccatgctgtacctcggtgctcgcggtaacaccaaggctcagattgacaaggtggtccacttcgataagatcaccggcagcggcgaaaccgtcgagtcccagtgctccaccagcgtgaacgtccacacctccctcaaggacaccttcattcagatcaccaagcccagcgataactactccctcagcttcgcttcccgcctgtacgccgaggaaaccttccagattctcccggagtacatccagtgcgtgaaggagctgtacaaggagagcgtggaaaccgtgagcttccagaaggctgccgaccaggctcgcgagctgattaacagctgggccgagtcccagaccaacggcatgattcgcaacattctgcagccgggtagcgtggatccccagaccgagatggtgctggtcaacgctatctacttcaagggcgtgtgggctaaggccttcaaggtcgagggcacccagaccatgcccttccgcgtgaccgagcaggagagcaagccggtccagatgatgtaccagctcggctccttcaaggtgaccgacctgcccccggagaagatgcgcattctggagctcccctacgctagcggcatgctctccatgtgggtcattctgccgtacgatatcagcggcctcgagcagattgagaacgccatcaccttcgagaagctccaggagtggaccagctccaacctgatggaggagaagaccgtgaaggtctacctcccccgcatgaagatggaggagaagtacaacctgaccagcgtgctcatgggcctgggcattaccgacctcttcagctccagcgctgatctgagcggcatctccagcgtggagcgcctgaaggtgtccgaggctgtccaccaggccttcgtcgagattgacgaggctggctccgaggctgctggtattgctggtgctgctgtgaaggatgagtccgctagccccgagttcaaggtcgatcacccgttcctgttcctcattcgccacaaccccaccgatggcatcctgttcctcgcccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 87 - DNA, 6250 bp, Artificial Sequence
  pBZ0066
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcggcagcattggcgctgcctccatggagttctgcttcgacgtgttcaaggagctgaaggtccaccacgctaacgataacatgctctacagccccttcgctatcctgtccaccctcgccatggtgttcctgggcgctaaggacagcacccgcacccagattaacaaggtggtccacttcgacaagctcccgggcttcggcgatagcatcgaggcccagtgcggcacctccgctaacgtccacagctccctgcgcgacattctcaaccagatcaccaagcagaacgatgcttacagcttctccctggctagccgcctctacgcccaggaaacctacaccgtggtccccgagtacctgcagtgcgtgaaggagctgtaccgcggcggcctcgagtccgtcaacttccagaccgctgctgatcaggctcgaggcctgattaacgcttgggtggagagccagaccaacggcattatccgcaacatcctgcagcccagctccgtggactcccagaccgccatggtgctggtcaacgctattgccttcaagggcctctgggagaaggctttcaaggccgaggatacccagaccatccccttccgcgtgaccgagcaggagagcaagccggtccagatgatgcaccagattggctccttcaaggtggctagcatggcctccgagaagatgaagatcctggagctccccttcgctagcggcaccatgtccatgctggtgctgctcccggacgatgtcagcggcctggagcagctcgagagcaccatttccttcgagaagctcaccgagtggaccagctccagcatcatggaggagcgcaaggtgaaggtctacctgccccgcatgaagatggaggagaagtacaacctcaccagcctgctcatggccatgggcattaccgacctgttctccagctccgctaacctcagcggcattagctccgtgggctccctgaagattccccaggctgtccacgctgcttacgctgagatcaacgaggctggtcgagatgtggtgggtagcgctgaggctggtgtggatgctaccgaggagttccgcgctgatcaccccttcctcttctgcgtgaagcacattgaaaccaacgctatcctgctcttcggccgctgcgtctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 88 - protein, 56 aa, Artificial Sequence
  protein sequence GLA54
MSFRSLLALSGLVCTGLANVISKRATLDSWLSNEATVARTAILNNIGADGAWVSKR

SEQ ID NO: 89 - DNA, 168 bp, Artificial Sequence
  DNA sequence GLA54
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgaagcgc

SEQ ID NO: 90 - protein, 102 aa, Artificial Sequence
  protein sequence GLA100
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSKR

SEQ ID NO: 91 - DNA, 381 bp, Artificial Sequence
  DNA sequence GLA100
atgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaagcgc

SEQ ID NO: 92 - DNA, 26 bp, Artificial Sequence
  primer 715
aaaggtctctgcgcggcagcatttcc

SEQ ID NO: 93 - DNA, 39 bp, Artificial Sequence
  primer 422
aaaggtctctaagcctaggggctgaagtagcggccgaag

SEQ ID NO: 94 - DNA, 31 bp, Artificial Sequence
  primer 716
aaaggtctctgcgcggctccattggcgctgc

SEQ ID NO: 95 - DNA, 39 bp, Artificial Sequence
  primer 424
aaaggtctctaagcctaaggggagacgcagcggccgaag

SEQ ID NO: 96 - DNA, 37 bp, Artificial Sequence
  primer 717
aaaggtctctgcgcggctccattggcgctgccagcac

SEQ ID NO: 97 - DNA, 39 bp, Artificial Sequence
  primer 426
aaaggtctctaagcctaaggggagaggcagcggccgaag

SEQ ID NO: 98 - DNA, 26 bp, Artificial Sequence
  primer 718
aaaggtctctgcgcgctagcattggc

SEQ ID NO: 99 - DNA, 34 bp, Artificial Sequence
  primer 428
aaaggtctctaagcctaaggggagacgcagcggg

SEQ ID NO: 100 - DNA, 26 bp, Artificial Sequence
  primer 719
aaaggtctctgcgcggcagcattggc

SEQ ID NO: 101 - DNA, 33 bp, Artificial Sequence
  primer 430
aaaggtctctaagcctacgggctcacgcagcgg

SEQ ID NO: 102 - DNA, 5074 bp, Artificial Sequence
  pBZ0046
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgaagcgcggcagcatttccgctgccaacgctgagttctgcttcgacatgttcaaggagctgaagattcaccacgtcaacgagaacatcttctacagccccctctccattatcagcgctctgagcatggtctacctcggtgctcgcggtaacaccgagagccagatggagaaggtgctgcacttcgacgagattgctggcctcggcggcaccatccacttcaagtgcggcaagtccgtgaacattcacatcctgttcaaggagctgctcagcgatattaccgctccgaccgccaactactccctgcacatcgctaaccgcctctacgccgagaagacccgccccattctgccgatctacctcaagtgcgtcaagaagctgtaccgcgctggcctcgaaaccgtgaacttcaagaccgctagcgaccaggcccgccagctgattaactcctgggtggccaaccagaccaacggccagatccaggacttcctggagcccggcagcgtggatctctccaccgtcctgctcctggtgaacgctgtctacttcaagggcatgtggaagaaggccttccgcgaggaagacacccaggaagtgctcttccgcgtcatggagcaggagagcaagccggtccagatgatgtgccagaacggcaccttcaagatggctgaggtggctgccgagaagattaaggtcctggagctcccctacgctagcggcgagctgtccatgctggtgctcctgcccgaggatgctagctccctggagcagctcgagagcaccatctccttcgagaagctggctaagtggaccagccccaacatgatggagaagaagaccgtgaaggtctacctgccgcgcatgaagctcgaggagaagtacaacctgacctccgtcctgaccgctctgggtatgaccgatctgttctcccccgctgccaacctcagcggcatttccgctgccgaaaccctcaagatgagcgaggctgtgcacgaggccagcatggaggtgagcgaggctggtaccgaggtggctggttccgctggtgctatgggcgagatcaagcacagccccgtgtccgctgagttccgcgctgatcaccccttcctcctgctcattcgccacaacccgaccaacagcatcctgttcttcggccgctacttctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 103 - DNA, 5068 bp, Artificial Sequence
  pBZ0047
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgaagcgcggctccattggcgctgccagcaccgagttctgcttcgacgtgttcaaggagctcaaggtgcagcacgtcaacgagaacatcttctacagcccgctgaccattatctccgctctcagcatggtctacctgggtgctcgagagaacacccgagctcagattgacaaggtggtccacttcgataagatccccggcttcggcgataccaccgagtcccagtgcggtacctccgtgagcgtccacaccagcctcaaggacatgttcacccagattaccaagccgtccgataactactccgtgagcttcgctagccgcctgtacgccgaggaaacctaccccatcctcccggagttcctggagtgcgtcaaggagctctacaagggcggcctggagtccattagcttccagaccgctgctgaccaggctcgagagctcatcaactcctgggtggagagccagaccaacggcatgattaagaacatcctccagcccggctccgtcgatagccagaccgagatggtgctggtcaacgctatctacttcaagggcatgtgggagaaggccttcaaggacgaggatacccagaccgtgcccttccgcatgaccgagcaggaaaccaagccggtccagatgatgtaccagattggcaccttcaaggtggctgtcatgccctccgagaagatgaagattctcgagctcccctacgctagcggcgagctgtgcatgctcgtgatgctgcccgacgatgtctccggcctcgaggagctggagagctccattaccgtggagaagctgatggagtggaccagctccaacatgatggaggagcgcaagatgaaggtgttcctcccgcgcatgaagatcgaggagaagtacaacctgaccagcgtcctcatggctctgggcatgaccgacctcttcagctccagcgctaacctgtccggcatttccagcgccgagcccctcaagatgagcgaggctgtgcacgaggccttcattgagatctacgaggctggtagcgaggtggtgggtagcaccggtgctggcatggagattacctccgtcagcgaggagttccgcgccgatcaccccttcctgttcctcattaagcacaacccgaccaactccatcctgttcttcggccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 104 - DNA, 5068 bp, Artificial Sequence
  pBZ0048
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgaagcgcggctccattggcgctgccagcaccgagttctgcttcgacgtgttcaaggagctgaaggtgcagcacgtcaacgagaacatcttctacagccccctgagcattatctccgctctgagcatggtctacctcggtgctcgagagaacacccgagctcagattgacaaggtggtccacttcgataagatcaccggcttcggcgagcccattgagtcccagtgcggcatctccgtgagcgtccacaccagcctgaaggacatgattacccagatcaccaagccgtccgataactactccctgagcttcgctagccgcctctacgccgaggaaacctaccccattctgccggagtacctccagtgcgtgaaggagctgtacaagggcggcctcgaaaccatttccttccagaccgctgctgaccaggctcgagagctgatcaacagctgggtggagaaccagaccaacggcatgattaagaacattctccagccgggttccgtggatccccagaccgagatggtgctcgtcaacgccgtctacttcaagggcatgtgggagaaggctttcaaggacgaggatacccaggctgtgcccttccgcatgaccgagcaggagagcaagccggtccagatgatgtaccagattggctccttcaaggtggctgtcatggccagcgagaagattaagatcctggagctcccctacgcttccggcgagctgagcatgctcgtgctgctcccggacgatgtcagcggcctggagcagctcgaaaccgctatcaccctggacaagctcaccgagtggaccagctccaacgccatggaggagcgcaagatgaaggtgtacctgccccgcatgaagattgagaagaagtacaacctcacctccgtcctgatcgctctcggcatgaccgatctgttcagctccagcgctaacctcagcggcatctccagcgccgagtccctgaagatgagcgaggctattcacgaggccttcctcgagatctacgaggctggtagcgaggtggtgggtagcaccgaggctggtatggaggtgacctccgtcagcgaggagttccgcgccgatcaccccttcctgttcctcattaagcacaacccgaccaactccatcctgttcttcggccgctgcctcagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 105 - DNA, 5068 bp, Artificial Sequence
  pBZ0049
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgaagcgcgctagcattggcgctgccagcgccgagttctccttcgaggtgttcgacgagctgaaggctcagtaccccaaccagaacattatcttcgctccgctgtccatcctcagcgccctgtccatgctgtacctcggtgctcgcggtaacaccaaggctcagattgacaaggtggtccacttcgataagatcaccggcagcggcgaaaccgtcgagtcccagtgctccaccagcgtgaacgtccacacctccctcaaggacaccttcattcagatcaccaagcccagcgataactactccctcagcttcgcttcccgcctgtacgccgaggaaaccttccagattctcccggagtacatccagtgcgtgaaggagctgtacaaggagagcgtggaaaccgtgagcttccagaaggctgccgaccaggctcgcgagctgattaacagctgggccgagtcccagaccaacggcatgattcgcaacattctgcagccgggtagcgtggatccccagaccgagatggtgctggtcaacgctatctacttcaagggcgtgtgggctaaggccttcaaggtcgagggcacccagaccatgcccttccgcgtgaccgagcaggagagcaagccggtccagatgatgtaccagctcggctccttcaaggtgaccgacctgcccccggagaagatgcgcattctggagctcccctacgctagcggcatgctctccatgtgggtcattctgccgtacgatatcagcggcctcgagcagattgagaacgccatcaccttcgagaagctccaggagtggaccagctccaacctgatggaggagaagaccgtgaaggtctacctcccccgcatgaagatggaggagaagtacaacctgaccagcgtgctcatgggcctgggcattaccgacctcttcagctccagcgctgatctgagcggcatctccagcgtggagcgcctgaaggtgtccgaggctgtccaccaggccttcgtcgagattgacgaggctggctccgaggctgctggtattgctggtgctgctgtgaaggatgagtccgctagccccgagttcaaggtcgatcacccgttcctgttcctcattcgccacaaccccaccgatggcatcctgttcctcgcccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 106 - DNA, 5059 bp, Artificial Sequence
  pBZ0050
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgaagcgcggcagcattggcgctgcctccatggagttctgcttcgacgtgttcaaggagctgaaggtccaccacgctaacgataacatgctctacagccccttcgctatcctgtccaccctcgccatggtgttcctgggcgctaaggacagcacccgcacccagattaacaaggtggtccacttcgacaagctcccgggcttcggcgatagcatcgaggcccagtgcggcacctccgctaacgtccacagctccctgcgcgacattctcaaccagatcaccaagcagaacgatgcttacagcttctccctggctagccgcctctacgcccaggaaacctacaccgtggtccccgagtacctgcagtgcgtgaaggagctgtaccgcggcggcctcgagtccgtcaacttccagaccgctgctgatcaggctcgaggcctgattaacgcttgggtggagagccagaccaacggcattatccgcaacatcctgcagcccagctccgtggactcccagaccgccatggtgctggtcaacgctattgccttcaagggcctctgggagaaggctttcaaggccgaggatacccagaccatccccttccgcgtgaccgagcaggagagcaagccggtccagatgatgcaccagattggctccttcaaggtggctagcatggcctccgagaagatgaagatcctggagctccccttcgctagcggcaccatgtccatgctggtgctgctcccggacgatgtcagcggcctggagcagctcgagagcaccatttccttcgagaagctcaccgagtggaccagctccagcatcatggaggagcgcaaggtgaaggtctacctgccccgcatgaagatggaggagaagtacaacctcaccagcctgctcatggccatgggcattaccgacctgttctccagctccgctaacctcagcggcattagctccgtgggctccctgaagattccccaggctgtccacgctgcttacgctgagatcaacgaggctggtcgagatgtggtgggtagcgctgaggctggtgtggatgctaccgaggagttccgcgctgatcaccccttcctcttctgcgtgaagcacattgaaaccaacgctatcctgctcttcggccgctgcgtctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 107 - DNA, 5287 bp, Artificial Sequence
  pBZ0051
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaagcgcggcagcatttccgctgccaacgctgagttctgcttcgacatgttcaaggagctgaagattcaccacgtcaacgagaacatcttctacagccccctctccattatcagcgctctgagcatggtctacctcggtgctcgcggtaacaccgagagccagatggagaaggtgctgcacttcgacgagattgctggcctcggcggcaccatccacttcaagtgcggcaagtccgtgaacattcacatcctgttcaaggagctgctcagcgatattaccgctccgaccgccaactactccctgcacatcgctaaccgcctctacgccgagaagacccgccccattctgccgatctacctcaagtgcgtcaagaagctgtaccgcgctggcctcgaaaccgtgaacttcaagaccgctagcgaccaggcccgccagctgattaactcctgggtggccaaccagaccaacggccagatccaggacttcctggagcccggcagcgtggatctctccaccgtcctgctcctggtgaacgctgtctacttcaagggcatgtggaagaaggccttccgcgaggaagacacccaggaagtgctcttccgcgtcatggagcaggagagcaagccggtccagatgatgtgccagaacggcaccttcaagatggctgaggtggctgccgagaagattaaggtcctggagctcccctacgctagcggcgagctgtccatgctggtgctcctgcccgaggatgctagctccctggagcagctcgagagcaccatctccttcgagaagctggctaagtggaccagccccaacatgatggagaagaagaccgtgaaggtctacctgccgcgcatgaagctcgaggagaagtacaacctgacctccgtcctgaccgctctgggtatgaccgatctgttctcccccgctgccaacctcagcggcatttccgctgccgaaaccctcaagatgagcgaggctgtgcacgaggccagcatggaggtgagcgaggctggtaccgaggtggctggttccgctggtgctatgggcgagatcaagcacagccccgtgtccgctgagttccgcgctgatcaccccttcctcctgctcattcgccacaacccgaccaacagcatcctgttcttcggccgctacttctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 108 - DNA, 5281 bp, Artificial Sequence
  pBZ0052
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaagcgcggctccattggcgctgccagcaccgagttctgcttcgacgtgttcaaggagctcaaggtgcagcacgtcaacgagaacatcttctacagcccgctgaccattatctccgctctcagcatggtctacctgggtgctcgagagaacacccgagctcagattgacaaggtggtccacttcgataagatccccggcttcggcgataccaccgagtcccagtgcggtacctccgtgagcgtccacaccagcctcaaggacatgttcacccagattaccaagccgtccgataactactccgtgagcttcgctagccgcctgtacgccgaggaaacctaccccatcctcccggagttcctggagtgcgtcaaggagctctacaagggcggcctggagtccattagcttccagaccgctgctgaccaggctcgagagctcatcaactcctgggtggagagccagaccaacggcatgattaagaacatcctccagcccggctccgtcgatagccagaccgagatggtgctggtcaacgctatctacttcaagggcatgtgggagaaggccttcaaggacgaggatacccagaccgtgcccttccgcatgaccgagcaggaaaccaagccggtccagatgatgtaccagattggcaccttcaaggtggctgtcatgccctccgagaagatgaagattctcgagctcccctacgctagcggcgagctgtgcatgctcgtgatgctgcccgacgatgtctccggcctcgaggagctggagagctccattaccgtggagaagctgatggagtggaccagctccaacatgatggaggagcgcaagatgaaggtgttcctcccgcgcatgaagatcgaggagaagtacaacctgaccagcgtcctcatggctctgggcatgaccgacctcttcagctccagcgctaacctgtccggcatttccagcgccgagcccctcaagatgagcgaggctgtgcacgaggccttcattgagatctacgaggctggtagcgaggtggtgggtagcaccggtgctggcatggagattacctccgtcagcgaggagttccgcgccgatcaccccttcctgttcctcattaagcacaacccgaccaactccatcctgttcttcggccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 109 - DNA, 5281 bp, Artificial Sequence
  pBZ0053
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaagcgcggctccattggcgctgccagcaccgagttctgcttcgacgtgttcaaggagctgaaggtgcagcacgtcaacgagaacatcttctacagccccctgagcattatctccgctctgagcatggtctacctcggtgctcgagagaacacccgagctcagattgacaaggtggtccacttcgataagatcaccggcttcggcgagcccattgagtcccagtgcggcatctccgtgagcgtccacaccagcctgaaggacatgattacccagatcaccaagccgtccgataactactccctgagcttcgctagccgcctctacgccgaggaaacctaccccattctgccggagtacctccagtgcgtgaaggagctgtacaagggcggcctcgaaaccatttccttccagaccgctgctgaccaggctcgagagctgatcaacagctgggtggagaaccagaccaacggcatgattaagaacattctccagccgggttccgtggatccccagaccgagatggtgctcgtcaacgccgtctacttcaagggcatgtgggagaaggctttcaaggacgaggatacccaggctgtgcccttccgcatgaccgagcaggagagcaagccggtccagatgatgtaccagattggctccttcaaggtggctgtcatggccagcgagaagattaagatcctggagctcccctacgcttccggcgagctgagcatgctcgtgctgctcccggacgatgtcagcggcctggagcagctcgaaaccgctatcaccctggacaagctcaccgagtggaccagctccaacgccatggaggagcgcaagatgaaggtgtacctgccccgcatgaagattgagaagaagtacaacctcacctccgtcctgatcgctctcggcatgaccgatctgttcagctccagcgctaacctcagcggcatctccagcgccgagtccctgaagatgagcgaggctattcacgaggccttcctcgagatctacgaggctggtagcgaggtggtgggtagcaccgaggctggtatggaggtgacctccgtcagcgaggagttccgcgccgatcaccccttcctgttcctcattaagcacaacccgaccaactccatcctgttcttcggccgctgcctcagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 110 - DNA, 5281 bp, Artificial Sequence
  pBZ0054
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaagcgcgctagcattggcgctgccagcgccgagttctccttcgaggtgttcgacgagctgaaggctcagtaccccaaccagaacattatcttcgctccgctgtccatcctcagcgccctgtccatgctgtacctcggtgctcgcggtaacaccaaggctcagattgacaaggtggtccacttcgataagatcaccggcagcggcgaaaccgtcgagtcccagtgctccaccagcgtgaacgtccacacctccctcaaggacaccttcattcagatcaccaagcccagcgataactactccctcagcttcgcttcccgcctgtacgccgaggaaaccttccagattctcccggagtacatccagtgcgtgaaggagctgtacaaggagagcgtggaaaccgtgagcttccagaaggctgccgaccaggctcgcgagctgattaacagctgggccgagtcccagaccaacggcatgattcgcaacattctgcagccgggtagcgtggatccccagaccgagatggtgctggtcaacgctatctacttcaagggcgtgtgggctaaggccttcaaggtcgagggcacccagaccatgcccttccgcgtgaccgagcaggagagcaagccggtccagatgatgtaccagctcggctccttcaaggtgaccgacctgcccccggagaagatgcgcattctggagctcccctacgctagcggcatgctctccatgtgggtcattctgccgtacgatatcagcggcctcgagcagattgagaacgccatcaccttcgagaagctccaggagtggaccagctccaacctgatggaggagaagaccgtgaaggtctacctcccccgcatgaagatggaggagaagtacaacctgaccagcgtgctcatgggcctgggcattaccgacctcttcagctccagcgctgatctgagcggcatctccagcgtggagcgcctgaaggtgtccgaggctgtccaccaggccttcgtcgagattgacgaggctggctccgaggctgctggtattgctggtgctgctgtgaaggatgagtccgctagccccgagttcaaggtcgatcacccgttcctgttcctcattcgccacaaccccaccgatggcatcctgttcctcgcccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 111 - DNA, 5272 bp, Artificial Sequence
  pBZ0055
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaagcgcggcagcattggcgctgcctccatggagttctgcttcgacgtgttcaaggagctgaaggtccaccacgctaacgataacatgctctacagccccttcgctatcctgtccaccctcgccatggtgttcctgggcgctaaggacagcacccgcacccagattaacaaggtggtccacttcgacaagctcccgggcttcggcgatagcatcgaggcccagtgcggcacctccgctaacgtccacagctccctgcgcgacattctcaaccagatcaccaagcagaacgatgcttacagcttctccctggctagccgcctctacgcccaggaaacctacaccgtggtccccgagtacctgcagtgcgtgaaggagctgtaccgcggcggcctcgagtccgtcaacttccagaccgctgctgatcaggctcgaggcctgattaacgcttgggtggagagccagaccaacggcattatccgcaacatcctgcagcccagctccgtggactcccagaccgccatggtgctggtcaacgctattgccttcaagggcctctgggagaaggctttcaaggccgaggatacccagaccatccccttccgcgtgaccgagcaggagagcaagccggtccagatgatgcaccagattggctccttcaaggtggctagcatggcctccgagaagatgaagatcctggagctccccttcgctagcggcaccatgtccatgctggtgctgctcccggacgatgtcagcggcctggagcagctcgagagcaccatttccttcgagaagctcaccgagtggaccagctccagcatcatggaggagcgcaaggtgaaggtctacctgccccgcatgaagatggaggagaagtacaacctcaccagcctgctcatggccatgggcattaccgacctgttctccagctccgctaacctcagcggcattagctccgtgggctccctgaagattccccaggctgtccacgctgcttacgctgagatcaacgaggctggtcgagatgtggtgggtagcgctgaggctggtgtggatgctaccgaggagttccgcgctgatcaccccttcctcttctgcgtgaagcacattgaaaccaacgctatcctgctcttcggccgctgcgtctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 112 - DNA, 6688 bp, Artificial Sequence
  pBZ0056
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaccattgagaactacatctccgcccaggcaattgtccagggtatcagtaacccctctggtgatctgtccagcggcgctggcctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggcctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacttctgccagcagcgtgcccggcacctgtgcggccacatctgccattggtacctacagcagtgtgactgtcacctcgtggccgagtatcgtggctactggcggcaccactacgacggcttccaagtctgtcatctccaagcgcggcagcatttccgctgccaacgctgagttctgcttcgacatgttcaaggagctgaagattcaccacgtcaacgagaacatcttctacagccccctctccattatcagcgctctgagcatggtctacctcggtgctcgcggtaacaccgagagccagatggagaaggtgctgcacttcgacgagattgctggcctcggcggcaccatccacttcaagtgcggcaagtccgtgaacattcacatcctgttcaaggagctgctcagcgatattaccgctccgaccgccaactactccctgcacatcgctaaccgcctctacgccgagaagacccgccccattctgccgatctacctcaagtgcgtcaagaagctgtaccgcgctggcctcgaaaccgtgaacttcaagaccgctagcgaccaggcccgccagctgattaactcctgggtggccaaccagaccaacggccagatccaggacttcctggagcccggcagcgtggatctctccaccgtcctgctcctggtgaacgctgtctacttcaagggcatgtggaagaaggccttccgcgaggaagacacccaggaagtgctcttccgcgtcatggagcaggagagcaagccggtccagatgatgtgccagaacggcaccttcaagatggctgaggtggctgccgagaagattaaggtcctggagctcccctacgctagcggcgagctgtccatgctggtgctcctgcccgaggatgctagctccctggagcagctcgagagcaccatctccttcgagaagctggctaagtggaccagccccaacatgatggagaagaagaccgtgaaggtctacctgccgcgcatgaagctcgaggagaagtacaacctgacctccgtcctgaccgctctgggtatgaccgatctgttctcccccgctgccaacctcagcggcatttccgctgccgaaaccctcaagatgagcgaggctgtgcacgaggccagcatggaggtgagcgaggctggtaccgaggtggctggttccgctggtgctatgggcgagatcaagcacagccccgtgtccgctgagttccgcgctgatcaccccttcctcctgctcattcgccacaacccgaccaacagcatcctgttcttcggccgctacttctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 113 - DNA, 6682 bp, Artificial Sequence
  pBZ0057
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaccattgagaactacatctccgcccaggcaattgtccagggtatcagtaacccctctggtgatctgtccagcggcgctggcctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggcctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacttctgccagcagcgtgcccggcacctgtgcggccacatctgccattggtacctacagcagtgtgactgtcacctcgtggccgagtatcgtggctactggcggcaccactacgacggcttccaagtctgtcatctccaagcgcggctccattggcgctgccagcaccgagttctgcttcgacgtgttcaaggagctcaaggtgcagcacgtcaacgagaacatcttctacagcccgctgaccattatctccgctctcagcatggtctacctgggtgctcgagagaacacccgagctcagattgacaaggtggtccacttcgataagatccccggcttcggcgataccaccgagtcccagtgcggtacctccgtgagcgtccacaccagcctcaaggacatgttcacccagattaccaagccgtccgataactactccgtgagcttcgctagccgcctgtacgccgaggaaacctaccccatcctcccggagttcctggagtgcgtcaaggagctctacaagggcggcctggagtccattagcttccagaccgctgctgaccaggctcgagagctcatcaactcctgggtggagagccagaccaacggcatgattaagaacatcctccagcccggctccgtcgatagccagaccgagatggtgctggtcaacgctatctacttcaagggcatgtgggagaaggccttcaaggacgaggatacccagaccgtgcccttccgcatgaccgagcaggaaaccaagccggtccagatgatgtaccagattggcaccttcaaggtggctgtcatgccctccgagaagatgaagattctcgagctcccctacgctagcggcgagctgtgcatgctcgtgatgctgcccgacgatgtctccggcctcgaggagctggagagctccattaccgtggagaagctgatggagtggaccagctccaacatgatggaggagcgcaagatgaaggtgttcctcccgcgcatgaagatcgaggagaagtacaacctgaccagcgtcctcatggctctgggcatgaccgacctcttcagctccagcgctaacctgtccggcatttccagcgccgagcccctcaagatgagcgaggctgtgcacgaggccttcattgagatctacgaggctggtagcgaggtggtgggtagcaccggtgctggcatggagattacctccgtcagcgaggagttccgcgccgatcaccccttcctgttcctcattaagcacaacccgaccaactccatcctgttcttcggccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 114 - DNA, 6682 bp, Artificial Sequence
  pBZ0058
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaccattgagaactacatctccgcccaggcaattgtccagggtatcagtaacccctctggtgatctgtccagcggcgctggcctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggcctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacttctgccagcagcgtgcccggcacctgtgcggccacatctgccattggtacctacagcagtgtgactgtcacctcgtggccgagtatcgtggctactggcggcaccactacgacggcttccaagtctgtcatctccaagcgcggctccattggcgctgccagcaccgagttctgcttcgacgtgttcaaggagctgaaggtgcagcacgtcaacgagaacatcttctacagccccctgagcattatctccgctctgagcatggtctacctcggtgctcgagagaacacccgagctcagattgacaaggtggtccacttcgataagatcaccggcttcggcgagcccattgagtcccagtgcggcatctccgtgagcgtccacaccagcctgaaggacatgattacccagatcaccaagccgtccgataactactccctgagcttcgctagccgcctctacgccgaggaaacctaccccattctgccggagtacctccagtgcgtgaaggagctgtacaagggcggcctcgaaaccatttccttccagaccgctgctgaccaggctcgagagctgatcaacagctgggtggagaaccagaccaacggcatgattaagaacattctccagccgggttccgtggatccccagaccgagatggtgctcgtcaacgccgtctacttcaagggcatgtgggagaaggctttcaaggacgaggatacccaggctgtgcccttccgcatgaccgagcaggagagcaagccggtccagatgatgtaccagattggctccttcaaggtggctgtcatggccagcgagaagattaagatcctggagctcccctacgcttccggcgagctgagcatgctcgtgctgctcccggacgatgtcagcggcctggagcagctcgaaaccgctatcaccctggacaagctcaccgagtggaccagctccaacgccatggaggagcgcaagatgaaggtgtacctgccccgcatgaagattgagaagaagtacaacctcacctccgtcctgatcgctctcggcatgaccgatctgttcagctccagcgctaacctcagcggcatctccagcgccgagtccctgaagatgagcgaggctattcacgaggccttcctcgagatctacgaggctggtagcgaggtggtgggtagcaccgaggctggtatggaggtgacctccgtcagcgaggagttccgcgccgatcaccccttcctgttcctcattaagcacaacccgaccaactccatcctgttcttcggccgctgcctcagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 115 - DNA, 6682 bp, Artificial Sequence
  pBZ0059
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaccattgagaactacatctccgcccaggcaattgtccagggtatcagtaacccctctggtgatctgtccagcggcgctggcctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggcctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacttctgccagcagcgtgcccggcacctgtgcggccacatctgccattggtacctacagcagtgtgactgtcacctcgtggccgagtatcgtggctactggcggcaccactacgacggcttccaagtctgtcatctccaagcgcgctagcattggcgctgccagcgccgagttctccttcgaggtgttcgacgagctgaaggctcagtaccccaaccagaacattatcttcgctccgctgtccatcctcagcgccctgtccatgctgtacctcggtgctcgcggtaacaccaaggctcagattgacaaggtggtccacttcgataagatcaccggcagcggcgaaaccgtcgagtcccagtgctccaccagcgtgaacgtccacacctccctcaaggacaccttcattcagatcaccaagcccagcgataactactccctcagcttcgcttcccgcctgtacgccgaggaaaccttccagattctcccggagtacatccagtgcgtgaaggagctgtacaaggagagcgtggaaaccgtgagcttccagaaggctgccgaccaggctcgcgagctgattaacagctgggccgagtcccagaccaacggcatgattcgcaacattctgcagccgggtagcgtggatccccagaccgagatggtgctggtcaacgctatctacttcaagggcgtgtgggctaaggccttcaaggtcgagggcacccagaccatgcccttccgcgtgaccgagcaggagagcaagccggtccagatgatgtaccagctcggctccttcaaggtgaccgacctgcccccggagaagatgcgcattctggagctcccctacgctagcggcatgctctccatgtgggtcattctgccgtacgatatcagcggcctcgagcagattgagaacgccatcaccttcgagaagctccaggagtggaccagctccaacctgatggaggagaagaccgtgaaggtctacctcccccgcatgaagatggaggagaagtacaacctgaccagcgtgctcatgggcctgggcattaccgacctcttcagctccagcgctgatctgagcggcatctccagcgtggagcgcctgaaggtgtccgaggctgtccaccaggccttcgtcgagattgacgaggctggctccgaggctgctggtattgctggtgctgctgtgaaggatgagtccgctagccccgagttcaaggtcgatcacccgttcctgttcctcattcgccacaaccccaccgatggcatcctgttcctcgcccgctgcgtgagcccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 116 - DNA, 6673 bp, Artificial Sequence
  pBZ0060
cgaaaaatcaataatcagacaacaagatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctgcaggaaggtttaaacgcatttaggtgacactatagaagtgtgtatcgctcgagggatccgaattcgaagacttggtacggagcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaccattgagaactacatctccgcccaggcaattgtccagggtatcagtaacccctctggtgatctgtccagcggcgctggcctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggcctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacttctgccagcagcgtgcccggcacctgtgcggccacatctgccattggtacctacagcagtgtgactgtcacctcgtggccgagtatcgtggctactggcggcaccactacgacggcttccaagtctgtcatctccaagcgcggcagcattggcgctgcctccatggagttctgcttcgacgtgttcaaggagctgaaggtccaccacgctaacgataacatgctctacagccccttcgctatcctgtccaccctcgccatggtgttcctgggcgctaaggacagcacccgcacccagattaacaaggtggtccacttcgacaagctcccgggcttcggcgatagcatcgaggcccagtgcggcacctccgctaacgtccacagctccctgcgcgacattctcaaccagatcaccaagcagaacgatgcttacagcttctccctggctagccgcctctacgcccaggaaacctacaccgtggtccccgagtacctgcagtgcgtgaaggagctgtaccgcggcggcctcgagtccgtcaacttccagaccgctgctgatcaggctcgaggcctgattaacgcttgggtggagagccagaccaacggcattatccgcaacatcctgcagcccagctccgtggactcccagaccgccatggtgctggtcaacgctattgccttcaagggcctctgggagaaggctttcaaggccgaggatacccagaccatccccttccgcgtgaccgagcaggagagcaagccggtccagatgatgcaccagattggctccttcaaggtggctagcatggcctccgagaagatgaagatcctggagctccccttcgctagcggcaccatgtccatgctggtgctgctcccggacgatgtcagcggcctggagcagctcgagagcaccatttccttcgagaagctcaccgagtggaccagctccagcatcatggaggagcgcaaggtgaaggtctacctgccccgcatgaagatggaggagaagtacaacctcaccagcctgctcatggccatgggcattaccgacctgttctccagctccgctaacctcagcggcattagctccgtgggctccctgaagattccccaggctgtccacgctgcttacgctgagatcaacgaggctggtcgagatgtggtgggtagcgctgaggctggtgtggatgctaccgaggagttccgcgctgatcaccccttcctcttctgcgtgaagcacattgaaaccaacgctatcctgctcttcggccgctgcgtctccccgtaggcttacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccattcctgtagtcttcttaattaagacgtcagaattctcgaggcggccgcatgtgcgtctccctatagtgagtcgtattaatttcgcgggcggaacccctatttgtttatttttctaaatacattcaaatatgtatccgctcatgagtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaatgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 117 - DNA, 47 bp, Artificial Sequence
  primer 823
tcctgagggaaagagcgttgaccgtcctctcgtatgcagaggaaatc

SEQ ID NO: 118 - DNA, 20 bp, Artificial Sequence
  primer 824
gcgcttcgacacccaagcac

SEQ ID NO: 119 - DNA, 24 bp, Artificial Sequence
  primer 825
gcgcttggagaggagactggtatc

SEQ ID NO: 120 - DNA, 22 bp, Artificial Sequence
  primer 826
gcgcttggagatgacagacttg

SEQ ID NO: 121 - DNA, 50 bp, Artificial Sequence
  primer 827
ggacggtgcttgggtgtcgaagcgcggctctattggagcggcctccatgg

SEQ ID NO: 122 - DNA, 50 bp, Artificial Sequence
  primer 828
agataccagtctcctctccaagcgcggctctattggagcggcctccatgg

SEQ ID NO: 123 - DNA, 50 bp, Artificial Sequence
  primer 829
ttccaagtctgtcatctccaagcgcggctctattggagcggcctccatgg

SEQ ID NO: 124 - DNA, 23 bp, Artificial Sequence
  primer 830
acggtcaacgctctttccctcag

SEQ ID NO: 125 - DNA, 6351 bp, Artificial Sequence
  pBZ0067
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgaagcgcggctctattggagcggcctccatggaattctgcttcgacgtgttcaaggagttgaaggtccaccacgctaacgaaaatattttctactgtccgattgctattatgtccgccctcgcgatggtatacctgggcgccaaggactctacccgaactcagatcaataaggtcgtgagattcgataagctgcctggattcggtgattcgatcgaagcgcagtgcggcacctccgtgaatgtccacagcagtctgcgagatattctgaaccaaatcactaagcccaacgatgtgtatagtttcagcctggcctctcgtctctacgcagaagagcgctaccctattctgcccgaatacttgcagtgcgtgaaggaactctaccgagggggcctggagcccataaacttccagacggctgctgaccaagcccgggagctcatcaacagttgggttgagtcacagactaacggtatcattcgcaacgtgctccagcctagcagtgttgacagccagaccgcgatggtgctagtcaacgccattgtgttcaagggtctctgggagaaggcctttaaggacgaggatacacaggctatgcccttccgcgtgaccgaacaggaaagcaagcccgtccagatgatgtaccagattggccttttccgtgtcgcaagcatggcgagcgaaaagatgaagatcctggagctcccgtttgcctcgggtaccatgtccatgctcgttctccttccggacgaagtgagcggactggagcagctggagagcatcatcaacttcgagaagttgacggagtggactagctctaacgttatggaggagcgcaagattaaggtgtatctcccccggatgaagatggaggagaagtacaacctgacctctgtgctgatggctatgggcattaccgatgtgtttagttcctccgctaacttgtccgggatctcgtcggctgagtcactcaagatttcccaagctgtccacgctgctcacgctgagatcaacgaggctggtcgtgaagtcgtcggctcggcagaagctggcgtcgatgcggcatctgtctctgaggagttccgcgccgaccacccgtttctattctgcatcaagcacatcgcaaccaatgccgttctgttcttcggcaggtgtgtgtcgccgtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 126 - DNA, 6564 bp, Artificial Sequence
  pBZ0068
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaagcgcggctctattggagcggcctccatggaattctgcttcgacgtgttcaaggagttgaaggtccaccacgctaacgaaaatattttctactgtccgattgctattatgtccgccctcgcgatggtatacctgggcgccaaggactctacccgaactcagatcaataaggtcgtgagattcgataagctgcctggattcggtgattcgatcgaagcgcagtgcggcacctccgtgaatgtccacagcagtctgcgagatattctgaaccaaatcactaagcccaacgatgtgtatagtttcagcctggcctctcgtctctacgcagaagagcgctaccctattctgcccgaatacttgcagtgcgtgaaggaactctaccgagggggcctggagcccataaacttccagacggctgctgaccaagcccgggagctcatcaacagttgggttgagtcacagactaacggtatcattcgcaacgtgctccagcctagcagtgttgacagccagaccgcgatggtgctagtcaacgccattgtgttcaagggtctctgggagaaggcctttaaggacgaggatacacaggctatgcccttccgcgtgaccgaacaggaaagcaagcccgtccagatgatgtaccagattggccttttccgtgtcgcaagcatggcgagcgaaaagatgaagatcctggagctcccgtttgcctcgggtaccatgtccatgctcgttctccttccggacgaagtgagcggactggagcagctggagagcatcatcaacttcgagaagttgacggagtggactagctctaacgttatggaggagcgcaagattaaggtgtatctcccccggatgaagatggaggagaagtacaacctgacctctgtgctgatggctatgggcattaccgatgtgtttagttcctccgctaacttgtccgggatctcgtcggctgagtcactcaagatttcccaagctgtccacgctgctcacgctgagatcaacgaggctggtcgtgaagtcgtcggctcggcagaagctggcgtcgatgcggcatctgtctctgaggagttccgcgccgaccacccgtttctattctgcatcaagcacatcgcaaccaatgccgttctgttcttcggcaggtgtgtgtcgccgtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 127 - DNA, 7965 bp, Artificial Sequence
  pBZ0069
gcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgatcattgatccaccgcccacgaggcgcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatgcgatgccctcgctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaaatgtcgttccgatctctactcgccctgagcggcctcgtctgcacagggttggcaaatgtgatttccaagcgcgcgaccttggattcatggttgagcaacgaagcgaccgtggctcgtactgccatcctgaataacatcggggcggacggtgcttgggtgtcgggcgcggactctggcattgtcgttgctagtcccagcacggataacccggactgtatgtttcgagctcagatttagtatgagtgtgtcattgattgattgatgctgactggcgtgtcgtttgttgtagacttctacacctggactcgcgactctggcctcgtcctcaagaccctcgtcgatctcttccgaaatggagataccagtctcctctccaccattgagaactacatctccgcccaggcaattgtccagggtatcagtaacccctctggtgatctgtccagcggcgctggcctcggtgaacccaagttcaatgtcgatgagactgcctacactggttcttggggacggccgcagcgagatggtccggctctgagagcaactgctatgatcggcttcgggcagtggctgcttgtatgttctccacccccttgcgtctgatctgtgacatatgtagctgactggtcaggacaatggctacaccagcaccgcaacggacattgtttggcccctcgttaggaacgacctgtcgtatgtggctcaatactggaaccagacaggatatggtgtgtttgttttattttaaatttccaaagatgcgccagcagagctaacccgcgatcgcagatctctgggaagaagtcaatggctcgtctttctttacgattgctgtgcaacaccgcgcccttgtcgaaggtagtgccttcgcgacggccgtcggctcgtcctgctcctggtgtgattctcaggcacccgaaattctctgctacctgcagtccttctggaccggcagcttcattctggccaacttcgatagcagccgttccggcaaggacgcaaacaccctcctgggaagcatccacacctttgatcctgaggccgcatgcgacgactccaccttccagccctgctccccgcgcgcgctcgccaaccacaaggaggttgtagactctttccgctcaatctataccctcaacgatggcctcagtgacagcgaggctgttgcggtgggtcggtaccctgaggacacgtactacaacggcaacccgtggttcctgtgcaccttggctgccgcagagcagttgtacgatgctctataccagtgggacaagcaggggtcgttggaggtcacagatgtgtcgctggacttcttcaaggcactgtacagcgatgctgctactggcacctactcttcgtccagttcgacttatagtagcattgtagatgccgtgaagactttcgccgatggcttcgtctctattgtggtaagtctacgctagacaagcgctcatgttgacagagggtgcgtactaacagaagtaggaaactcacgccgcaagcaacggctccatgtccgagcaatacgacaagtctgatggcgagcagctttccgctcgcgacctgacctggtcttatgctgctctgctgaccgccaacaaccgtcgtaactccgtcgtgcctgcttcttggggcgagacttctgccagcagcgtgcccggcacctgtgcggccacatctgccattggtacctacagcagtgtgactgtcacctcgtggccgagtatcgtggctactggcggcaccactacgacggcttccaagtctgtcatctccaagcgcggctctattggagcggcctccatggaattctgcttcgacgtgttcaaggagttgaaggtccaccacgctaacgaaaatattttctactgtccgattgctattatgtccgccctcgcgatggtatacctgggcgccaaggactctacccgaactcagatcaataaggtcgtgagattcgataagctgcctggattcggtgattcgatcgaagcgcagtgcggcacctccgtgaatgtccacagcagtctgcgagatattctgaaccaaatcactaagcccaacgatgtgtatagtttcagcctggcctctcgtctctacgcagaagagcgctaccctattctgcccgaatacttgcagtgcgtgaaggaactctaccgagggggcctggagcccataaacttccagacggctgctgaccaagcccgggagctcatcaacagttgggttgagtcacagactaacggtatcattcgcaacgtgctccagcctagcagtgttgacagccagaccgcgatggtgctagtcaacgccattgtgttcaagggtctctgggagaaggcctttaaggacgaggatacacaggctatgcccttccgcgtgaccgaacaggaaagcaagcccgtccagatgatgtaccagattggccttttccgtgtcgcaagcatggcgagcgaaaagatgaagatcctggagctcccgtttgcctcgggtaccatgtccatgctcgttctccttccggacgaagtgagcggactggagcagctggagagcatcatcaacttcgagaagttgacggagtggactagctctaacgttatggaggagcgcaagattaaggtgtatctcccccggatgaagatggaggagaagtacaacctgacctctgtgctgatggctatgggcattaccgatgtgtttagttcctccgctaacttgtccgggatctcgtcggctgagtcactcaagatttcccaagctgtccacgctgctcacgctgagatcaacgaggctggtcgtgaagtcgtcggctcggcagaagctggcgtcgatgcggcatctgtctctgaggagttccgcgccgaccacccgtttctattctgcatcaagcacatcgcaaccaatgccgttctgttcttcggcaggtgtgtgtcgccgtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaat

SEQ ID NO: 128 - DNA, 24 bp, Artificial Sequence
  primer 787
gatcattgatccaccgcccacgag

SEQ ID NO: 129 - DNA, 24 bp, Artificial Sequence
  primer 789
atttagtggcagatggcatcacag

SEQ ID NO: 130 - protein, 510 aa, Artificial Sequence
  Truncated GLA and synthetic linker L27
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEVNGSSFFTIAVQHRALVEGSAFATAVGSSCSWCDSQAPEILCYLQSFWTGSFILANFDSSRSAKDANTLLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDATGTYSSSSSTYSSIVDAVKTFADGFVSIVETHAASNGSMSEQYDKSDGEQLSARDLTWSYAALLTANNRRNVVPSASWGETSASSVPGTCAATSAIGTYSSVTVTSWPSIVATGGTTTTASKSVISKR